(19) United States
(12) Patent Application Publication
(10) Pub. No.: US 2021/0332024 A1
(43) Pub. Date: Oct. 28, 2021

(54) MODULATORS OF PIN1 ACTIVITY AND USES THEREOF

(71) Applicants: Yeda Research and Development Co. Ltd., Rehovot (IL); Dana-Farber Cancer Institute, Inc., Boston, MA (US); Beth Israel Deaconess Medical Center, Inc., Boston, MA (US)

(72) Inventors: Nir LONDON, Rehovot (IL); Daniel ZAIDMAN, Rehovot (IL); Christian DUBIELLA, Rehovot (IL); Nathanael S. GRAY, Jamaica Plain, MA (US); Benika Joan PINCH, Brookline, MA (US); Kun Ping LU, Newton, MA (US); Alfred Thomas LOOK, North Reading, MA (US); Shuning HE, Brookline, MA (US); Xiao Zhen ZHOU, Newton, MA (US); Xiaolan LIAN, Fuzhou (CN)

(73) Assignees: Yeda Research and Development Co. Ltd., Rehovot (IL); Dana-Farber Cancer Institute, Inc., Boston, MA (US); Beth Israel Deaconess Medical Center, Inc., Boston, MA (US)

(21) Appl. No.: 17/370,216

(22) Filed: Jul. 8, 2021

Related U.S. Application Data
(63) Continuation of application No. PCT/IL2020/050043, filed on Jan. 9, 2020.
(60) Provisional application No. 62/790,133, filed on Jan. 9, 2019.

(51) Int. Cl. C07D 333/16

(52) U.S. Cl. CPC C07D 333/16; C40B 30/04

(57) ABSTRACT

Disclosed herein are compounds comprising an electrophilic moiety and rigid moiety for use in modulating an activity of Pin1. The rigid moiety comprises at least one functional group that is capable of forming hydrogen bonds with hydrogen atoms, wherein the electrophilic moiety and the rigid moiety are arranged such that the electrophilic moiety is capable of covalently binding to the Cys113 residue of Pin1, and the rigid moiety is capable of forming hydrogen bonds with the Gln131 and His 157 residues of Pin1. Further disclosed are novel compounds having Formula Id:

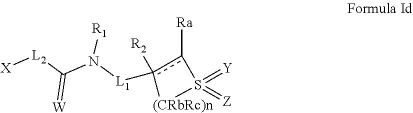

wherein the dashed line, W, X, Y, Z, Ra-Rc, R₁, R₂, L₁, L₂ and n are as defined herein, and libraries comprising such compounds. Further disclosed are methods of identifying a compound capable of modulating an activity of Pin1, by screening a library of compounds.

### RELATED APPLICATIONS

This application is a Continuation of PCT Patent Application No. PCT/IL2020/050043 having International filing date of Jan. 9, 2020, which claims the benefit of priority under 35 USC § 119(e) of U.S. Provisional Patent Application No. 62/790,133 filed on Jan. 9, 2019. The contents of the above applications are all incorporated by reference as if fully set forth herein in their entirety.

### SEQUENCE LISTING STATEMENT

The ASCII file, entitled 88213SequenceListing.txt, created on Jul. 9, 2021, comprising 2,487 bytes, submitted concurrently with the filing of this application is incorporated herein by reference.

### FIELD AND BACKGROUND OF THE INVENTION

The present invention, in some embodiments thereof, relates to pharmacology, and more particularly, but not exclusively, to newly designed compounds that covalently bind to, and/or modulate the activity of, Pin1 and to uses thereof, for example, in treating diseases associated with Pin1 activity.

Phosphorylation of Serine-Proline or Threonine-Proline motifs (pSer/Thr-Pro) by proline-directed kinases is a central signaling mechanism that is reported to be frequently deregulated in oncogenic pathways, driving cell transformation and downregulating apoptosis [Hanahan & Weinberg, Cell 2011, 144:646-674]. This motif can be isomerized (from cis to trans or trans to cis) by peptidyl-prolyl isomerase NIMA-interacting-1 (Pin1) [Lu and Zhou, Nat Rev Mol Cell Biol 2007, 8:904-916], which is the only phosphorylation-dependent isomerase amongst the approximately 30 peptidyl-prolyl cis-trans isomerases (PPIases) in the human proteome. This isomerization induces conformational changes that can impact substrate stability [Lam et al., Mol Cancer 2008, 7:91; Liao et al., Oncogene 2009, 28:2436-2445; Lee et al., Nat Cell Biol 2009, 11:97-105], activation [Chen et al., Cell Death Dis 2018, 9:883], subcellular localization [Ryo et al., Nat Cell Biol 2001, 3:793-801], and/or binding to interaction partners including Proline-directed kinases and phosphatases, which are mostly trans-specific [Xiang et al., Nature 2010, 467:729-733; Zhou et al., Mol Cell 2000, 6:873-883; Brown et al., Nat Cell Biol 1999, 1:438-443]. Pin1 is therefore an important mediator of proline-directed signaling networks, and frequently plays a role in cancer, of activating oncogenes and inactivating tumor suppressors [Chen et al., Cell Death Dis 2018, 9:883].

Several lines of evidence indicate that abnormal Pin1 activation is a key driver of oncogenesis.

Pin1 has been reported to be overexpressed and/or overactivated in at least 38 tumor types [Bao et al., Am J Pathol 2004, 164:1727-1737], by mechanisms which include transcriptional activation [Rustighi et al., Nat Cell Biol 2009, 11:133-142; Ryo et al., Mol Cell Biol 2002, 22:5281-5295] and post-translational modifications [Lee et al., Mol Cell 2011, 42:147-159; Rangasamy et al., Proc Natl Acad Sci 2012, 109:8149-8154; Chen et al., Cancer Res 2013, 73: 3951-3962; Eckerdt et al., J Biol Chem 2005, 280:36575-36583]. High expression is reported to correlate with poor clinical prognosis [Lu, Cancer Cell 2003, 4:175-180; Tan et al., Cancer Biol Ther 2010, 9:111-119], whereas polymorphisms that result in lower Pin1 expression is reported to reduce cancer risk [L₁ et al., PLoS One 2013, 8:e68148].

Pin1 has been reported to sustain proliferative signaling in cancer cells by upregulating over 50 oncogenes or growth-promoting factors [Chen et al., Cell Death Dis 2018, 9:883], including NF-κB [Ryo et al., Mol Cell 2003, 12:1413-1426], c-Myc [Farrell et al., Mol Cell Biol 2013, 33:2930-2949] and Notchl [Rustighi et al., Nat Cell Biol 2009, 11:133-142], while suppressing over 20 tumor suppressors or growth-inhibiting factors, such as FOXOs [Brenkman et al., Cancer Res 2008, 68:7597-7605], Bcl2 [Basu et al., Neoplasia 2002, 4:218-227] and RARa [Gianni et al., Cancer Res 2009, 69:1016-1026].

Furthermore, Pin1 depletion was reported to inhibit tumorigenesis in mouse models derived by mutated p53 [Girardini et al., Cancer Cell 2011, 20:79-91], activated HER2/RAS [Wulf et al., EMBO J 2004, 23:3397-3407], or constitutively expressed c-Myc [D'Artista et al., Oncotarget 2016, 7:21786-21798].

In addition, Pin1 inhibition has been reported to sensitize cancer cells to chemotherapeutics [Gianni et al., Cancer Res 2009, 69:1016-1026; Zheng et al., Oncotarget 2017, 8:29771-29784; Sajadimajd & Yazdanparast, Apoptosis 2017, 22:135-144; Ding et al., Cancer Res 2008, 68:6109-6117] and to radiation [Liu et al., Nat Cell Biol 2019, 21:203-213], and block the tumorigenesis of cancer stem cells [Rustighi et al., Nat Cell Biol 2009, 11:133-142; Ding et al., Cancer Res 2008, 68:6109-6117; Min et al., Mol Cell 2012, 46:771-783], which are involved in the development of drug resistance [Dean et al., Nat Rev Cancer 2005, 5:275-284].

Hennig et al. [Biochemistry 1998, 37:5952-5960] describes irreversible inhibition of several PPIases by juglone (5-hydroxy-1,4-naphthalenedione).

Kim et al. [Mol Cancer Ther 2009, 8:2163-2171] reports that inhibition of Pin1—e.g., by juglone—reduces angiogenesis associated with growth factor release by tamoxifen-resistant breast cancer.

Campaner et al. [Nat Commun 2017, 8:15772] reports that KPT-6566, a derivative of juglone, exhibits anti-cancer activity mediated by covalent inhibition of Pin1 and release of a quinone-mimicking drug that generates reactive oxygen species and DNA damage.

Wei et al. [Nat Med 2015, 21:457-466] reports that the anticancer activity of all-trans retinoic acid (ATRA) is mediated by inhibition of Pin1.

Kozono et al. [Nat Commun 2018, 9:3069] reports that the anti-cancer activity of the combination of arsenic trioxide and ATRA is mediated by noncovalent binding of arsenic trioxide to Pin1 and by enhancement by ATRA of arsenic trioxide cellular uptake, as well as by inhibition of Pin1 by ATRA.

However, Pin1's potential as drug target remains elusive because available Pin1 inhibitors lack the specificity and/or cell permeability to interrogate its pharmacological function in vivo [Lu & Hunter, Cell Res 2014, 24:1033-1049; Moore & Potter, Bioorganic Med Chem Lett 2013, 23:4283-4291; Fila et al., J Biol Chem 2008, 283:21714-21724].

Additional background art includes Blume-Jensen & Hunter [Nature 2001, 411:355-365]; Cheng et al. [J Med Chem 2016, 59:2005-2024]; Dahal et al. [Medchemcomm 2016, 7:864-872]; Flanagan et al. [J Med Chem 2014, 57:10072-10079]; Guo et al. [Bioorganic Med Chem Lett 2009, 19:5613-5616]; Guo et al. [Bioorganic Med Chem Lett 2014, 24:4187-4191]; Ieda et al. [Bioorganic Med Chem Lett 2018, S0960-894X(18)30990-9 (e-published)]; Leeson & Springthorpe [Nat Rev Drug Discov 2007, 6:881-890]; Lian et al. [J Hematol Oncol 2018, 11:73]; London et al. [Nat Chem Biol 2014, 10:1066-1072]; Lonsdale et al. [J Chem Inf Model 2017, 57:3124-3137]; Pawson & Scott [Trends Biochem Sci 2005, 30:283-286]; Planken et al. [J Med Chem 2017, 60:3002-3019]; Resnick et al. [J Am Chem Soc 2019, 141:8951-8968]; Ward et al. [J Med Chem 2013, 56:7025-7048]; Yang et al. [Anal Chem 2018, 90:9576-9582]; and Zhang et al. [ACS Chem Biol 2007, 2:320-328].

### SUMMARY OF THE INVENTION

According to an aspect of some embodiments of the invention, there is provided a compound for use in modulating an activity of Pin1, the compound comprising an electrophilic moiety and rigid moiety that comprises at least one functional group that is capable of forming hydrogen bonds with hydrogen atoms, wherein the electrophilic moiety and the rigid moiety are arranged such that the electrophilic moiety is capable of covalently binding to the Cys113 residue of Pin1, and the rigid moiety is capable of forming hydrogen bonds with the Gln131 and His 157 residues of Pin1.

According to an aspect of some embodiments of the invention, there is provided compound having Formula Id:

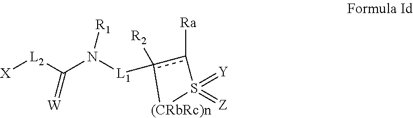

wherein:

the dashed line represents a saturated or non-saturated bond;

W is selected from the group consisting of O, S and NR₃;

X is halo;

Y and Z are each independently selected from the group consisting of O, S and NH;

Ra-Rc are each hydrogen;

L₁ is a bond or alkylene;

L₂ is alkylene;

n is 1, 2, 3 or 4;

R₁ is selected from the group consisting of —CH₂—C(CH₃)₃, —CH₂—CH(CH₃)₂, a triazole, and alkyl substituted by a triazole and/or by a 5- or 6-membered cycloalkyl;

R₂ is selected from the group consisting of hydrogen and alkyl when the dashed line represents a saturated bond, and R₂ is absent when the dashed line represents an unsaturated bond; and

R₃ is selected from the group consisting of hydrogen, alkyl, alkenyl, alkynyl, cycloalkyl, heteroalicyclic, aryl and heteroaryl.

According to an aspect of some embodiments of the invention, there is provided a screening library comprising at least 30 compounds having Formula Id.

According to an aspect of some embodiments of the invention, there is provided a method of modulating an activity of Pin1, the method comprising contacting the Pin1 with a compound according to any of the respective embodiments described herein.

According to an aspect of some embodiments of the invention, there is provided a method of identifying a compound capable of modulating an activity of Pin1, the method comprising screening a library comprising at least 30 compounds having Formula IV:

E′-L′₁-V Formula IV

wherein:

E′ is an electrophilic moiety, capable of forming a covalent bond when reacted with a thiol;

L′₁ is a linking moiety;

V is a moiety featuring at least two functional groups that are capable of forming hydrogen bonds, and optionally further features at least one lipophilic group,

for compounds that are capable of interacting with a Cys113 residue of Pin1 via the electrophilic moiety, of interacting at least with the Gln131 and His 157 residues of Pin1 via the functional groups, and optionally of interacting with at least one amino acid residue in a hydrophobic patch of Pin1 via the at least one lipophilic group,

wherein a compound identified as capable of interacting at least with the Cys113 residue and the Gln131 and His 157 residues of Pin1 is identified as capable of modifying an activity of Pin1.

According to an aspect of some embodiments of the invention, there is provided a method of identifying a compound capable of modulating an activity of Pin1, the method comprising:

a) contacting a library comprising at least 30 compounds represented by Formula Ic:

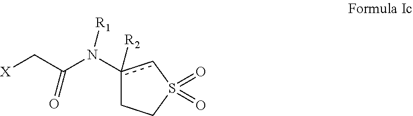

wherein:

the dashed line represents a saturated or non-saturated bond;

X is halo;

R₁ is selected from the group consisting of alkyl, alkenyl, alkynyl, cycloalkyl, heteroalicyclic, aryl and heteroaryl; and

R₂ is selected from the group consisting of hydrogen and alkyl when the dashed line represents a saturated bond, and R₂ is absent when the dashed line represents an unsaturated bond,

with Pin1 under conditions that allow nucleophilic substitution of X by a Cys113 residue of Pin1; and

b) determining which compounds covalently bound Pin1, wherein a compound which covalently binds to Pin1 is identified as being capable of modulating an activity of Pin1.

According to an aspect of some embodiments of the invention, there is provided a screening library comprising at least 30 compounds represented by Formula Ic:

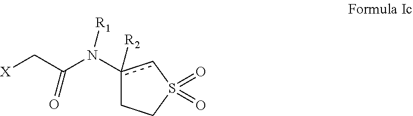

wherein:

the dashed line represents a saturated or non-saturated bond;

X is halo;

R₁ is selected from the group consisting of alkyl, alkenyl, alkynyl, cycloalkyl, heteroalicyclic, aryl and heteroaryl; and

R₂ is selected from the group consisting of hydrogen and alkyl when the dashed line represents a saturated bond, and R₂ is absent when the dashed line represents an unsaturated bond.

According to some of any of the embodiments described herein, the electrophilic moiety comprises a haloalkyl.

According to some of any of the embodiments described herein, the electrophilic moiety comprises a haloacetamide.

According to some of any of the embodiments described herein, the functional group is capable of forming a hydrogen bond with a backbone amide hydrogen of the Gln131 and/or with an imidazole NH of the His157.

According to some of any of the embodiments described herein, the hydrogen bond links an atom of the functional group to a nitrogen atom of the Gln131 or His157, such that a distance between the atom of the functional group and the nitrogen atom of the Gln131 or His157 is in a range of from 2.5 to 3.5 Å.

According to some of any of the embodiments described herein, the functional group is an oxygen atom.

According to some of any of the embodiments described herein, the rigid moiety comprises a sulfone group.

According to some of any of the embodiments described herein, the rigid moiety is or comprises a sulfolane or a sulfolene.

According to some of any of the embodiments described herein, the compound further comprising a hydrophobic moiety.

According to some of any of the embodiments described herein relating to a hydrophobic moiety, the hydrophobic moiety forms a hydrophobic interaction with Ser115, Leu122 and/or Met130 of Pin1.

According to some of any of the embodiments described herein, the compound has a molecular weight lower than 500 Da.

According to some of any of the embodiments described herein, the compound is represented by Formula I:

E-L₁-G(F)m Formula I

wherein:

E is an electrophilic moiety (according to any of the respective embodiments described herein);

L₁ is a bond or a linking moiety according to any of the respective embodiments described herein);

G is a rigid moiety according to any of the respective embodiments described herein);

F are each a functional moiety forming hydrogen bonds (according to any of the respective embodiments described herein); and

m is 2, 3 or 4.

According to some of any of the embodiments described herein, the compound is represented by Formula Ia:

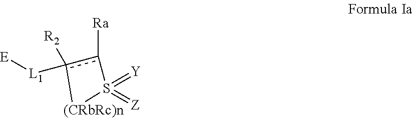

wherein:

the dashed line represents a saturated or non-saturated bond;

Y and Z are each independently selected from the group consisting of O, S and NH;

R₂ and Ra-Rc are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond; and

n is 1, 2, 3 or 4.

According to some of any of the embodiments described herein, the compound is represented by Formula Ib:

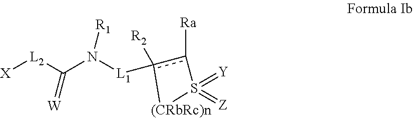

wherein:

W is selected from the group consisting of O, S and NR₃;

X is halo;

Ra-Rc are each hydrogen;

L₁ is a bond or alkylene;

L₂ is alkylene; and

R₁ and R₃ are each independently selected from the group consisting of hydrogen, alkyl, alkenyl, alkynyl, cycloalkyl, heteroalicyclic, aryl and heteroaryl.

According to some of any of the respective embodiments described herein, L₂ is methylene.

According to some of any of the respective embodiments described herein, W is O.

According to some of any of the respective embodiments described herein, n is 2.

According to some of any of the respective embodiments described herein, Y and Z are each O.

According to some of any of the respective embodiments described herein, L₁ is a bond.

According to some of any of the embodiments described herein, the compound is represented by Formula Ic:

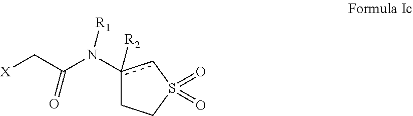

wherein:

the dashed line represents a saturated or non-saturated bond;

X is halo;

R₁ is selected from the group consisting of alkyl, alkenyl, alkynyl, cycloalkyl, heteroalicyclic, aryl and heteroaryl; and

R₂ is selected from the group consisting of hydrogen and alkyl when the dashed line represents a saturated bond, and R₂ is absent when the dashed line represents an unsaturated bond.

According to some of any of the respective embodiments described herein, X is chloro.

According to some of any of the respective embodiments described herein, R₁ has Formula II:

—CH₂—R′₁ Formula II

wherein R′₁ is selected from the group consisting of alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

According to some of any of the embodiments described herein relating to Formula II, R′₁ is a tertiary alkyl, alkenyl, alkynyl, cycloalkyl or heteroalicyclic.

According to some of any of the embodiments described herein relating to Formula II, R′₁ is a substituted or unsubstituted t-butyl.

According to some of any of the respective embodiments described herein, R₁ or R′₁ is heteroaryl.

According to some of any of the embodiments described herein relating to an R₁ or R′₁ which is heteroaryl, the heteroaryl is a triazole.

According to some of any of the embodiments described herein relating to a triazole, the triazole has Formula III:

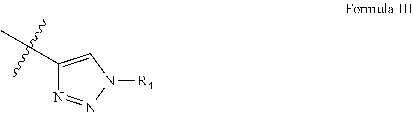

wherein R₄ is selected from the group consisting of alkyl, alkenyl, alkynyl, cycloalkyl, heteroalicyclic, aryl and heteroaryl.

According to some of any of the embodiments described herein relating to Formula III, R₄ is a substituted or unsubstituted phenyl.

According to some of any of the embodiments described herein relating to Formula III, R₄ is a phenyl substituted by a substituent selected from the group selected from hydroxy, hydroxyalkyl, halo, alkoxy, carbonyl, carboxy and sulfonamido.

According to some of any of the embodiments described herein relating to Formula III, R₄ is p-methoxycarbonylphenyl.

According to some of any of the respective embodiments described herein, the dashed line represents a saturated bond.

According to some of any of the respective embodiments described herein, R₂ is hydrogen.

According to some of any of the embodiments described herein, the compound is for use in treating a condition in which modulating an activity of Pin1 is beneficial.

According to some of any of the embodiments described herein relating to a condition in which modulating an activity of Pin1 is beneficial, the condition is a proliferative disease or disorder and/or an immune disease or disorder.

According to some of any of the embodiments described herein relating to a proliferative disease or disorder, the proliferative disease or disorder is a cancer.

According to some of any of the embodiments described herein relating to a proliferative disease or disorder, the proliferative disease or disorder is selected from the group consisting of a pancreatic cancer, a neuroblastoma, a prostate cancer, an ovarian carcinoma, and a breast adenocarcinoma.

According to some of any of the embodiments described herein relating to a proliferative disease or disorder, the proliferative disease or disorder is a pancreatic cancer.

According to some of any of the embodiments described herein relating to a proliferative disease or disorder, the proliferative disease or disorder is a neuroblastoma.

According to some of any of the embodiments described herein relating to screening a library, the screening is by computational docking.

According to some of any of the embodiments described herein relating to screening a library, the method further comprises contacting the identified compound with Pin1, to thereby determine if the compound binds to Pin1 and/or modulate an activity of Pin1, wherein a compound that is determined as capable of binding to Pin1 and/or modulating an activity of Pin1, is identified as capable of modifying an activity of Pin1.

According to some of any of the embodiments described herein relating to screening a library, the method further comprises screening the library for low reactivity with a thiol other than Cys113 of Pin1.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents an exemplary compound determined to covalently bind to Pin1, using an electrophilic library screen and intact protein mass spectroscopic (MS) labeling (200 μM compound for 24 hours at 4° C.).

FIG. 2 presents a pie chart showing analysis of the Pin1 screening hits: 48 hits labeled Pin1 (>75%) out of 993 fragments, and 9 of these 48 top hits (18.75%) are chloroacetamides that share cyclic sulfone scaffolds as common motif (right).

FIG. 3 depicts the structures of 9 compounds which share a similar structural motif (containing a sulfolane or sulfolene moiety), from among the 48 top hits from an electrophilic library screen.

FIG. 4 presents predicted binding modes for exemplary compounds bound to Pin1, as determined by docking simulations: A) the phenyl and cyclohexyl groups of PCM-0102755 (purple) and PCM-0102760 (cyan), respectively, protrude into a hydrophobic cavity build up by Met130, Gln131 and Phe134; and B) the cyclopropyl group of PCM-0102832 (orange) covers a shallow hydrophobic patch formed by Ser115, Leu122 and Met130, whereas the ethyl group of PCM-0102105 (brown) and the cyclopentyl moiety of PCM-0102313 (light brown), respectively, protrude into the solvent.

FIG. 5 depicts the structures of an exemplary set of tested compounds designed based on preliminary results (“second generation”).

FIG. 6 depicts the structures of the top 10 binders of Pin1 from the exemplary set depicted in FIG. 5, as well as those of a non-reactive (chlorine-free) control compound (Pin1-3-AcA) and juglone (a known Pin1 inhibitor).

FIG. 7 depicts compounds with no Pin1 labeling at 2 μM for 1 hour (upper row) and analogous compounds (lower row) with an additional methylene (between amide and lipophilic group) which exhibited 27-65% labeling of Pin1 under the same conditions.

FIG. 8 depicts the structures of an exemplary set of tested compounds designed based on previous results (“third generation”).

FIG. 9 presents a graph showing percentage of Pin1-labeling as a function of reactivity (quantified as log(k)) for the top ten hits from an exemplary set of tested compounds (“second generation”), and the lack of correlation (R²=0.0029) between labeling percentage and reactivity.

FIG. 10 presents a bar graph showing the reactivity towards thiols of the top ten hits from an exemplary set of tested compounds (“second generation”), using a DTNB (dithionitrobenzoic acid) assay.

FIG. 11 presents a bar graph showing the reactivity towards thiols of the top ten hits from an exemplary set of tested compounds (“third generation”), using a DTNB (dithionitrobenzoic acid) assay.

FIG. 12 presents a graph showing catalytic activity of Pin1(%) as a function of concentration of an exemplary compound (Pin1-3) or juglone as positive control.

FIG. 13 presents a graph showing binding of exemplary compounds to Pin1, as determined by fluorescence polarization of an N-terminal fluorescein-labeled peptide (Bth-D-phosThr-Pip-Nal), as a function of compound concentration upon incubation for 14 hours at room temperature (juglone served as positive control and non-reactive Pin1-3-AcA served as negative control).

FIGS. 14A and 14B present graphs showing percentage of bound Pin1-3 as a function of time (FIG. 14A) and a plot of rate as a function of Pin1-3 concentration for determining K_{inact} and Kᵢ (FIG. 14B).

FIG. 15 presents a graph showing percentage of Pin1-labeling as a function of reactivity (quantified as log(k)) for the top ten hits from an exemplary set of tested compounds (“second generation”); the reactivities of Pin1-3, Pin1-3-13 and cytotoxic fragments (Tox) are delineated by dashed lines.

FIG. 16 presents a graph showing percentage of Pin1-labeling as a function of reactivity (quantified as log(k)) for the top ten hits from an exemplary set of tested compounds (“third generation”).

FIG. 17 presents an X-ray crystal structure showing continuous electron density between Cys113 and Pin1-3.

FIG. 18 presents an X-ray crystal structure of Pin1 in complex with Pin1-3 (1.4 Å resolution); hydrogen-bonds are depicted as dashed lines.

FIG. 19 presents a superposition of the X-ray crystal structure shown in FIG. 18 (Pin1 in white, Pin1-3 in salmon) with an X-ray crystal structure (pdb code: 6DUN; 1.6 Å resolution) of Pin1 (cyan) in complex with arsenic trioxide (purple); the sulfolane moiety of Pin1-3 and arsenic trioxide occupy the hydrophobic Pro-binding pocket formed by M130, Q131, F134, Thr152 and H157, and the sulfonyl oxygens (red) of Pin1-3 and arsenic trioxide similarly mediate hydrogen bonds with the backbone amide of Q131 and the imidazole NH of H157.

FIG. 20 presents the structure of the exemplary desthiobiotin probe Pin1-3-DTB.

FIG. 21 presents a graph showing fluorescence polarization (expressed as a normalized mP value) as a function of concentration of Pin1-3, Pin1-3-DTB and Pin1-3-AcA.

FIG. 22 presents a Western blot showing binding of 0.1, 0.25, 0.5 or 1 μM Pin1-3-DTB to Pin1 upon incubation for 1 hour in PAT8988T cell lysates.

FIG. 23 presents a Western blot showing binding of 1 μM Pin1-3-DTB to Pin1 following exposure of PATU-8988T cells to 1 μM Pin1-3 for 0, 0.5, 1, 2 or 4 hours; Pin1-3 competes with the probe Pin1-3-DTB for Pin1 binding in a time-dependent manner (cells were incubated with Pin1-3 for the indicated times, followed by lysis and incubation for with Pin1-3-DTB).

FIG. 24 presents a Western blot showing binding of 1 μM Pin1-3-DTB to Pin1 following exposure of PATU-8988T cells to 0.25, 0.5 or 1 μM Pin1-3 or 1 μM Pin1-3-AcA; Pin1-3 competes with the probe Pin1-3-DTB for Pin1 binding in cells in a dose-dependent manner, with full engagement of Pin1 at 1 μM, whereas the non-reactive analog Pin1-3-AcA does not (cells were incubated with the tested compound at the indicated concentration for 5 hours, followed by lysis and incubation for 1 hour with Pin1-3-DTB).

FIG. 25 presents a Western blot showing binding of 1 μM Pin1-3-DTB to Pin1 following exposure of PATU-8988T cells to 1 μM Pin1-3 for 24, 48 or 72 hours; significant engagement (>50%) of Pin1 by Pin1-3 is still observed after 72 hours (cells were incubated with or without Pin1-3 for the indicated times, followed by lysis and incubation with Pin1-3-DTB).

FIG. 26 presents a Western blot showing binding of Pin1-3-DTB to Pin1 following exposure of IMR32 cells to 0.25, 0.5 or 1 μM Pin1-3 or 1 μM Pin1-3-AcA; Pin1-3 competes with the probe Pin1-3-DTB for Pin1 binding in cells in a dose-dependent manner, with full engagement of Pin1 at 1 μM, whereas the non-reactive analog Pin1-3-AcA does not.

FIG. 27 presents a Western blot showing binding of 1 μM Pin1-3-DTB to Pin1 with or without administration of 10 or 20 mg/kg Pin1-3 to mice; significant engagement of Pin1 by Pin1-3 is observed for at least some of the samples at each Pin1-3 dosage (mice were treated with the indicated amounts of Pin1 by oral gavage, once per day for three days, and then the spleens were lysed and incubated with Pin1-3-DTB).

FIG. 28 presents a schematic depiction of an exemplary CITe-Id experiment for identifying competitively labeled cysteine throughout the proteome following a dose response treatment with Pin1-3.

FIG. 29 presents a graph showing results of an exemplary CITe-Id experiment (performed as depicted in FIG. 28); of 162 identified labeled cysteine residues, only C113 in Pin1 (indicated by arrow) is labeled in a dose-dependent manner.

FIG. 30 presents a bar graph showing the dose-dependence of Pin1 C113 labeling by Pin1-3, as determined by an exemplary CITe-Id experiment (performed as depicted in FIG. 28).

FIG. 31 presents a schematic depiction of an exemplary rdTOP-ABPP experiment for assessing Pin1-3 proteomic selectivity.

FIG. 32 presents a graph showing the competition ratio of the top 25 peptides identified in the rdTOP-ABPP experiment (as depicted in FIG. 31).

FIG. 33 presents a graph showing normalized cell growth of wild-type 8988T pancreatic cancer cells as a function of time upon incubation with 1 μM of Pin1-3 or vehicle (DMSO) (*** p<0.001, **** p<0.0001).

FIG. 34 presents a graph showing normalized cell growth of Pin1-knockout 8988T pancreatic cancer cells as a function of time upon incubation with 1 μM of Pin1-3 or vehicle (DMSO).

FIG. 35 presents Western blot images showing Pin1 expression in wild-type (813) and Pin1-knockout (826) 8988T pancreatic cancer cells (tubulin expression used as loading control).

FIG. 36 presents a graph showing normalized cell growth of PC3 cancer cells as a function of time upon incubation with 1 or 2.5 μM Pin1-3, or 2.5 μM Pin1-3-AcA or vehicle (DMSO).

FIG. 37 presents a graph showing normalized cell growth of Kuramochi cancer cells as a function of time upon incubation with 1 or 2.5 μM Pin1-3, or 2.5 μM Pin1-3-AcA or vehicle (DMSO) (**** p<0.0001).

FIG. 38 presents a graph showing normalized cell growth of MDA-MB-468 cancer cells as a function of time upon incubation with 1 or 2.5 μM Pin1-3, or 2.5 μM Pin1-3-AcA or vehicle (DMSO) (**** p<0.01).

FIG. 39 presents a bar graph showing organoid growth (as determined by luminescence measurement) in wild-type (WT) and Pin1-knockout (KO) 8988T pancreatic cancer cells following treatment with 1 μM Pin1-3 or Pin1-3-AcA, or vehicle (DMSO) (**** p<0.0001).

FIG. 40 presents a comparison of changes in RNA levels in Mino B cells treated with either 1 μM Pin1-3 or DMSO (6 hours, in triplicates), in which each dot represents the p-value for significance of that change (Student's t-test) as a function of the Log₂ fold change of a transcript; 206 genes were downregulated in a significant manner (p=0.05 indicated by dotted line).

FIG. 41 presents a bar graph showing results of a gene set enrichment analysis using Enrichr against the ENCODE TF ChIP-seq set; two of the most enriched sets are Myc target genes from different cell lines.

FIG. 42 presents representative images of embryos (7 dpf) of Tg(dβh:EGFP) and Tg(dβh:MYCN;dβh:EGFP) transgenic zebrafish (upper two images) and Tg(dβh:MYCN;dβh:EGFP) transgenic zebrafish following a 4 day treatment (from 3 to 7 dpf) with 50 or 100 μM of Pin1-3 (lower two images), in which primordial superior cervical ganglia (SCG) and intrarenal gland (IRG) (observed via EGFP fluorescence) are highlighted by dotted circles.

FIG. 43 presents the distribution of the normalized neuroblastoma tumor area in the primordial superior cervical ganglia (SCG) and intrarenal gland (IRG) zebrafish embryos (7 dpf) following a 4 day treatment (from 3 to 7 dpf) with 0, 25, 50 or 100 μM of Pin1-3MYCN hyperproliferative effect on neuroblasts shown by comparison between EGFP fluorescence of dβh:EGFP control reporter line with ˜10-fold cross-sectional area in untreated (0 μM) MYCN transgenic line (dβh:MYCN/EGFP) (p values determined by Mann-Whitney test with confidence intervals of 95% for determining significance; quantitative data shown as median).

FIG. 44 presents representative images of zebrafish embryos transplanted with neuroblastoma cells isolated from a 4-month old Tg(dβh:MYCN;dβh:EGFP) donor zebrafish and treated with DMSO control (CTR) or 100 μM Pin1-3 added to the fish water.

FIG. 45 presents the distribution of the normalized EGFP-positive tumor area in zebrafish embryos treated with DMSO or 100 μM Pin1-3 added to the fish water (p values determined by Mann-Whitney test with confidence intervals of 95% for determining significance; quantitative data shown as median).

FIGS. 46A and 46B presents representative flow cytometric plots (FIG. 46A) and a graph (FIG. 46B) showing quantification of FAS^{Hi} CD38⁻ germinal center (GC) cells in WT mice treated with vehicle or Pin1-3, 11 days after immunization with NP-OVA (** indicates p<0.01 in two tailed Student's t-test).

FIG. 47 presents representative images of PDAC cells upon being treated with Pin1-3 for 3 days (scale bars=100 μm).

FIG. 48 presents graphs showing PDAC cell growth as a function of Pin1-3 concentration following treatment with Pin1-3 for 3 days.

FIG. 49 presents a Western blot images showing Pin1 levels in PDAC cells treated with Pin1-3 for 3 days.

FIG. 50 presents representative images of PDAC organoids upon being treated with Pin1-3 for 7 days (scale bars=100 μm).

FIG. 51 presents graphs showing PDAC organoid area as a function of Pin1-3 concentration following treatment with Pin1-3 for 7 days.

FIG. 52 presents representative images of PDX tumors in an orthotopic xenograft mouse model with or without administration of 2 or 4 mg/kg Pin1-3.

FIG. 53 presents a graph showing PDX tumor volume in an orthotopic xenograft mouse model with or without administration of 2 or 4 mg/kg Pin1-3.

FIG. 54 presents a graph showing PDX tumor volume as a function of time, in an orthotopic xenograft mouse model with or without administration of 2 or 4 mg/kg Pin1-3.

FIG. 55 presents representative images of KPC mouse derived tumor in an orthotopic xenograft mouse model with or without administration of 40 mg/kg Pin1-3.

FIG. 56 presents a graph showing KPC tumor volume in an orthotopic xenograft mouse model with or without administration of 40 mg/kg Pin1-3.

FIG. 57 presents a graph showing survival in a KPC orthotopic xenograft mouse model with or without administration of 20 or 40 mg/kg Pin1-3.

### DESCRIPTION OF SPECIFIC EMBODIMENTS OF THE INVENTION

The present invention, in some embodiments thereof, relates to pharmacology, and more particularly, but not exclusively, to newly designed compounds that covalently bind to, and/or modulate the activity of, Pin1 and to uses thereof in, for example, treating diseases associated with Pin1 activity.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

The present inventors have uncovered new compounds for effectively and selectively modulating the activity of Pin1, by laboriously screening compounds capable of covalently reacting with the protein, and studying the relationship between structure and activity and off-target toxicity.

While reducing the present invention to practice, the inventors have uncovered exemplary compounds which selectively and covalently react with the active site (catalytic domain) of Pin1, as well as the effects of selective modulation of Pin1 activity in various physiological models.

As used herein, the phrase “catalytic domain” describes a region of an enzyme, Pin1, in which the catalytic reaction occurs. This phrase therefore describes this part of an enzyme in which the substrate and/or other components that participate in the catalytic reaction interacts with the enzyme. In the context of the present embodiments, this phrase is particularly used to describe this part of an enzyme (a Pin1) to which the substrate binds during the catalytic activity (e.g., phosphorylation). This phrase is therefore also referred to herein and in the art, interchangeably, as “substrate binding pocket”, “catalytic site” “active site” and the like.

As used herein, the phrases “binding site”, “catalytic binding site” or “binding subsite”, which are used herein interchangeably, describe a specific site in the catalytic domain that includes one or more reactive groups through which the interactions of the enzyme with the substrate and/or an inhibitor can be effected. Typically, the binding site is composed of one or two amino acid residues, whereby the interactions typically involve reactive groups at the side chains of these amino acids.

As is well known in the art, when an enzyme interacts with a substrate or an inhibitor, the initial interaction rapidly induces conformational changes, in the enzyme and/or substrate and/or inhibitor, that strengthen binding and bring enzyme's binding sites close to functional groups in the substrate or inhibitor. Enzyme-substrate/inhibitor interactions orient reactive groups present in both the enzyme and the substrate/inhibitor and bring them into proximity with one another. The binding of the substrate/inhibitor to the enzyme aligns the reactive groups so that the relevant molecular orbitals overlap.

Thus, an inhibitor of an enzyme is typically associated with the catalytic domain of the enzyme such that the reactive groups of the inhibitor are positioned in sufficient proximity to corresponding reactive groups (typically side chains of amino acid residues) in the enzyme catalytic binding site, so as to allow the presence of an effective concentration of the inhibitor in the catalytic binding site and, in addition, the reactive groups of the inhibitor are positioned in a proper orientation, to allow overlap and thus a strong chemical interaction and low dissociation. An inhibitor therefore typically includes structural elements that are known to be involved in the interactions, and may also have a restriction of its conformational flexibility, so as to avoid conformational changes that would affect or weaken its association with catalytic binding site.

The present inventors have uncovered that a series of structurally similar small molecules efficiently bind, covalently, to the Cys113 residue of Pin1, and have designed, based on these findings, and successfully practiced, novel small molecules that are capable of interacting with Pin1. The present inventors have identified that the structural features of the newly designed compounds that allow efficient interaction within the catalytic domain of Pin1, for example, such that reactivity with Cys113 is far higher than with other thiol groups.

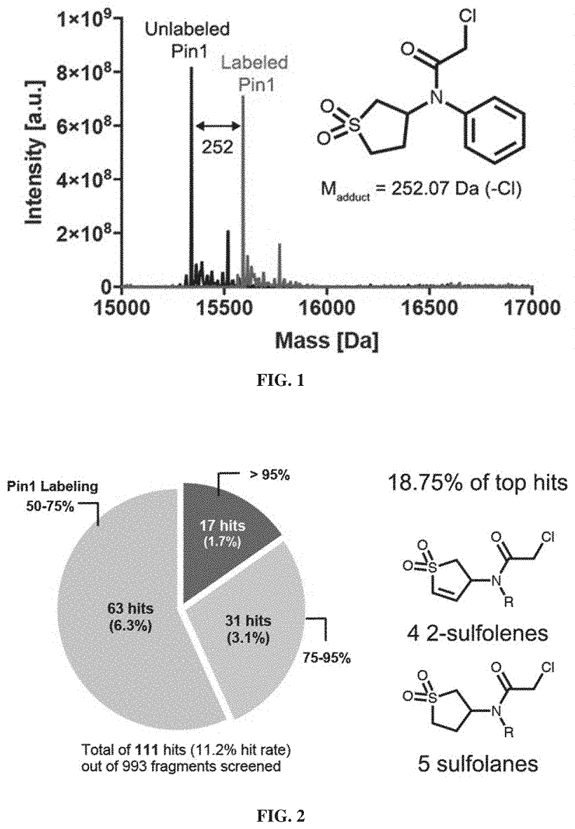

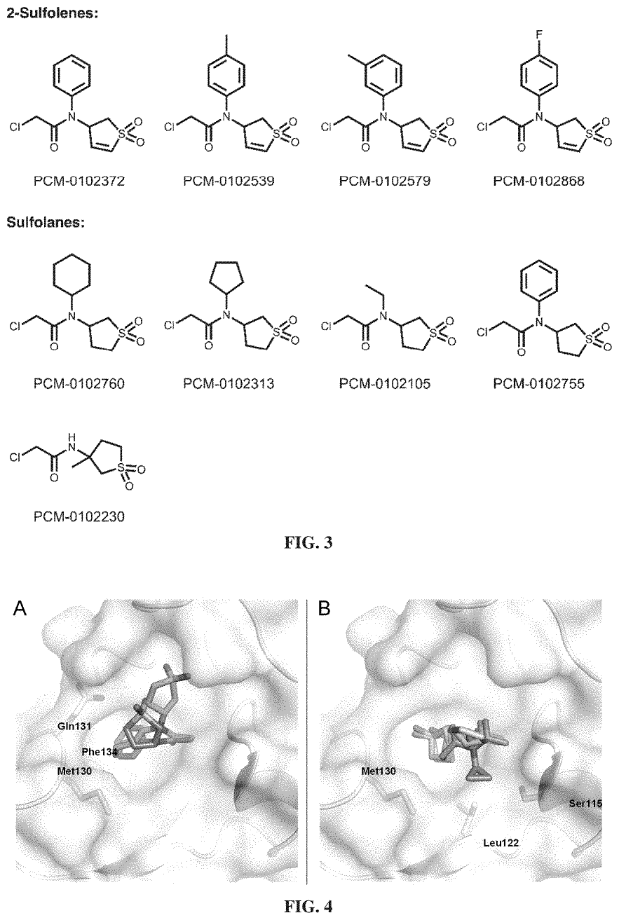

Referring now to the drawings, FIG. 1 illustrates the use of intact protein mass spectroscopic labeling to screen an electrophilic library for compounds which covalently bind to Pin1. FIG. 2 briefly summarizes the results of the electrophilic library screen, showing a correlation between activity and a structure comprising a cyclic sulfone moiety. FIG. 3 presents all of the top hits which comprise a cyclic sulfone moiety.

FIG. 4 shows predicted binding modes for compounds with a cyclic sulfone moiety.

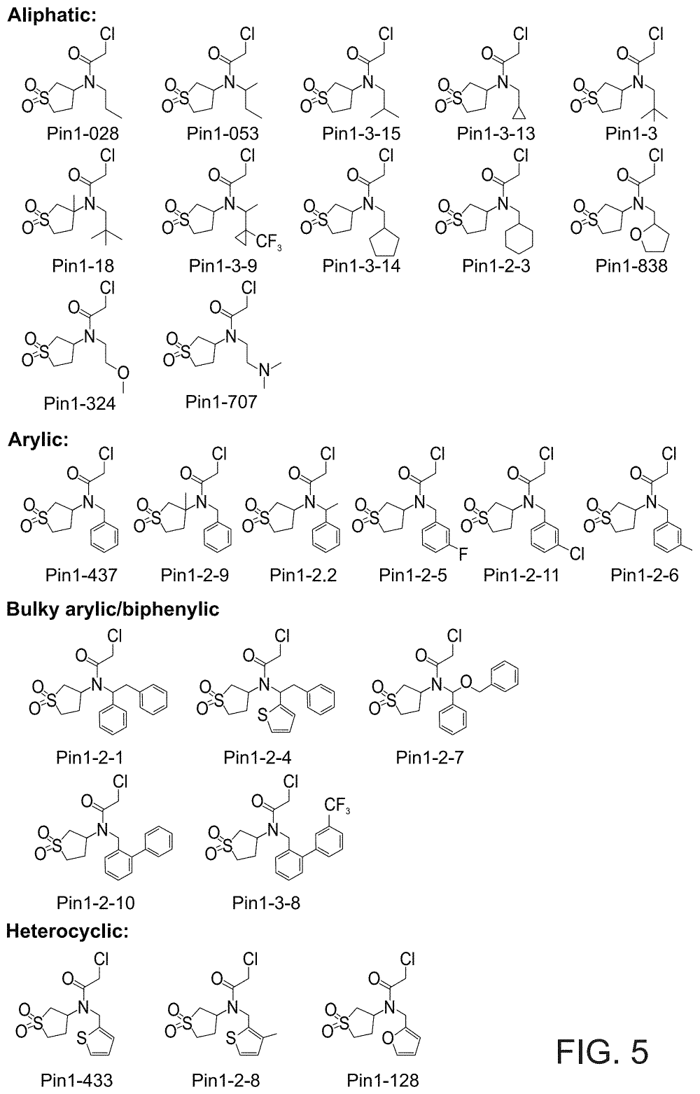

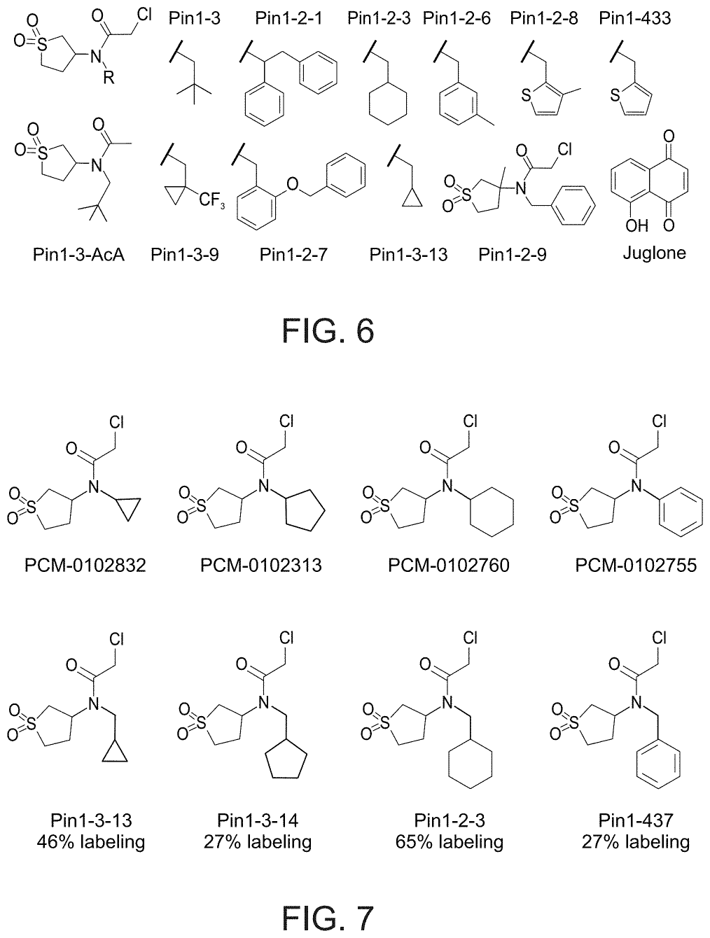

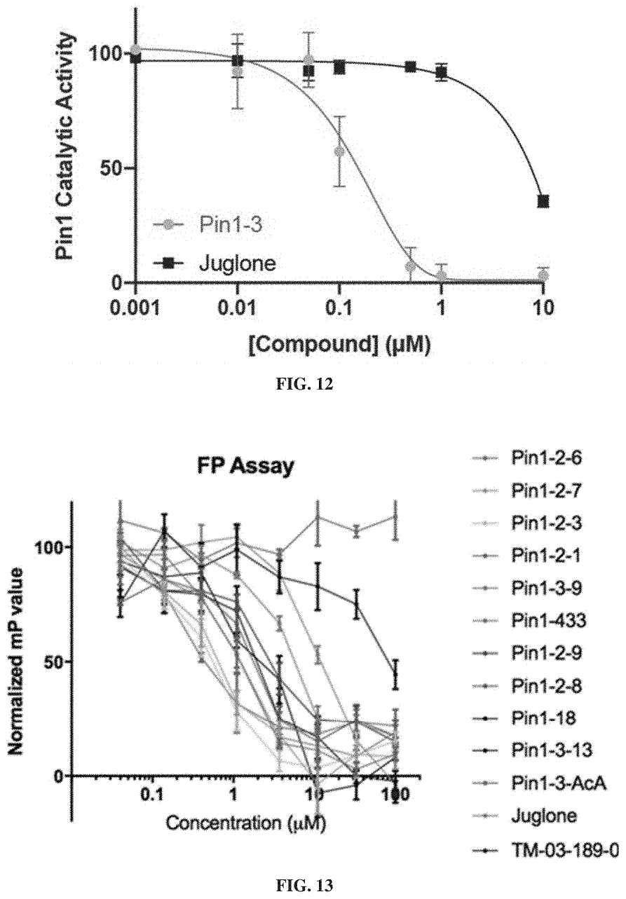

FIGS. 5-6 show second generation compounds for assessing the effect of amide substituents of N-(sulfolan-3-yl)-2-chloroacetamides on Pin1-labeling activity. Similarly, FIG. 8 shows additional (third generation) compounds, generated by click chemistry, for assessing the effect of amide substituents of N-(sulfolan-3-yl)-2-chloroacetamides on Pin1-labeling activity. FIGS. 12-14B show that Pin1-labeling by exemplary compounds is associated with inhibition of enzymatic activity. FIG. 7 shows that a methylene linker adjacent to the amide nitrogen atom is associated with enhanced activity.

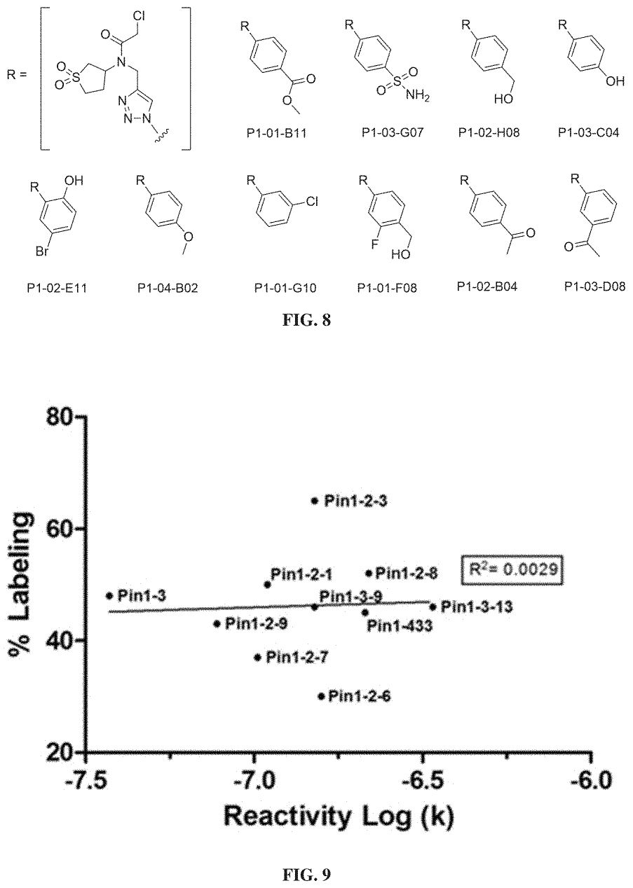

FIGS. 9-11 and 15-16 shows that some compounds, such as Pin1-3 and P1-01-B11, exhibit a particularly low amount of non-specific reactivity towards thiols and cytotoxicity, for a given degree of Pin1-labeling.

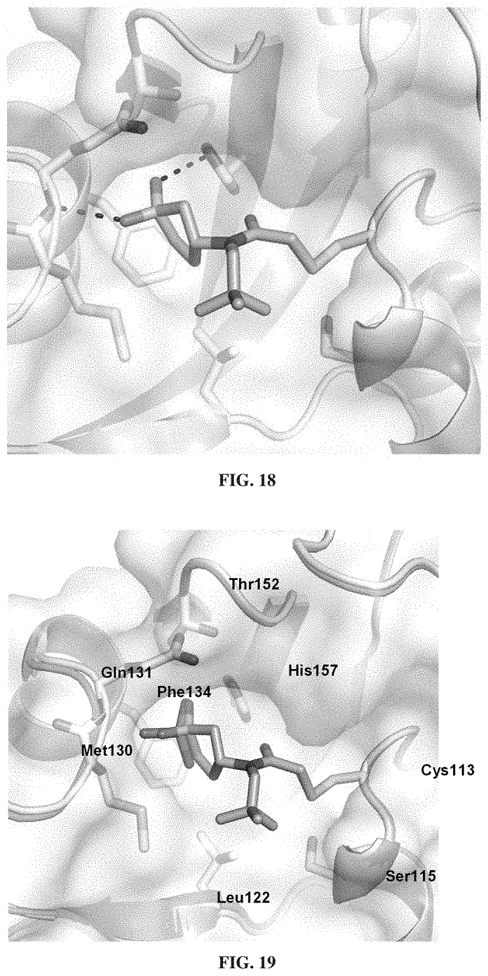

FIGS. 18 and 19 show the structure of an exemplary compound covalently bound to Cys113 of Pin1, and further bound by hydrogen bonds between the sulfone oxygens and Gln131 and His157, as determined by X-ray crystallography.

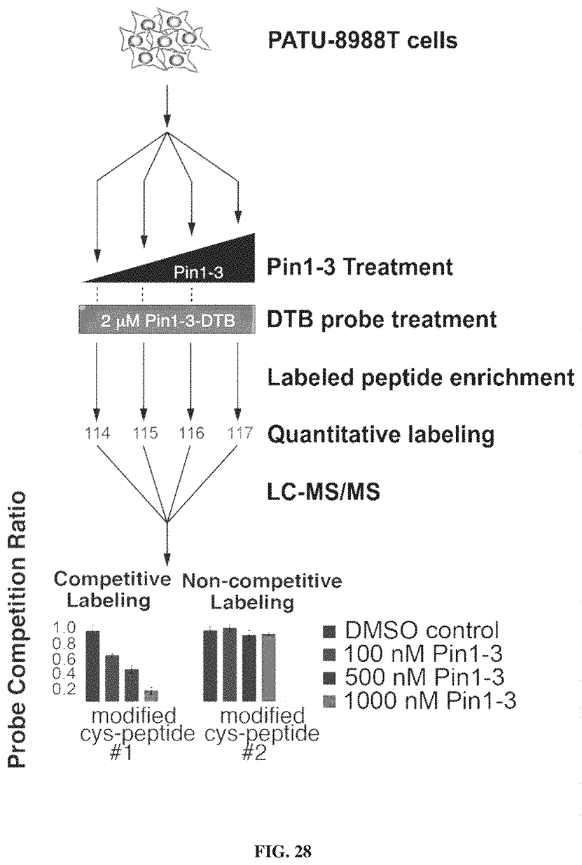

FIGS. 21-27 show that exemplary compounds engage Pin1 in a time-dependent and dose-dependent manner in vitro and in vivo, and that the covalently reactive chloroacetamide group is important for Pin1-labeling, as a corresponding acetamide does not effectively bind to Pin1. FIGS. 28-32 show selectivity towards Pin1, as compared with other peptides.

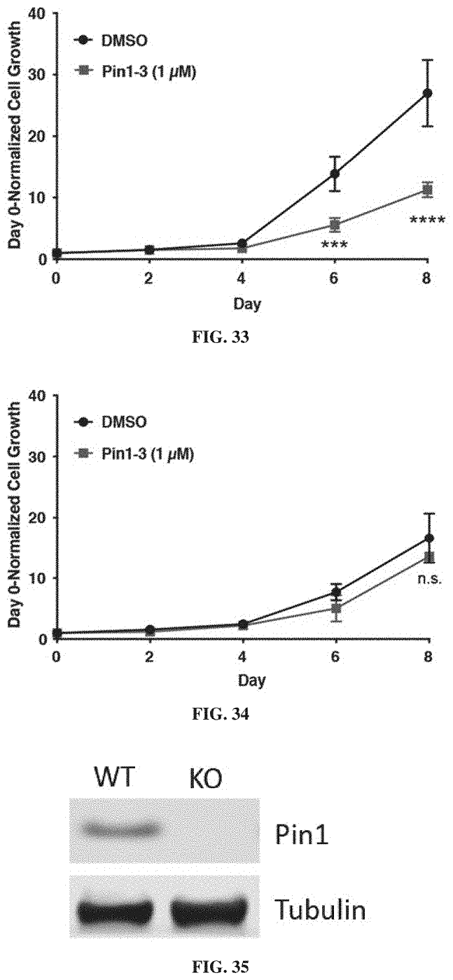

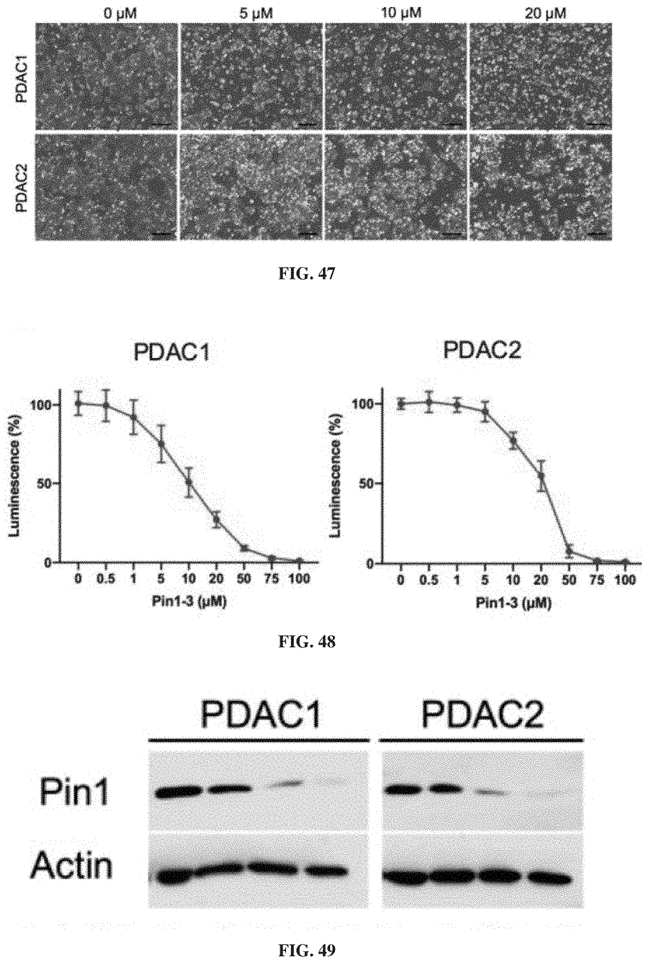

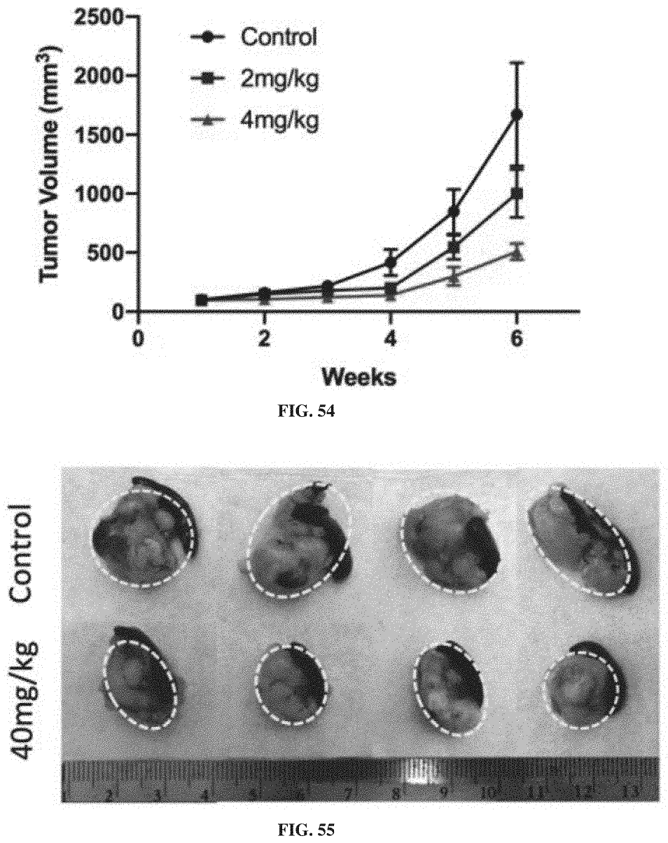

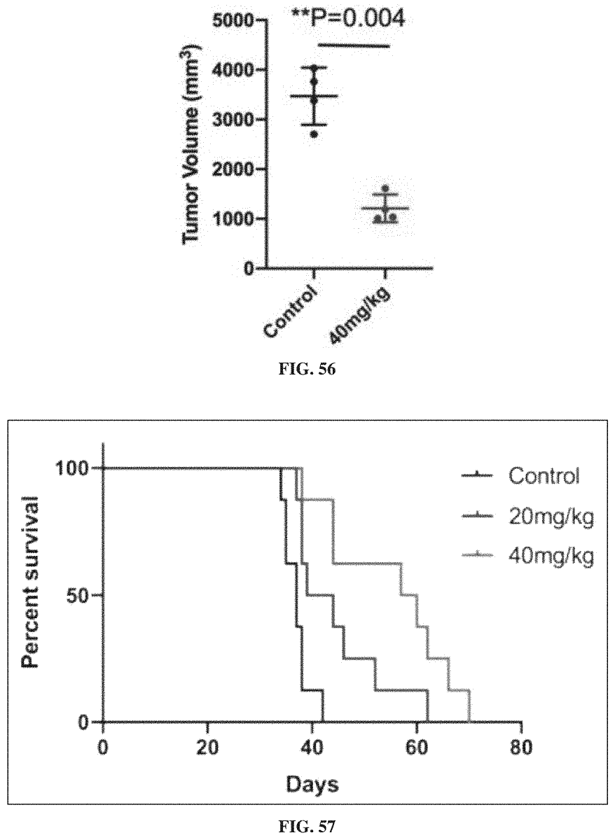

FIGS. 33-39 show that an exemplary Pin1-modulating compound inhibits growth of a variety of cancer cells, in a manner dependent on Pin1. FIGS. 47-57 show that an exemplary Pin1-modulating compound inhibits tumor growth in a variety of in vivo models.

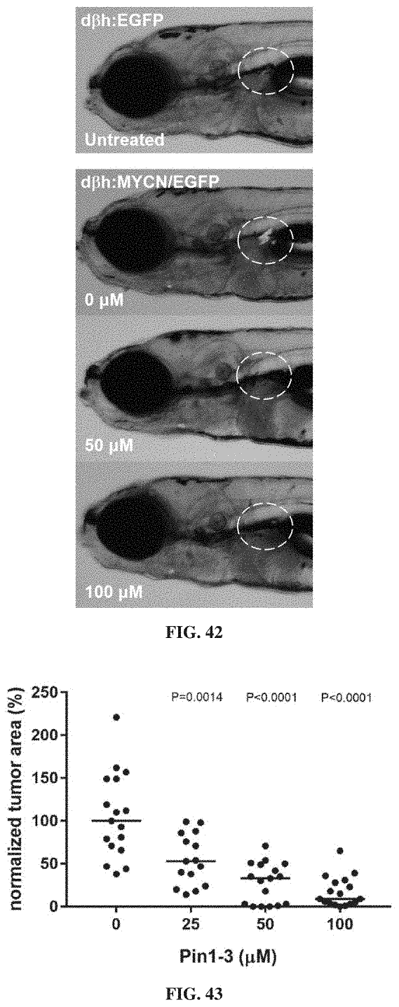

FIGS. 42-45 show that an exemplary Pin1-modulating compound inhibits initiation of neuroblastoma tumors and growth of transplanted neuroblastoma tumors.

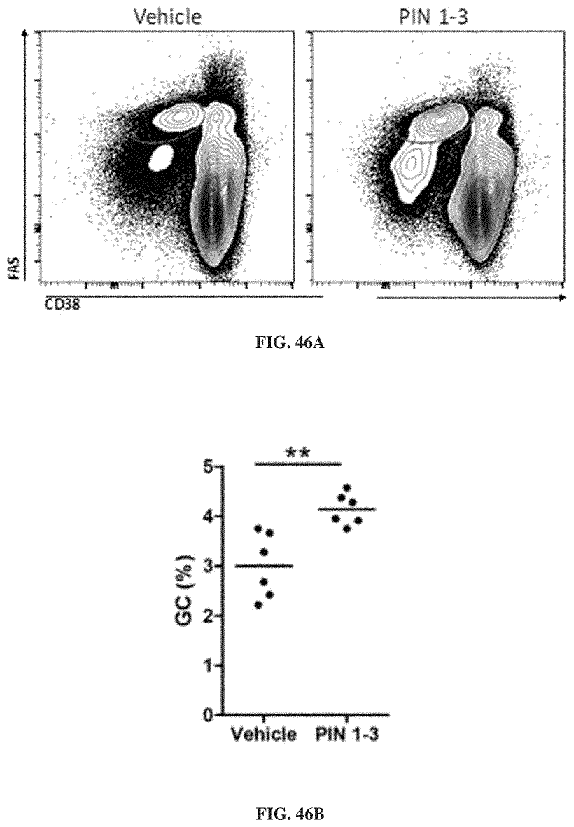

FIGS. 46A and 46B shows that Pin1 inhibition results in phenotype similar to that of Pin1-knockout.

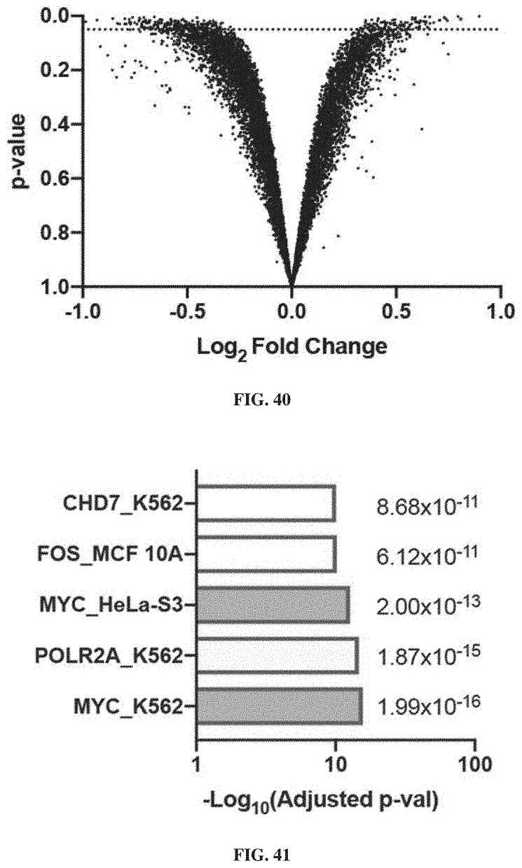

FIGS. 40-41 show that an exemplary Pin1-modulating compound inhibits Myc transcription.

Embodiments of the present invention therefore generally relate to newly designed small molecules and to uses thereof, e.g., in modulating an activity of Pin1.

Compounds:

According to some embodiments of the present invention, a compound as described herein is such that features strong association with the catalytic binding site of Pin1.

In some embodiments, the compound is such that, upon contacting the Pin1 catalytic binding site, one of its functional groups covalently binds the Cys113 residue of Pin1, and one or more other functional groups are in a proximity and orientation, as defined hereinabove, with respect to at least one another amino acid residue within the catalytic binding site of Pin1.

By “proximity and orientation” it is meant that, as discussed hereinabove, the functional group(s) are sufficiently close and properly oriented so as to strongly interact with the one or more amino acid residues (e.g., other than the Cys113) within the catalytic domain of the enzyme.

By “interacting” or “interact”, in the context of a functional group of the compound and an amino acid residue in the catalytic domain, it is meant a chemical interaction as a result of, for example, non-covalent interactions such as, but not limited to, hydrophobic interactions, including aromatic interactions, electrostatic interactions, Van der Waals interactions and hydrogen bonding. The interaction is such that results in the low dissociation constant of the compound-enzyme complex as disclosed herein.

The compounds described in some embodiments of any of the aspects of the present embodiments, and any combination thereof are characterized by electrophilic moiety and a rigid moiety that comprises at least one functional group that is capable of interacting with one or more amino acid residues in the catalytic domain of Pin1.

In some embodiments, the functional group(s) of the rigid moiety is/are capable of forming hydrogen bonds with hydrogen atoms of one or more amino acid residues in the catalytic domain of Pin1.

In some embodiments, the electrophilic moiety and the rigid moiety are arranged such that the electrophilic moiety is capable of covalently binding to the Cys113 residue of the Pin1 (SEQ ID NO: 1), and the rigid moiety is capable of forming hydrogen bonds with the Gln131 and His 157 residues of Pin1 (SEQ ID NO: 1).

In some embodiments, the compound is such that when it contacts Pin1, the functional group(s) of the rigid moiety are in proximity and orientation with respect to the electrophilic group (prior to its covalent binding to Cys113), and to amino acid residues in the catalytic domain of Pin1 (e.g., the Gln131 and His 157 residues of Pin1), e.g., via hydrogen bonding, such that the electrophilic group is in proximity and orientation with respect to Cys113, thereby facilitating covalent binding of the Cys113 to the electrophilic group.

In some embodiments, the compound is such that when it contacts Pin1, the functional group(s) of the rigid moiety are in proximity and orientation with respect to the electrophilic group after its covalent binding to Cys113, that allow interaction, e.g., via hydrogen bonding, with other amino acid residues in the catalytic domain of Pin1 (e.g., with the Gln131 and His 157 residues of Pin1).

In some embodiments, the functional group (comprised by the rigid moiety) is capable of forming a hydrogen bond with a backbone amide hydrogen of the Gln131 and/or with an imidazole NH of the His157. In some embodiments, the rigid moiety comprises a functional group capable of forming a hydrogen bond with a backbone amide hydrogen of the Gln131, and another functional group capable of forming a hydrogen bond with an imidazole NH of the His157. In some embodiments, a distance between an atom of the functional group (e.g., O, S or N) and a nitrogen atom of Gln131 or His157 linked to the functional group via a hydrogen bond is in a range of from 2.5 to 3.5 Å, optionally in a range of from 2.7 to 3.3 Å.

Herein throughout, numbering of the amino acid residues of Pin1 is in accordance with SEQ ID NO: 1.

As used herein and known in the art, a “hydrogen bond” is a relatively weak bond that forms a type of dipole-dipole attraction which occurs when a hydrogen atom bonded to a strongly electronegative atom exists in the vicinity of another electronegative atom with a lone pair of electrons.

The hydrogen atom in a hydrogen bond is partly shared between two relatively electronegative atoms.

Hydrogen bonds typically have energies of 1-3 kcal mol⁻¹ (4-13 kJ mol⁻¹), and their bond distances (measured from the hydrogen atom) typically range from 1.5 to 2.6 Å.

A hydrogen-bond donor is the group that includes both the atom to which the hydrogen is more tightly linked and the hydrogen atom itself, whereas a hydrogen-bond acceptor is the atom less tightly linked to the hydrogen atom. The relatively electronegative atom to which the hydrogen atom is covalently bonded pulls electron density away from the hydrogen atom so that it develops a partial positive charge (δ⁺). Thus, it can interact with an atom having a partial negative charge (δ⁻) through an electrostatic interaction.

Atoms that typically participate in hydrogen bond interactions, both as donors and acceptors, include oxygen, nitrogen and fluorine. These atoms typically form a part of chemical group or moiety such as, for example, carbonyl, carboxylate, amide, hydroxyl, amine, imine, alkyl fluoride, F₂, and more. However, other electronegative atoms and chemical groups or moieties containing same may participate in hydrogen bonding.

In some of any of the embodiments described herein, the compound further comprising a hydrophobic moiety, e.g., attached to the electrophilic moiety and/or to the rigid moiety. In some embodiments, the hydrophobic moiety forms a hydrophobic interaction with Ser115, Leu122 and/or Met130 of Pin1.

Herein, the term “hydrophobic moiety” refers to a moiety for which a corresponding compound (i.e., a compound consisting of the moiety and one or more hydrogen atoms attached thereto) is water-insoluble, that is, a solubility of such a compound in water is less than 1 weight percent, e.g., at room temperature (at a pH of about 7).

In some of any of the embodiments described herein, the functional moiety forming hydrogen bonds is an oxygen atom (O), a sulfur atom (S) and/or NH.

A plurality of functional moieties may optionally be the same or different, and may optionally be attached to the same position in the rigid moiety (e.g., cyclic moiety) and/or at different positions.

In some of any of the embodiments described herein, two or more functional moieties forming hydrogen bonds are attached to the same atom, for example, a sulfur atom, in the rigid moiety. In some embodiments, the functional moieties are oxygen atoms, and two oxygen atoms attached to the sulfur atom form a sulfone (—S(═O)₂—) group. In some embodiments, the sulfur atom of the sulfone is a member of a ring, that is, a cyclic sulfone (e.g., a sulfolane or sulfolene).

In some of any of the embodiments described herein, the compound has a molecular weight of less than 1000 Da. In some embodiments, the molecular weight is less than 900 Da. In some embodiments, the molecular weight is less than 800 Da. In some embodiments, the molecular weight is less than 700 Da. In some embodiments, the molecular weight is less than 600 Da. In some embodiments, the molecular weight is less than 500 Da. In some embodiments, the molecular weight is less than 400 Da.

Without being bound by any particular theory, it is believed that small molecules tend to be more promising for therapeutic use than do larger molecules.

According to some of any of the embodiments of the invention, the compound is represented by Formula I:

E-L₁-G(F)m Formula I

wherein:

E is an electrophilic moiety, according to any of the respective embodiments described herein;

L₁ is a bond or a linking moiety;

G is a rigid moiety, according to any of the respective embodiments described herein;

F is a functional moiety forming hydrogen bonds, according to any of the respective embodiments described herein; and

m is 2, 3 or 4.

In some of any of the embodiments described herein, the rigid moiety is a cyclic moiety, with 2, 3 or 4 functional moieties represented by variable F attached thereto. In some such embodiments, the cyclic moiety comprises a 4-, 5-, 6-, or 7-membered ring.

A linking moiety represented by L₁ may optionally be any linking group described herein, optionally a hydrocarbon (as defined herein).

In some exemplary embodiments, L₁ is methylene. In some exemplary embodiments, L₁ is a bond.

Herein, the phrase “linking group” describes a group (e.g., a substituent) that is attached to two or more moieties in the compound; whereas the phrase “end group” describes a group (e.g., a substituent) that is attached to a single moiety in the compound via one atom thereof.

In some of any of the embodiments described herein, m is 2, and the two functional moieties forming hydrogen bonds are attached to the same atom, for example, a sulfur atom, in the rigid moiety (according to any of the respective embodiments described herein), for example, wherein the rigid moiety comprises a sulfone (e.g., a sulfolane or sulfolene).

In some of any of the embodiments described herein, the rigid moiety is a cyclic moiety comprising a sulfur atom, and the compound is represented by Formula Ia:

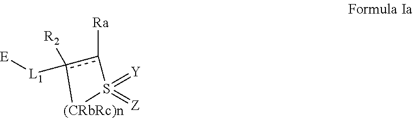

wherein:

E and L₁ are as defined herein for Formula I;

the dashed line represents a saturated or non-saturated bond;

Y and Z are each independently O, S and/or NH (according to any of the respective embodiments described herein with respect to variable F in Formula I);

R₂ and Ra-Rc are each independently hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and/or amino, or alternatively, R₂ is absent when the dashed line represents an unsaturated bond; and

n is 1, 2, 3 or 4, such that there are 1, 2, 3 or 4 units of CRbRc (forming a 4-, 5-, 6- or 7-membered ring, respectively), and when n is 2 or more, the 2 or more units may be the same or different.

In exemplary embodiments, n is 2.

In some of any of the respective embodiments described herein, Y and Z are each oxygen, thus forming a cyclic sulfone. In some such embodiments, n is 2 such that the cyclic sulfone is a sulfolane or sulfolene.

In some of any of the respective embodiments described herein, Ra is hydrogen.

In some of any of the respective embodiments described herein, Rb is hydrogen. In some embodiments, Rb and Rc are each hydrogen. In some embodiments, Ra, Rb and Rc are each hydrogen.

In some of any of the respective embodiments described herein, the dashed line represents a saturated bond.

In some of any of the respective embodiments described herein, R₂ is hydrogen or alkyl. In some embodiments, R₂ is hydrogen or C₁₋₄-alkyl. In some embodiments, R₂ is hydrogen or methyl. In some embodiments, R₂ is hydrogen.

Herein, the terms “electrophile” and “electrophilic moiety” refer to any moiety capable of reacting with a nucleophile (e.g., a moiety having a lone pair of electrons, a negative charge, a partial negative charge and/or an excess of electrons, for example a thiol group). Electrophilic moieties typically are electron poor or comprise atoms which are electron poor.

In some of any of the respective certain embodiments, an electrophilic moiety contains a positive charge or partial positive charge, has a resonance structure which contains a positive charge or partial positive charge or is a moiety in which delocalization or polarization of electrons results in one or more atom which contains a positive charge or partial positive charge. In some embodiments, the electrophilic moiety comprises conjugated double bonds, for example, an α,β-unsaturated carbonyl.

The electrophilic moiety may optionally be capable of binding to a sulfur atom of the Cys113, for example, by nucleophilic substitution (e.g., of a nucleophilic leaving group) and/or by Michael addition, e.g., to a carbon-carbon unsaturated bond, optionally activated by an adjacent C═O (e.g., of carbonyl, C-carboxy or C-amido) or nitro group.

A “leaving group” as used herein and in the art describes a labile atom, group or chemical moiety that readily undergoes detachment from an organic molecule during a chemical reaction, while the detachment is typically facilitated by the relative stability of the leaving atom, group or moiety thereupon.

Typically, any group that is the conjugate base of a strong acid can act as a leaving group. For example, a suitable nucleophilic leaving groups may optionally be any group which, when attached to a hydrogen atom, forms an acid having a pKa of less than 7. Examples of suitable leaving groups include, without limitation, halide (halo, preferably chloro, bromo or iodo), sulfate, sulfonate (e.g., tosylate or triflate), trichloroacetimidate, azide, cyanate, thiocyanate, nitrate and O-carboxy (e.g., acetate).

In some of any of the respective embodiments, the nucleophilic leaving group, when attached to a hydrogen atom, forms an acid having a pKa of less than 0, e.g., iodo, bromo, chloro, sulfate or sulfonate.

In some of any of the respective embodiments, the electrophilic moiety comprises halo, optionally bromo, chloro or fluoro. In some embodiments, the electrophilic moiety comprises a haloalkyl group (i.e., alkyl, as defined herein, substituted with halo). In some embodiments, the haloalkyl is substituted by halo (e.g., chloro or fluoro) at a terminal position thereof (i.e., primary carbon), for example, wherein the haloalkyl is halomethyl (e.g., chloromethyl or fluoromethyl). Chloromethyl is an exemplary haloalkyl group.

In some of any of the respective embodiments, the electrophilic moiety has a formula —NR₁—C(═W)-L₂-X, wherein W is O, S and/or NR₃; X is halo; L₂ is alkylene; and R₁ and R₃ are each independently hydrogen, alkyl, alkenyl, alkynyl, cycloalkyl, heteroalicyclic, aryl and/or heteroaryl. In some embodiments, R₁ is a hydrophobic moiety according to any of the respective embodiments described herein. In some embodiments, W is O.

In some of any of the respective embodiments, the electrophilic moiety comprises a haloacetamide, that is, a derivative of acetamide (—NH—C(═O)—CH₃) which is substituted by halo, and optionally by any other suitable substituent defined herein (an alkyl substituent at the CH₃ group and/or an amide substituent at the amide nitrogen atom), e.g., wherein L₂ (as defined herein) is substituted or unsubstituted methylene. In exemplary embodiments, the haloacetamide comprises a single halo and no additional substituent at the CH₃ group, thereby having a formula —NR₁—C(═O)—CH₂X, wherein X is halo (e.g., chloro), and R₁ is as defined herein.

In some of any of the respective embodiments, the electrophilic moiety comprises a substituted or unsubstituted acryloyl group, i.e., an acryloyl (—CH═CH—C(═O)—) group or substituted derivative thereof, which may optionally be in a form of an ester (e.g., the electrophilic moiety having a formula —O—C(═O)—CH═CH₂) or amide (e.g., having a formula —NR—C(═O)—CH═CH₂, wherein R is a suitable substituent of an amide group as defined herein). A substituted acryloyl is optionally a cyanoacryloyl (substituted by cyano the position proximal to the C═O, i.e., the α position). Alternatively or additionally, the acryloyl is substituted by alkyl (e.g., C₁₋₄-alkyl), at the α or β position.

In some of any of the embodiments relating to an electrophilic moiety comprising an acryloyl group, the group is an unsubstituted (meth)acryloyl group, i.e., an acryloyl (—CH═CH—C(═O)—) or methacryloyl (—CH═C(CH₃)—C(═O)—) group, which may optionally be in a form of a (meth)acrylate ester or (meth)acrylamide.

In some of any of the respective embodiments, the electrophilic moiety comprises a substituted or unsubstituted vinylsulfonyl group, i.e., a —S(═O)₂—CH═CH₂ or substituted derivative thereof, which may optionally be in a form of a sulfonate ester (e.g., the electrophilic moiety having a formula —O—S(═O)₂)—CH═CH₂ or sulfonamide (e.g., having a formula —NR—S(═O)₂—CH═CH₂, wherein R is a suitable substituent of a sulfonamide group as defined herein).

In some of any of the respective embodiments, the electrophilic moiety comprises an α-ketoamide, i.e., including a —NR—C(═O)—C(═O)— linking group (wherein R is a suitable substituent of an amide group as defined herein).

Additional examples of suitable electrophilic moieties which may be incorporated in compounds described herein are described in U.S. Pat. Nos. 9,227,978 and 7,514,444, the contents of each of which are incorporated herein by reference, particularly contents describing electrophilic moieties.

It is to be appreciated that an amide linking group (as defined herein) can provide a strong (and readily formed) covalent bond between the electrophilic moiety and the rigid moiety, according to any of the respective embodiments described herein, and may optionally provide an additional covalent bond to a suitable moiety (e.g., a hydrophobic moiety, according to any of the respective embodiments described herein) which may further enhance affinity to Pin1, e.g., a moiety represented herein by the variable R₁ (according to any of the respective embodiments described herein).

In some of any of embodiments described herein wherein the electrophilic moiety has a formula —NR₁—C(═W)-L₂-X, the compound is represented by Formula Ia, such that the compound is represented by Formula Ib:

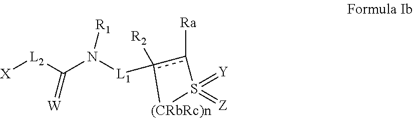

wherein W is O, S and/or NR₃; X is halo; Ra-Rc are optionally each hydrogen; L₁ is a bond or alkylene; L₂ is alkylene; and R₁ and R₃ are each independently hydrogen, alkyl, alkenyl, alkynyl, cycloalkyl, heteroalicyclic, aryl and/or heteroaryl.

In some of any of the embodiments described herein, the rigid moiety is a sulfolane or sulfolene moiety (according to any of the respective embodiments described herein), comprising two oxygen atoms as functional groups capable of forming hydrogen bonds, and the electrophilic moiety is a haloacetamide (according to any of the respective embodiments described herein). In some such embodiments, the compound is represented by Formula Ic:

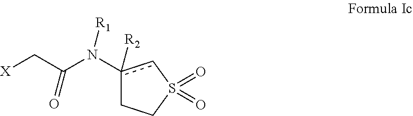

wherein the dashed line represents a saturated or non-saturated bond; X is halo; and R₁ and R₂ are as defined herein according to any of the respective embodiments. In exemplary embodiments, X is chloro.

In some of any of the respective embodiments described herein, R₁ is an alkyl, alkenyl or alkynyl having Formula II:

—CH₂—R′₁ Formula II

wherein R′₁ is alkenyl (such that R₁ as a whole is an alkenyl), alkynyl (such that R₁ as a whole is an alkynyl), alkyl (such that R₁ as a whole is a substituted or unsubstituted alkyl), or cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, or amino (such that R₁ as a whole is a substituted alkyl).

Without being bound by any particular theory, it is believed that the unsubstituted methylene (CH₂) adjacent to the nitrogen atom (to which R₁ is attached) enhances binding of the compound to Pin1.

In some of any of the respective embodiments, R′₁ is a branched alkyl, branched alkenyl, branched alkynyl, cycloalkyl or heteroalicyclic. In some embodiments, R′₁ is a secondary alkyl, alkenyl, alkynyl, cycloalkyl or heteroalicyclic, that is, a carbon atom of R′₁ proximal to the CH₂ (depicted in Formula II) is attached to two other carbon atoms in R′₁. In some embodiments, R′₁ is a tertiary alkyl, alkenyl, alkynyl, cycloalkyl or heteroalicyclic, that is, a carbon atom of R′₁ proximal to the CH₂ (depicted in Formula II) is attached to three other carbon atoms in R′₁. Exemplary tertiary R′₁ groups include (substituted or unsubstituted) t-butyl (e.g., as in exemplary compounds Pin1-3 and Pin1-3-DTB); and 1-trifluoromethyl cyclopropyl (e.g., as in exemplary compound Pin1-3-9), a tertiary cycloalkyl group.

In some of any of the respective embodiments, R₁ or R′₁ is aryl, for example, wherein R′₁ is aryl (and R₁ is —CH₂-aryl). In some embodiments, the aryl is a phenyl, which may be unsubstituted or substituted, for example, by alkyl (e.g., methyl), halo (e.g., fluoro or chloro), aryl (e.g., phenyl or 3-triflluoromethylphenyl) and/or alkoxy (e.g., benzyloxy). Exemplary phenyls include unsubstituted phenyl (e.g., as in exemplary compounds Pin1-437 and Pin1-2-9), m-methylphenyl (e.g., as in exemplary compound Pin1-2-6), and o-benzyloxyphenyl (e.g., as in exemplary compound Pin1-2-7).

In some of any of the respective embodiments, R₁ or R′₁ is heteroaryl, for example, wherein R′₁ is heteroaryl (and R₁ is —CH₂-heteroaryl).

In some embodiments, the heteroaryl is a triazole, thiophene (e.g., a thiophen-2-yl) or furan (e.g., a furan-2-yl), each of which may be substituted or unsubstituted.

In some embodiments, the heteroaryl is a thiophene (e.g., thiophen-2-yl or 3-methyl-thiophen-2-yl, as in exemplary compounds Pin1-433 and Pin1-2-8, respectively).

In some embodiments, the heteroaryl is a (substituted or unsubstituted) triazole, which may optionally have Formula III:

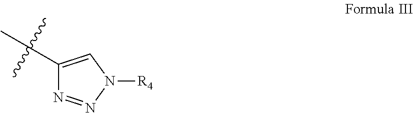

wherein R₄ is alkyl, alkenyl, alkynyl, cycloalkyl, heteroalicyclic, aryl or heteroaryl.

In some of any of the respective embodiments, the heteroaryl is substituted by one or more (substituted or unsubstituted) phenyl, for example, wherein R₄ in Formula III is a phenyl. The phenyl substituent may optionally be substituted, for example, by one or more hydroxy, hydroxyalkyl (e.g., hydroxymethyl or hydroxyethyl), halo (e.g., fluoro, chloro or bromo), alkoxy (e.g., methoxy or ethoxy), carbonyl (e.g., formyl or acetyl), carboxy (e.g., a C-carboxy ester group, such as methoxycarbonyl or ethoxycarbonyl), and/or sulfonamido (e.g., —S(═O)₂NH₂).

The phenyl substituent (according to any of the respective embodiments) may optionally be substituted at an ortho position thereof (e.g., by hydroxy), at a meta position thereof (e.g., by halo or carbonyl), and/or at a para position thereof, for example, by hydroxy, hydroxyalkyl (e.g., hydroxymethyl), alkoxy (e.g., methoxy), carbonyl (e.g., acetyl), carboxy (e.g., methoxycarbonyl) or sulfonamido (e.g., —S(═O)₂NH₂). In some exemplary embodiments (e.g., in exemplary compound P1-01-B11) the phenyl is p-methoxycarbonylphenyl.

In some embodiments, there is provided a compound represented by Formula Ib, wherein W, X, Y, Z, Ra-Rc, L₁, L₂, n, R₂ and R₃ are as described according to any of the respective embodiments described herein, and R₁ is an isobutyl (e.g., —CH₂—CH(CH₃)₂), a neopentyl (e.g., —CH₂—C(CH₃)₃), an alkyl (e.g., methyl) substituted by a 5- or 6-membered cycloalkyl, an alkyl (e.g., methyl) substituted by a triazole, or a triazole (according to any of the respective embodiments described herein). Such structures wherein an R₁ group is defined in such a manner are also referred to herein as Formula Id.

Exemplary cycloalkyl groups according to Formula Id include unsubstituted cyclopentyl and unsubstituted cycloalkyl.

In some of any of the respective embodiments relating to Formula Id, R₁ is a neopentyl (e.g., —CH₂—C(CH₃)₃), an alkyl (e.g., methyl) substituted by a triazole, or a triazole (according to any of the respective embodiments described herein). In exemplary embodiments, R₁ is a neopentyl (e.g., —CH₂—C(CH₃)₃) or an alkyl (e.g., methyl) substituted by a triazole (according to any of the respective embodiments described herein).

As exemplified in the Examples section herein, compounds of Formula Id may be readily prepared (e.g., from commonly available precursors) using click chemistry to form a triazole (from an alkynyl precursor, which may be commercially available) or using an aldehyde under reducing conditions to form an (optionally substituted) alkyl group.

Libraries:

According to an aspect of some embodiments of the invention, there is provided a screening library comprising a plurality of compounds according to any of the embodiments described herein, for example, a plurality of compounds according to Formula I, a plurality of compounds according to Formula Ia, a plurality of compounds according to Formula Ib, a plurality of compounds according to Formula Ic, and/or a plurality of compounds according to Formula Id.

According to an aspect of some embodiments of the invention, there is provided a method of identifying a compound capable of modulating an activity of Pin1 (according to any of the respective embodiments described herein). The method comprises screening a plurality of compounds represented by Formula IV:

E′-L′₁-V Formula IV

wherein E′ is an electrophilic moiety capable of forming a covalent bond when reacted with a thiol according to any of the respective embodiments described herein; L′₁ is a linking moiety according to any of the respective embodiments described herein (e.g., with respect to L₁); and V is a moiety featuring at least two functional groups that are capable of forming hydrogen bonds, and optionally further features at least one lipophilic group (according to any of the respective embodiments described herein).

In some embodiments, the screening is for compounds that are capable of interacting with a Cys113 residue of Pin1 via the electrophilic moiety, of interacting at least with the Gln131 and His 157 residues of Pin1 via the functional groups, and optionally of interacting with at least one amino acid residue in a hydrophobic patch of Pin1 via the at least one lipophilic group. A compound identified as capable of interacting at least with the Cys113 residue and the Gln131 and His 157 residues of Pin1 is identified as capable of modifying an activity of Pin1.

Screening may optionally be effected by computational docking (e.g., as exemplified herein).

Alternatively or additionally, screening may optionally be effected by contacting the identified compound with Pin1, to thereby determine if the compound binds (e.g., covalently) to Pin1 and/or modulate an activity of Pin1. A compound may be identified as capable of modifying an activity of Pin1 by direct determination of a capability of such modulation, and/or less directly, wherein a compound that is determined as capable of binding (e.g., covalently) to Pin1 is identified as capable of modulating an activity of Pin1.

In some embodiments, the method comprises screening a plurality of compounds according to Formula I, a plurality of compounds according to Formula Ia, a plurality of compounds according to Formula Ib, a plurality of compounds according to Formula Ic, and/or a plurality of compounds according to Formula Id, with Pin1 under conditions that allow covalent binding of a Cys113 residue of Pin1 to an electrophilic moiety described herein, optionally by nucleophilic substitution of a halo atom in an electrophilic moiety by Cys113.

Suitable conditions for covalent binding of a Cys113 residue to an electrophilic moiety may be as exemplified herein, e.g., in an aqueous solution (e.g., buffered at pH 7.4) at room temperature or under refrigeration (e.g., 4° C.).

In some of any of the embodiments relating to a method of identifying a compound capable of modulating an activity of Pin1, the method further comprises screening the library for low reactivity with a thiol other than Cys113 of Pin1.

In exemplary embodiments, reactivity with a thiol is determined by adding a compound (e.g., at a concentration of 200 μM) to an aqueous solution (e.g., buffered at pH 7.4) of thionitrobenzoate (TNB²⁻) (e.g., at 37° C.), optionally at a concentration of 100 μM TNB²⁻; determining absorbance of the TNB²⁻ over time (e.g., at about 412 nm); and fitting the spectroscopic data to a second order reaction equation such that the rate constant k is the slope of ln([A][B₀]/[B][A₀]), where [A₀] and [B₀] are the initial concentrations of the compound (e.g., 200 μM) and TNB²⁻ (e.g., 100 μM) respectively, and [A] and [B] are the remaining concentrations as a function of time compounds.

In some embodiments, a compound exhibiting low reactivity with a thiol is a compound for which the rate constant k is no more than 3×10⁻⁷ M⁻¹*second⁻¹. In some embodiments, the rate constant k is no more than 2×10⁻⁷ M⁻¹*second⁻¹. In some embodiments, the rate constant k is no more than 10⁻⁷ M⁻¹*second⁻¹. In some embodiments, the rate constant k is no more than 5×10⁻⁸ M⁻¹*second⁻¹. In some embodiments, the rate constant k is no more than 3×10⁻⁸ M⁻¹*second⁻¹. In some embodiments, the rate constant k is no more than 2×10⁻⁸ M⁻¹*second⁻¹. In some embodiments, the rate constant k is no more than 10⁻⁸ M⁻¹*second⁻¹. In some embodiments, the rate constant k is no more than 5×10⁻⁹ M⁻¹*second⁻¹.

In some of any of the respective embodiments, according to any of the aspects described herein, the plurality of compounds comprises at least 30 distinct compounds. In some embodiments, the library comprises at least 50 compounds. In some embodiments, the library comprises at least 100 compounds. In some embodiments, the library comprises at least 200 compounds. In some embodiments, the library comprises at least 300 compounds. In some embodiments, the library comprises at least 500 compounds.

The skilled person will be capable of selecting a suitable library depending on desired property of the library as a whole. For example, library compounds encompassed by a relatively narrow formula (e.g., Formula Ib, Formula Ic and/or Formula Id) may provide a relatively high proportion of hits (as the formulas were designed for this purpose), but may suffer from relatively low internal diversity; whereas library compounds encompassed only by a relatively broad formula (e.g., Formula I, Formula Ia and/or Formula IV) may provide a relatively high internal diversity, at the expense of the proportion of hits.

Indications and Uses:

The compound(s) according to any of the embodiments described herein may optionally be for use in treating a condition in which modulating an activity of Pin1 is beneficial.

It is expected that during the life of a patent maturing from this application many relevant conditions will be identified and the scope of the term “condition in which modulating an activity of Pin1 is beneficial” is intended to include all such new treatment types a priori.

According to an aspect of some embodiments of the invention, there is provided a use of one or more compounds according to any of the embodiments described herein in the manufacture of a medicament for treating a condition in which modulating an activity of Pin1 is beneficial.

According to an aspect of some embodiments of the invention, there is provided a method of treating a condition in which modulating an activity of Pin1 is beneficial, the method comprising administering to a subject in need thereof one or more compounds according to any of the embodiments described herein.

According to an aspect of some embodiments of the invention, there is provided a method of modulating an activity of Pin1, the method comprising contacting the Pin1 with one or more compounds according to any of the embodiments described herein. Modulation of Pin1 activity may optionally be effected in vitro (e.g., for research purposes) or in vivo (e.g., wherein contacting is effected by administration to a subject in need thereof).

Herein, the term “modulation” encompasses up-regulation as well as down-regulation (e.g., by antagonistic binding) of an activity (e.g., of Pin1), and may be effected, e.g., by interacting with an active site (e.g., of Pin1) or by modulating degradation of the protein.

In some of any of the respective embodiments described herein, according to any of the aspects described herein, modulating an activity of Pin1 comprises inhibiting an activity of Pin1.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology.

Examples of conditions in which modulating an activity of Pin1 may be beneficial include, without limitation, proliferative diseases or disorders and immune diseases or disorders. The proliferative disease or disorder may be, for example, a cancer or pre-cancer.

In some of any of the respective embodiments described herein, treatment is for inhibiting initiation of a tumor (optionally neuroblastoma), for example, inhibiting metastases.

Non-limiting examples of Pin1-associated cancers which can be treated according to some of the respective embodiments of the invention can be any solid or non-solid cancer and/or cancer metastasis, including, but is not limiting to, tumors of the gastrointestinal tract (colon carcinoma, rectal carcinoma, colorectal carcinoma, colorectal cancer, colorectal adenoma, hereditary nonpolyposis type 1, hereditary nonpolyposis type 2, hereditary nonpolyposis type 3, hereditary nonpolyposis type 6; colorectal cancer, hereditary nonpolyposis type 7, small and/or large bowel carcinoma, esophageal carcinoma, tylosis with esophageal cancer, stomach carcinoma, pancreatic carcinoma, pancreatic endocrine tumors), endometrial carcinoma, dermatofibrosarcoma protuberans, gallbladder carcinoma, Biliary tract tumors, prostate cancer, prostate adenocarcinoma, renal cancer (e.g., Wilms' tumor type 2 or type 1), liver cancer (e.g., hepatoblastoma, hepatocellular carcinoma, hepatocellular cancer), bladder cancer, embryonal rhabdomyosarcoma, germ cell tumor, trophoblastic tumor, testicular germ cells tumor, immature teratoma of ovary, uterine, epithelial ovarian, sacrococcygeal tumor, choriocarcinoma, placental site trophoblastic tumor, epithelial adult tumor, ovarian carcinoma, serous ovarian cancer, ovarian sex cord tumors, cervical carcinoma, uterine cervix carcinoma, small-cell and non-small cell lung carcinoma, nasopharyngeal, breast carcinoma (e.g., ductal breast cancer, invasive intraductal breast cancer, sporadic; breast cancer, susceptibility to breast cancer, type 4 breast cancer, breast cancer-1, breast cancer-3; breast-ovarian cancer), squamous cell carcinoma (e.g., in head and neck), neurogenic tumor, astrocytoma, ganglioblastoma, neuroblastoma, lymphomas (e.g., Hodgkin's disease, non-Hodgkin's lymphoma, B cell, Burkitt, cutaneous T cell, histiocytic, lymphoblastic, T cell, thymic), gliomas, adenocarcinoma, adrenal tumor, hereditary adrenocortical carcinoma, brain malignancy (tumor), various other carcinomas (e.g., bronchogenic large cell, ductal, Ehrlich-Lettre ascites, epidermoid, large cell, Lewis lung, medullary, mucoepidermoid, oat cell, small cell, spindle cell, spinocellular, transitional cell, undifferentiated, carcinosarcoma, choriocarcinoma, cystadenocarcinoma), ependimoblastoma, epithelioma, erythroleukemia (e.g., Friend, lymphoblast), fibrosarcoma, giant cell tumor, glial tumor, glioblastoma (e.g., multiforme, astrocytoma), glioma hepatoma, heterohybridoma, heteromyeloma, histiocytoma, hybridoma (e.g., B cell), hypernephroma, insulinoma, islet tumor, keratoma, leiomyoblastoma, leiomyosarcoma, leukemia (e.g., acute lymphatic, acute lymphoblastic, acute lymphoblastic pre-B cell, acute lymphoblastic T cell leukemia, acute—megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia), lymphosarcoma, melanoma, mammary tumor, mastocytoma, medulloblastoma, mesothelioma, metastatic tumor, monocyte tumor, multiple myeloma, myelodysplastic syndrome, myeloma, nephroblastoma, nervous tissue glial tumor, nervous tissue neuronal tumor, neurinoma, neuroblastoma, oligodendroglioma, osteochondroma, osteomyeloma, osteosarcoma (e.g., Ewing's), papilloma, transitional cell, pheochromocytoma, pituitary tumor (invasive), plasmacytoma, retinoblastoma, rhabdomyosarcoma, sarcoma (e.g., Ewing's, histiocytic cell, Jensen, osteogenic, reticulum cell), schwannoma, subcutaneous tumor, teratocarcinoma (e.g., pluripotent), teratoma, testicular tumor, thymoma and trichoepithelioma, gastric cancer, fibrosarcoma, glioblastoma multiforme; multiple glomus tumors, Li-Fraumeni syndrome, liposarcoma, lynch cancer family syndrome II, male germ cell tumor, mast cell leukemia, medullary thyroid, multiple meningioma, endocrine neoplasia myxosarcoma, paraganglioma, familial nonchromaffin, pilomatricoma, papillary, familial and sporadic, rhabdoid predisposition syndrome, familial, rhabdoid tumors, soft tissue sarcoma, and Turcot syndrome with glioblastoma.

Pancreatic cancer (e.g., pancreatic adenocarcinoma) is an exemplary type of cancer treatable according to some embodiments of the invention.

Pre-cancers are well characterized and known in the art (refer, for example, to Berman J J. and Henson D E., 2003. Classifying the precancers: a metadata approach. BMC Med Inform Decis Mak. 3:8). Classes of pre-cancers amenable to treatment via the method of the invention include acquired small or microscopic pre-cancers, acquired large lesions with nuclear atypia, precursor lesions occurring with inherited hyperplastic syndromes that progress to cancer, and acquired diffuse hyperplasias and diffuse metaplasias. Examples of small or microscopic pre-cancers include HGSIL (High grade squamous intraepithelial lesion of uterine cervix), AIN (anal intraepithelial neoplasia), dysplasia of vocal cord, aberrant crypts (of colon), PIN (prostatic intraepithelial neoplasia). Examples of acquired large lesions with nuclear atypia include tubular adenoma, AILD (angioimmunoblastic lymphadenopathy with dysproteinemia), atypical meningioma, gastric polyp, large plaque parapsoriasis, myelodysplasia, papillary transitional cell carcinoma in-situ, refractory anemia with excess blasts, and Schneiderian papilloma. Examples of precursor lesions occurring with inherited hyperplastic syndromes that progress to cancer include atypical mole syndrome, C cell adenomatosis and MEA. Examples of acquired diffuse hyperplasias and diffuse metaplasias include AIDS, atypical lymphoid hyperplasia, Paget's disease of bone, post-transplant lymphoproliferative disease and ulcerative colitis.

Therapeutic regimens for treatment of cancer suitable for combination with one or more compounds according to any of the respective embodiments of the invention include, but are not limited to chemotherapy, radiotherapy, phototherapy and photodynamic therapy, surgery, nutritional therapy, ablative therapy, combined radiotherapy and chemotherapy, brachiotherapy, proton beam therapy, immunotherapy, cellular therapy and photon beam radiosurgical therapy.

Alternative or additional chemotherapeutic drugs (e.g., anti-cancer drugs) that may optionally be co-administered with compounds of the invention include, but are not limited to acivicin, aclarubicin, acodazole, acronine, adozelesin, aldesleukin, altretamine, ambomycin, ametantrone, aminoglutethimide, amsacrine, anastrozole, anthramycin, asparaginase, asperlin, azacitidine, azetepa, azotomycin, batimastat, benzodepa, bicalutamide, bisantrene, bisnafide, bizelesin, bleomycin, brequinar, bropirimine, busulfan, cactinomycin, calusterone, caracemide, carbetimer, carboplatin, carmustine, carubicin, carzelesin, cedefingol, chlorambucil, cirolemycin, cisplatin, cladribine, crisnatol, cyclophosphamide, cytarabine, dacarbazine, dactinomycin, daunorubicin, decitabine, dexormaplatin, dezaguanine, diaziquone, docetaxel, doxorubicin, droloxifene, dromostanolone, duazomycin, edatrexate, eflornithine, elsamitrucin, enloplatin, enpromate, epipropidine, epirubicin, erbulozole, esorubicin, estramustine, etanidazole, etoposide, etoprine, fadrozole, fazarabine, fenretinide, floxuridine, fludarabine, fluorouracil, flurocitabine, fosquidone, fostriecin, gemcitabine, hydroxyurea, idarubicin, ifosfamide, ilmofosine, interferon alfa-2a, interferon alfa-2b, interferon alfa-nl, interferon alfa-n3, interferon beta-Ia, interferon gamma-Ib, iproplatin, irinotecan, lanreotide, letrozole, leuprolide, liarozole, lometrexol, lomustine, losoxantrone, masoprocol, maytansine, mechlorethamine, megestrol, melengestrol, melphalan, menogaril, mercaptopurine, methotrexate, metoprine, meturedepa, mitindomide, mitocarcin, mitocromin, mitogillin, mitomalcin, mitomycin, mitosper, mitotane, mitoxantrone, mycophenolic acid, nocodazole, nogalamycin, ormaplatin, oxisuran, paclitaxel, pegaspargase, peliomycin, pentamustine, peplomycin, perfosfamide, pipobroman, piposulfan, piroxantrone, plicamycin, plomestane, porfimer, porfiromycin, prednimustine, procarbazine, puromycin, pyrazofurin, riboprine, rogletimide, safingol, semustine, simtrazene, sparfosate, sparsomycin, spirogermanium, spiromustine, spiroplatin, streptonigrin, streptozocin, sulofenur, talisomycin, tecogalan, tegafur, teloxantrone, temoporfin, teniposide, teroxirone, testolactone, thiamiprine, thioguanine, thiotepa, tiazofurin, tirapazamine, topotecan, toremifene, trestolone, triciribine, trimetrexate, triptorelin, tubulozole, uracil mustard, uredepa, vapreotide, verteporfin, vinblastine, vincristine, vindesine, vinepidine, vinglycinate, vinleurosine, vinorelbine, vinrosidine, vinzolidine, vorozole, zeniplatin, zinostatin, zorubicin, and any pharmaceutically acceptable salts thereof. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

It is expected that during the life of a patent maturing from this application many relevant drugs will be developed and the scope of the terms “anti-cancer agent”, “chemotherapeutic drug”, “antineoplastic agent” and the like are intended to include all such new technologies a priori.

Additional anti-cancer agents may optionally be selected in accordance with the condition to be treated, for example, by selecting an agent for use in treating a condition for which the agent (per se) has already been approved, e.g., as indicated in the following table:

| | | |
|---|---|---|
| Aldesleukin | Proleukin | |
| Alemtuzumab | Campath | Accel. Approv. (clinical benefit not established) Campath is |
| | | indicated for the treatment of B-cell chronic lymphocytic leukemia |
| | | (B-CLL) in patients who have been treated with alkylating agents |
| | | and who have failed fludarabine therapy. |
| alitretinoin | Panretin | Topical treatment of cutaneous lesions in patients with AIDS-related |
| | | Kaposi's sarcoma. |
| allopurinol | Zyloprim | Patients with leukemia, lymphoma and solid tumor malignancies |
| | | Who are receiving cancer therapy which causes elevations of serum |
| | | and urinary uric acid levels and who cannot tolerate oral therapy. |
| altretamine | Hexalen | Single agent palliative treatment of patients with persistent or |
| | | recurrent ovarian cancer following first-line therapy with a cisplatin |
| | | and/or alkylating agent based combination. |
| amifostine | Ethyol | To reduce the cumulative renal toxicity associated with repeated |
| | | administration of cisplatin in patients with advanced ovarian cancer |
| amifostine | Ethyol | Accel. Approv. (clinical benefit not established) Reduction of |
| | | platinum toxicity in non-small cell lung cancer |
| amifostine | Ethyol | To reduce post-radiation xerostomia for head and neck cancer |
| | | where the radiation port includes a substantial portion of the parotid |
| | | glands. |
| anastrozole | Arimidex | Accel. Approv. (clinical benefit not established) for the adjuvant |
| | | treatment of postmenopausal women with hormone receptor |
| | | positive early breast cancer |
| anastrozole | Arimidex | Treatment of advanced breast cancer in postmenopausal women |
| | | with disease progression following tamoxifen therapy. |
| anastrozole | Arimidex | For first-line treatment of postmenopausal women with hormone |
| | | receptor positive or hormone receptor unknown locally advanced or |
| | | metastatic breast cancer. |
| arsenic trioxide | Trisenox | Second line treatment of relapsed or refractory APL following ATRA |
| | | plus an anthracycline. |
| Asparaginase | Elspar | ELSPAR is indicated in the therapy of patients with acute |
| | | lymphocytic leukemia. This agent is useful primarily in combination |
| | | with other chemotherapeutic agents in the induction of remissions |
| | | of the disease in pediatric patients. |
| BCG Live | TICE BCG | |
| bexarotene capsules | Targretin | For the treatment by oral capsule of cutaneous manifestations of |
| | | cutaneous T-cell lymphoma in patients who are refractory to at least |
| | | one prior systemic therapy. |
| bexarotene gel | Targretin | For the topical treatment of cutaneous manifestations of cutaneous |
| | | T-cell lymphoma in patients who are refractory to at least one prior |
| | | systemic therapy. |
| bleomycin | Blenoxane | Sclerosing agent for the treatment of malignant pleural effusion |
| | | MPE) and prevention of recurrent pleural effusions. |
| busulfan intravenous | Busulfex | Use in combination with cyclophoshamide as conditioning regimen |
| | | prior to allogeneic hematopoietic progenitor cell transplantation for |
| | | chronic myelogenous leukemia. |
| busulfan oral | Myleran | Chronic Myelogenous Leukemia- palliative therapy |
| calusterone | Methosarb | |
| capecitabine | Xeloda | Accel. Approv. (clinical benefit subsequently established) |
| | | Treatment of metastatic breast cancer resistant to both paclitaxel |
| | | and an anthracycline containing chemotherapy regimen or resistant |
| | | to paclitaxel and for whom further anthracycline therapy may be |
| | | contraindicated, e.g., patients who have received cumulative doses |
| | | of 400 mg/m2 of doxorubicin or doxorubicin equivalents |
| capecitabine | Xeloda | Initial therapy of patients with metastatic colorectal carcinoma when |
| | | treatment with fluoropyrimidine therapy alone is preferred. |
| | | Combination chemotherapy has shown a survival benefit compared |
| | | to 5-FU/LV alone. A survival benefit over 5_FU/LV has not been |
| | | demonstrated with Xeloda monotherapy. |
| capecitabine | Xeloda | Treatment in combination with docetaxel of patients with metastatic |
| | | breast cancer after failure of prior anthracycline containing |
| | | chemotherapy |
| carboplatin | Paraplatin | Palliative treatment of patients with ovarian carcinoma recurrent |
| | | after prior chemotherapy, including patients who have been |
| | | previously treated with cisplatin. |
| carboplatin | Paraplatin | Initial chemotherapy of advanced ovarian carcinoma in combination |
| | | with other approved chemotherapeutic agents. |
| carmustine | BCNU, BiCNU | |
| carmustine with | Gliadel Wafer | For use in addition to surgery to prolong survival in patients with |
| Polifeprosan 20 | | recurrent glioblastoma multiforme who qualify for surgery. |
| Implant | | |
| celecoxib | Celebrex | Accel. Approv. (clinical benefit not established) Reduction of polyp |
| | | number in patients with the rare genetic disorder of familial |
| | | adenomatous polyposis. |
| chlorambucil | Leukeran | Chronic Lymphocytic Leukemia- palliative therapy |
| cisplatin | Platinol | Metastatic testicular-in established combination therapy with other |
| | | approved chemotherapeutic agents in patients with metastatic |
| | | testicular tumors who have already received appropriate surgical |
| | | and/or radiotherapeutic procedures. An established combination |
| | | therapy consists of Platinol, Blenoxane and Velbam. |
| cisplatin | Platinol | Metastatic ovarian tumors - in established combination therapy with |
| | | other approved chemotherapeutic agents: Ovarian-in established |
| | | combination therapy with other approved chemotherapeutic agents |
| | | in patients with metastatic ovarian tumors who have already |
| | | received appropriate surgical and/or radiotherapeutic procedures. |
| | | An established combination consists of Platinol and Adriamycin. |
| | | Platinol, as a single agent, is indicated as secondary therapy in |
| | | patients with metastatic ovarian tumors refractory to standard |
| | | chemotherapy who have not previously received Platinol therapy. |
| cisplatin | Platinol | as a single agent for patients with transitional cell bladder cancer |
| | | which is no longer amenable to local treatments such as surgery |
| | | and/or radiotherapy. |
| cladribine | Leustatin, 2-CdA | Treatment of active hairy cell leukemia. |
| cyclophosphamide | Cytoxan, Neosar | |
| cyclophosphamide | Cytoxan Injection | |
| cyclophosphamide | Cytoxan Tablet | |
| cytarabine | Cytosar-U | |
| cytarabine liposomal | DepoCyt | Accel. Approv. (clinical benefit not established) Intrathecal therapy |
| | | of lymphomatous meningitis |
| dacarbazine | DTIC-Dome | |
| dactinomycin, | Cosmegan | |
| actinomycin D | | |
| Darbepoetin alfa | Aranesp | Treatment of anemia associated with chronic renal failure. |
| Darbepoetin alfa | Aranesp | Aranesp is indicated for the treatment of anemia in patients with |
| | | non- myeloid malignancies where anemia is due to the effect of |
| | | concomitantly administered chemotherapy. |
| daunorubicin | DanuoXome | First line cytotoxic therapy for advanced, HIV related Kaposi's |
| liposomal | | sarcoma. |
| daunorubicin, | Daunorubicin | Leukemia/myelogenous/monocytic/erythroid of adults/remission |
| daunomycin | | induction in acute lymphocytic leukemia of children and adults. |
| daunorubicin, | Cerubidine | In combination with approved anticancer drugs for induction of |
| daunomycin | | remission in adult ALL. |
| Denileukin diftitox | Ontak | Accel. Approv. (clinical benefit not established) treatment of patients |
| | | with persistent or recurrent cutaneous T-cell lymphoma whose |
| | | malignant cells express the CD25 component of the IL-2 receptor |
| dexrazoxane | Zinecard | Accel. Approv. (clinical benefit subsequently established) |
| | | Prevention of cardiomyopathy associated with doxorubicin |
| | | administration |
| dexrazoxane | Zinecard | reducing the incidence and severity of cardiomyopathy associated |
| | | with doxorubicin administration in women with metastatic breast |
| | | cancer who have received a cumulative doxorubicin dose of 300 |
| | | mg/m2 and who will continue to receive doxorubicin therapy to |
| | | maintain tumor control. It is not recommended for use with the |
| | | initiation of doxorubicin therapy. |
| docetaxel | Taxotere | Accel. Approv. (clinical benefit subsequently established) |
| | | Treatment of patients with locally advanced or metastatic breast |
| | | cancer who have progressed during anthracycline-based therapy or |
| | | have relapsed during anthracycline-based adjuvant therapy. |
| docetaxel | Taxotere | For the treatment of locally advanced or metastatic breast cancer |
| | | which has progressed during anthracycline-based treatment or |
| | | relapsed during anthracycline-based adjuvant therapy. |
| docetaxel | Taxotere | For locally advanced or metastatic non-small cell lung cancer after |
| | | failure of prior platinum-based chemotherapy. |
| docetaxel | Taxotere | in combination with cisplatin for the treatment of patients with |
| | | unresectable, locally advanced or metastatic non-small cell lung |
| | | cancer who have not previously received chemotherapy for this |
| | | condition. |
| doxorubicin | Adriamycin, Rubex | |
| doxorubicin | Adriamycin PFS | Antibiotic, antitumor agent. |
| | Injectionintravenous | |
| | injection | |
| doxorubicin liposomal | Doxil | Accel. Approv. (clinical benefit not established) Treatment of AIDS- |
| | | related Kaposi's sarcoma in patients with disease that has |
| | | progressed on prior combination chemotherapy or in patients who |
| | | are intolerant to such therapy. |
| doxorubicin liposomal | Doxil | Accel. Approv. (clinical benefit not established) Treatment of |
| | | metastatic carcinoma of the ovary in patient with disease that is |
| | | refractory to both paclitaxel and platinum based regimens |
| DROMOSTANOLONE | DROMOSTANOLONE | |
| PROPIONATE | | |
| DROMOSTANOLONE | MASTERONE | |
| PROPIONATE | INJECTION | |
| Elliott's B Solution | Elliott's B Solution | Diluent for the intrathecal administration of methotrexate sodium |
| | | and cytarabine for the prevention or treatment of meningeal |
| | | leukemia or lymphocytic lymphoma. |
| epirubicin | Ellence | A component of adjuvant therapy in patients with evidence of |
| | | axillary node tumor involvement following resection of primary |
| | | breast cancer. |
| Epoetin alfa | epogen | EPOGENB is indicated for the treatment of anemia related to |
| | | therapy with zidovudine in HIV- infected patients. EPOGENB is |
| | | indicated to elevate or maintain the red blood cell level (as |
| | | manifested by the hematocrit or hemoglobin determinations) and to |
| | | decrease the need for transfusions in these patients. EPOGEND is |
| | | not indicated for the treatment of anemia in HIV-infected patients |
| | | due to other factors such as iron or folate deficiencies, hemolysis or |
| | | gastrointestinal bleeding, which should be managed appropriately. |
| Epoetin alfa | epogen | EPOGENB is indicated for the treatment of anemic patients |
| | | (hemoglobin > 10 to _<13 g/dL) scheduled to undergo elective, |
| | | noncardiac, nonvascular surgery to reduce the need for allogeneic |
| | | blood transfusions. |
| Epoetin alfa | epogen | EPOGENB is indicated for the treatment of anemia in patients with |
| | | non-myeloid malignancies where anemia is due to the effect of |
| | | concomitantly administered chemotherapy. EPOGEND is indicated |
| | | to decrease the need for transfusions in patients who will be |
| | | receiving concomitant chemotherapy for a minimum of 2 months. |
| | | EPOGENB is not indicated for the treatment of anemia in cancer |
| | | patients due to other factors such as iron or folate deficiencies, |
| | | hemolysis or gastrointestinal bleeding, which should be managed |
| | | appropriately. |
| Epoetin alfa | epogen | EPOGEN is indicated for the treatment of anemia associated with |
| | | CRF, including patients on dialysis (ESRD) and patients not on |
| | | dialysis. |
| estramustine | Emcyt | palliation of prostate cancer |
| etoposide phosphate | Etopophos | Management of refractory testicular tumors, in combination with |
| | | other approved chemotherapeutic agents. |
| etoposide phosphate | Etopophos | Management of small cell lung cancer, first-line, in combination with |
| | | other approved chemotherapeutic agents. |
| etoposide phosphate | Etopophos | Management of refractory testicular tumors and small cell lung |
| | | cancer. |
| etoposide, VP-16 | Vepesid | Refractory testicular tumors-in combination therapy with other |
| | | approved chemotherapeutic agents in patients with refractory |
| | | testicular tumors who have already received appropriate surgical, |
| | | chemotherapeutic and radiotherapeutic therapy. |
| etoposide, VP-16 | VePesid | In combination with other approved chemotherapeutic agents as |
| | | first line treatment in patients with small cell lung cancer. |
| etoposide, VP-16 | Vepesid | In combination with other approved chemotherapeutic agents as |
| | | first line treatment in patients with small cell lung cancer. |
| exemestane | Aromasin | Treatment of advance breast cancer in postmenopausal women |
| | | whose disease has progressed following tamoxifen therapy. |
| Filgrastim | Neupogen | NEUPOGEN is indicated to reduce the duration of neutropenia and |
| | | neutropenia-related clinical sequelae, eg, febrile neutropenia, in |
| | | patients with nonmyeloid malignancies undergoing myeloablative |
| | | chemotherapy followed by marrow transplantation. |
| Filgrastim | Neupogen | NEUPOGEN is indicated to decrease the incidence of infection, as |
| | | manifested by febrile neutropenia, in patients with nonmyeloid |
| | | malignancies receiving myelosuppressive anticancer drugs |
| | | associated with a significant incidence of severe neutropenia with |
| | | fever. |
| Filgrastim | Neupogen | NEUPOGEN is indicated for reducing the time to neutrophil |
| | | recovery and the duration of fever, following induction or |
| | | consolidation hemotherapy treatment of adults with AML. |
| floxuridine | FUDR | |
| (intraarterial) | | |
| fludarabine | Fludara | Palliative treatment of patients with B-cell lymphocytic leukemia |
| | | (CLL) who have not responded or have progressed during |
| | | treatment with at least one standard alkylating agent containing |
| | | regimen. |
| fluorouracil, 5-FU | Adrucil | prolong survival in combination with leucovorin |
| fulvestrant | Faslodex | the treatment of hormone receptor-positive metastatic breast |
| | | cancer in postmenopausal women with disease progression |
| | | following antiestrogen therapy |
| gemcitabine | Gemzar | Treatment of patients with locally advanced (nonresectable stage II or |
| | | III) or metastatic (stage IV) adenocarcinoma of the pancreas. |
| | | indicated for first-line treatment and for patients previously treated |
| | | with a 5-fluorouracil-containing regimen. |
| gemcitabine | Gemzar | For use in combination with cisplatin for the first-line treatment of |
| | | patients with inoperable, locally advanced (Stage IIIA or IIIB) or |
| | | metastatic (Stage IV) non-small cell lung cancer. |
| gemtuzumab | Mylotarg | Accel. Approv. (clinical benefit not established) Treatment of CD33 |
| ozogamicin | | positive acute myeloid leukemia in patients in first relapse who are |
| | | 60 years of age or older and who are not considered candidates for |
| | | cytotoxic chemotherapy. |
| goserelin acetate | Zoladex Implant | Palliative treatment of advanced breast cancer in pre- and |
| | | perimenopausal women. |
| goserelin acetate | Zoladex | |
| hydroxyurea | Hydrea | Decrease need for transfusions in sickle cell anemia |
| Ibritumomab Tiuxetan | Zevalin | Accel. Approv. (clinical benefit not established) treatment of patients |
| | | with relapsed or refractory low-grade, follicular, or transformed B- |
| | | cell non-Hodgkin's lymphoma, including patients with Rituximab |
| | | refractory follicular non-Hodgkin's lymphoma. |
| idarubicin | Idamycin | For use in combination with other approved antileukemic drugs for |
| | | the treatment of acute myeloid leukemia (AML) in adults. |
| idarubicin | Idamycin | In combination with other approved antileukemic drugs for the |
| | | treatment of acute non-lymphocytic leukemia in adults. |
| ifosfamide | IFEX | Third line chemotherapy of germ cell testicular cancer when used in |
| | | combination with certain other approved antineoplastic agents. |
| imatinib mesylate | Gleevec | Accel. Approv. (clinical benefit not established) Initial therapy of |
| | | chronic myelogenous leukemia |
| imatinib mesylate | Gleevec | Accel. Approv. (clinical benefit not established) metastatic or |
| | | unresectable malignant gastrointestinal stromal tumors |
| imatinib mesylate | Gleevec | Accel. Approv. (clinical benefit not established) Initial treatment of |
| | | newly diagnosed Ph+ chronic myelogenous leukemia (CML). |
| Interferon alfa-2a | Roferon-A | |
| Interferon alfa-2b | Intron A | Interferon alfa-2b, recombinant for injection is indicated as adjuvant |
| | | to surgical treatment in patients 18 years of age or older with |
| | | malignant melanoma who are free of disease but at high risk for |
| | | systemic recurrence within 56 days of surgery. |
| Interferon alfa-2b | Intron A | Interferon alfa-2b, recombinant for Injection is indicated for the initial |
| | | treatment of clinically aggressive follicular Non-Hodgkin's |
| | | Lymphoma in conjunction with anthracycline-containing |
| | | combination chemotherapy in patients 18 years of age or older. |
| Interferon alfa-2b | Intron A | Interferon alfa-2b, recombinant for Injection is indicated for |
| | | intralesional treatment of selected patients 18 years of age or older |
| | | with condylomata acuminata involving external surfaces of the |
| | | genital and perianal areas. |
| Interferon alfa-2b | Intron A | Interferon alfa-2b, recombinant for Injection is indicated for the |
| | | treatment of chronic hepatitis C in patients 18 years of age or older |
| | | with compensated liver disease who have a history of blood on |
| | | blood-product exposure and/or are HCV antibody positive. |
| Interferon alfa-2b | Intron A | Interferon alfa-2b, recombinant for Injection is indicated for the |
| | | treatment of chronic hepatitis B in patients 18 years of age or older |
| | | with compensated liver disease and HBV replication. |
| Interferon alfa-2b | Intron A | Interferon alfa-2b, recombinant for Injection is indicated for the |
| | | treatment of patients 18 years of age or older with hairy cell |
| | | leukemia. |
| Interferon alfa-2b | Intron A | Interferon alfa-2b, recombinant for Injection is indicated for the |
| | | treatment of selected patients 18 years of age or older with AIDS- |
| | | Related Kaposi's Sarcoma. The likelihood of response to INTRON |
| | | A therapy is greater in patients who are without systemic symptoms, |
| | | who have limited lymphadenopathy and who have a relatively intact |
| | | immune system as indicated by total CD4 count. |
| irinotecan | Camptosar | Accel. Approv. (clinical benefit subsequently established) |
| | | Treatment of patients with metastatic carcinoma of the colon or |
| | | rectum whose disease has recurred or progressed following 5-FU- |
| | | based therapy. |
| irinotecan | Camptosar | Follow up of treatment of metastatic carcinoma of the colon or |
| | | rectum whose disease has recurred or progressed following 5-FU- |
| | | based therapy. |
| irinotecan | Camptosar | For first line treatment in combination with 5-FU/leucovorin of |
| | | metastatic carcinoma of the colon or rectum. |
| letrozole | Femara | Treatment of advanced breast cancer in postmenopausal women. |
| letrozole | Femara | First-line treatment of postmenopausal women with hormone |
| | | receptor positive or hormone receptor unknown locally advanced or |
| | | metastatic breast cancer. |
| letrozole | Femara | |
| leucovorin | Wellcovorin, | Leucovorin calcium is indicated for use in combination with 5- |
| | Leucovorin | fluorouracil to prolong survival in the palliative treatment of patients, |
| | | with advanced colorectal cancer. |
| leucovorin | Leucovorin | In combination with fluorouracil to prolong survival in the palliative |
| | | treatment of patients with advanced colorectal cancer. |
| levamisole | Ergamisol | Adjuvant treatment in combination with 5-fluorouracil after surgical |
| | | resection in patients with Dukes' Stage C colon cancer. |
| lomustine, CCNU | CeeBU | |
| meclorethamine, | Mustargen | |
| nitrogen mustard | | |
| megestrol acetate | Megace | |
| melphalan, L-PAM | Alkeran | Systemic administration for palliative treatment of patients with |
| | | multiple myeloma for whom oral therapy is not appropriate. |
| mercaptopurine, 6-MP | Purinethol | |
| mesna | Mesnex | Prevention of ifosfamide-induced hemorrhagic cystitis |
| methotrexate | Methotrexate | osteosarcoma |
| methoxsalen | Uvadex | For the use of UVADEX with the UVAR Photopheresis System in |
| | | the palliative treatment of the skin manifestations of cutaneous T- |
| | | cell lymphoma (CTCL) that is unresponsive to other forms of |
| | | treatment. |
| mitomycin C | Mutamycin | |
| mitomycin C | Mitozytrex | therapy of disseminated adenocarcinoma of the stomach or |
| | | pancreas in proven combinations with other approved |
| | | chemotherapeutic agents and as palliative treatment when other |
| | | modalities have failed. |
| mitotane | Lysodren | |
| mitoxantrone | Novantrone | For use in combination with corticosteroids as initial chemotherapy |
| | | for the treatment of patients with pain related to advanced hormone- |
| | | refractory prostate cancer. |
| mitoxantrone | Novantrone | For use with other approved drugs in the initial therapy for acute |
| | | nonlymphocytic leukemia (ANLL) in adults. |
| nandrolone | Durabolin-50 | |
| phenpropionate | | |
| Nofetumomab | Verluma | |
| Oprelvekin | Neumega | Neumega is indicated for the prevention of severe |
| | | thrombocytopenia and the reduction of the need for platelet |
| | | transfusions following myelosuppressive chemotherapy in adult |
| | | patients with nonmyeloid malignancies who are at high risk of |
| | | severe thrombocytopenia. |
| oxaliplatin | Eloxatin | Accel. Approv. (clinical benefit not established) in combination with |
| | | infusional 5-FU/LV, is indicated for the treatment of patients with |
| | | metastatic carcinoma of the colon or rectum whose disease has |
| | | recurred or progressed during or within 6 months of completion of |
| | | first line therapy with the combination of bolus 5-FU/LV and |
| | | irinotecan. |
| paclitaxel | Paxene | treatment of advanced AIDS-related Kaposi's sarcoma after failure |
| | | of first line or subsequent systemic chemotherapy |
| paclitaxel | Taxol | Treatment of patients with metastatic carcinoma of the ovary after |
| | | failure of first-line or subsequent chemotherapy. |
| paclitaxel | Taxol | Treatment of breast cancer after failure of combination |
| | | chemotherapy for metastatic disease or relapse within 6 months of |
| | | adjuvant chemotherapy. Prior therapy should have included an |
| | | anthracycline unless clinically contraindicated. |
| paclitaxel | Taxol | New dosing regimen for patients who have failed initial or |
| | | subsequent chemotherapy for metastatic carcinoma of the ovary |
| paclitaxel | Taxol | second line therapy for AIDS related Kaposi's sarcoma. |
| paclitaxel | Taxol | For first-line therapy for the treatment of advanced carcinoma of the |
| | | ovary in combination with cisplatin. |
| paclitaxel | Taxol | for use in combination with cisplatin, for the first-line treatment of |
| | | non-small cell lung cancer in patients who are not candidates for |
| | | potentially curative surgery and/or radiation therapy. |
| paclitaxel | Taxol | For the adjuvant treatment of node-positive breast cancer |
| | | administered sequentially to standard doxorubicin-containing |
| | | combination therapy. |
| paclitaxel | Taxol | First line ovarian cancer with 3 hour infusion. |
| pamidronate | Aredia | Treatment of osteolytic bone metastases of breast cancer in |
| | | conjunction with standard antineoplastic therapy. |
| pegademase | Adagen (Pegademase | Enzyme replacement therapy for patients with severe combined |
| | Bovine) | immunodeficiency as a result of adenosine deaminase deficiency. |
| Pegaspargase | Oncaspar | |
| Pegfilgrastim | Neulasta | Neulasta is indicated to decrease the incidence of infection, as |
| | | manifested by febrile neutropenia, in patients with non-myeloid |
| | | malignancies receiving myelosuppressive anti-cancer drugs |
| | | associated with a clinically significant incidence of febrile |
| | | neutropenia. |
| pentostatin | Nipent | Single agent treatment for adult patients with alpha interferon |
| | | refractory hairy cell leukemia. |
| pentostatin | Nipent | Single-agent treatment for untreated hairy cell leukemia patients |
| | | with active disease as defined by clinically significant anemia, |
| | | neutropenia, thrombocytopenia, or disease-related symptoms. |
| | | (Supplement for front -line therapy.) |
| pipobroman | Vercyte | |
| plicamycin, | Mithracin | |
| mithramycin | | |
| porfimer sodium | Photofrin | For use in photodynamic therapy (PDT) for palliation of patients with |
| | | completely obstructing esophageal cancer, or patients with partially |
| | | obstructing esophageal cancer who cannot be satisfactorily treated |
| | | with ND-YAG laser therapy. |
| porfimer sodium | Photofrin | For use in photodynamic therapy for treatment of microinvasive |
| | | endobronchial nonsmall cell lung cancer in patients for whom |
| | | surgery and radiotherapy are not indicated. |
| porfimer sodium | Photofrin | For use in photodynamic therapy (PDT) for reduction of obstruction |
| | | and palliation of symptoms in patients with completely or partially |
| | | obstructing endobroncial nonsmall cell lung cancer (NSCLC). |
| procarbazine | Matulane | |
| quinacrine | Atabrine | |
| Rasburicase | Elitek | ELITEK is indicated for the initial management of plasma uric acid |
| | | levels in pediatric patients with leukemia, lymphoma, and solid |
| | | tumor malignancies who are receiving anti-cancer therapy expected |
| | | to result in tumor lysis and subsequent elevation of plasma uric acid. |
| Rituximab | Rituxan | |
| Sargramostim | Prokine | |
| streptozocin | Zanosar | Antineoplastic agent. |
| talc | Sclerosol | For the prevention of the recurrence of malignant pleural effusion in |
| | | symptomatic patients. |
| tamoxifen | Nolvadex | As a single agent to delay breast cancer recurrence following total |
| | | mastectomy and axillary dissection in postmenopausal women with |
| | | breast cancer (T1-3, N1, M0) |
| tamoxifen | Nolvadex | For use in premenopausal women with metastatic breast cancer as |
| | | an alternative to oophorectomy or ovarian irradiation |
| tamoxifen | Nolvadex | For use in women with axillary node-negative breast cancer |
| | | adjuvant therapy. |
| tamoxifen | Nolvadex | Metastatic breast cancer in men. |
| tamoxifen | Nolvadex | Equal bioavailability of a 20 mg Nolvadex tablet taken once a day |
| | | to a 10 mg Nolvadex tablet taken twice a day. |
| tamoxifen | Nolvadex | to reduce the incidence of breast cancer in women at high risk for |
| | | breast cancer |
| tamoxifen | Nolvadex | In women with DCIS, following breast surgery and radiation, |
| | | Nolvadex is indicated to reduce the risk of invasive breast cancer. |
| temozolomide | Temodar | Accel. Approv. (clinical benefit not established) Treatment of adult |
| | | patients with refractory anaplastic astrocytoma, i.e., patients at first |
| | | relapse with disease progression on a nitrosourea and procarbazine |
| | | containing regimen |
| teniposide, VM-26 | Vumon | In combination with other approved anticancer agents for induction |
| | | therapy in patients with refractory childhood acute lymphoblastic |
| | | leukemia (all). |
| testolactone | Teslac | |
| thioguanine, 6-TG | Thioguanine | |
| thiotepa | Thioplex | |
| topotecan | Hycamtin | Treatment of patients with metastatic carcinoma of the ovary after |
| | | failure of initial or subsequent chemotherapy. |
| topotecan | Hycamtin | Treatment of small cell lung cancer sensitive disease after failure of |
| | | first-line chemotherapy. In clinical studies submitted to support |
| | | approval, sensitive disease was defined as disease responding to |
| | | chemotherapy but subsequently progressing at least 60 days (in the |
| | | phase 3 study) or at least 90 days (in the phase 2 studies) after |
| | | chemotherapy |
| toremifene | Fareston | Treatment of advanced breast cancer in postmenopausal women. |
| Tositumomab | Bexxar | Accel. Approv. (clinical benefit not established) Treatment of |
| | | patients with CD20 positive, follicular, non-Hodgkin's lymphoma, |
| | | with and without transformation, whose disease is refractory to |
| | | Rituximab and has relapsed following chemotherapy |
| Trastuzumab | Herceptin | HERCEPTIN as a single agent is indicated for the treatment of |
| | | patients with metastatic breast cancer whose tumors overexpress |
| | | the HER2 protein and who have received one or more |
| | | chemotherapy regimens for their metastatic disease. |
| Trastuzumab | Herceptin | Herceptin in combination with paclitaxel is indicated for treatment of |
| | | patients with metastatic breast cancer whose tumors overexpress |
| | | the HER-2 protein and had not received chemotherapy for their |
| | | metastatic disease |
| tretinoin, ATRA | Vesanoid | Induction of remission in patients with acute promyelocytic leukemia |
| | | (APL) who are refractory to or unable to tolerate anthracycline |
| | | based cytotoxic chemotherapeutic regimens. |
| Uracil Mustard | Uracil Mustard | |
| | Capsules | |
| valrubicin | Valstar | For intravesical therapy of BCG-refractory carcinoma in situ (CIS) |
| | | of the urinary bladder in patients for whom immediate cystectomy |
| | | would be associated with unacceptable morbidity or mortality. |
| vinblastine | Velban | |
| vincristine | Oncovin | |
| vinorelbine | Navelbine | Single agent or in combination with cisplatin for the first-line |
| | | treatment of ambulatory patients with unresectable, advanced non- |
| | | small cell lung cancer (NSCLC). |
| vinorelbine | Navelbine | Navelbine is indicated as a single agent or in combination with |
| | | cisplatin for the first-line treatment of ambulatory patients with |
| | | unreseactable, advanced non-small cell lung cancer (NSCLC). In |
| | | patients with Stage IV NSCLC, Navelbine is indicated as a single |
| | | agent or in combination with cisplatin. In Stage III NSCLC, |
| | | Navelbine is indicated in combination with cisplatin. |
| zoledronate | Zometa | the treatment of patients with multiple myeloma and patients with |
| | | documented bone metastases from solid tumors, in conjunction with |
| | | standard antineoplastic therapy. Prostate cancer should have |
| | | progressed after treatment with at least one hormonal therapy |

### Formulation and Administration:

The compounds of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to one or more compounds (according to any of the respective embodiments described herein) accountable for the biological effect.

Hereinafter, the phrases “physiologically acceptable carrier” and “pharmaceutically acceptable carrier”, which may be interchangeably used, refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

The term “tissue” refers to part of an organism consisting of cells designed to perform a function or functions. Examples include, but are not limited to, brain tissue, retina, skin tissue, hepatic tissue, pancreatic tissue, bone, cartilage, connective tissue, blood tissue, muscle tissue, cardiac tissue brain tissue, vascular tissue, renal tissue, pulmonary tissue, gonadal tissue, hematopoietic tissue.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose; and/or physiologically acceptable polymers such as polyvinyl pyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multi-dose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g., a compound according to any of the respective embodiments described herein, optionally in combination with an additional agent described herein) effective to prevent, alleviate or ameliorate symptoms of a disorder (e.g., a proliferative disease or disorder) or prolong the survival of the subject being treated.

Determination of a therapeutically effective amount is well within the capability of those skilled in the art, especially in light of the detailed disclosure provided herein. For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels (e.g., blood levels) of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed herein.

### Additional Definitions

Herein, the term “hydrocarbon” describes an organic moiety that includes, as its basic skeleton, a chain of carbon atoms, substituted mainly by hydrogen atoms. The hydrocarbon can be saturated or non-saturated, be comprised of aliphatic, alicyclic or aromatic moieties, and can optionally be substituted by one or more substituents (other than hydrogen). A substituted hydrocarbon may have one or more substituents, whereby each substituent group can independently be, for example, cycloalkyl, alkenyl, alkynyl, aryl, heteroaryl, heteroalicyclic, amine, halide, sulfate, sulfonate, sulfonyl, sulfoxide, phosphate, phosphonyl, phosphinyl, hydroxy, alkoxy, aryloxy, thiohydroxy, thioalkoxy, thioaryloxy, oxo, cyano, nitro, azo, azide, sulfonamide, carbonyl, thiocarbonyl, carboxy, thiocarbamate, urea, thiourea, carbamate, amide, epoxide and hydrazine. The hydrocarbon can be an end group or a linking group, as these terms are defined herein. Preferably, the hydrocarbon moiety has 1 to 20 carbon atoms.

As used herein throughout, the term “alkyl” refers to any saturated aliphatic hydrocarbon including straight chain and branched chain groups. Preferably, the alkyl group has 1 to 20 carbon atoms.

Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 1 to 10 carbon atoms. Most preferably, unless otherwise indicated, the alkyl is a lower alkyl having 1 to 4 carbon atoms. The alkyl group may be substituted or non-substituted.

When substituted, the substituent group can be, for example, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

Herein, the term “alkenyl” describes an unsaturated aliphatic hydrocarbon comprise at least one carbon-carbon double bond, including straight chain and branched chain groups. Preferably, the alkenyl group has 2 to 20 carbon atoms. More preferably, the alkenyl is a medium size alkenyl having 2 to 10 carbon atoms. Most preferably, unless otherwise indicated, the alkenyl is a lower alkenyl having 2 to 4 carbon atoms. The alkenyl group may be substituted or non-substituted.

Substituted alkenyl may have one or more substituents, whereby each substituent group can independently be, for example, alkynyl, cycloalkyl, alkynyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

Herein, the term “alkynyl” describes an unsaturated aliphatic hydrocarbon comprise at least one carbon-carbon triple bond, including straight chain and branched chain groups. Preferably, the alkynyl group has 2 to 20 carbon atoms. More preferably, the alkynyl is a medium size alkynyl having 2 to 10 carbon atoms. Most preferably, unless otherwise indicated, the alkynyl is a lower alkynyl having 2 to 4 carbon atoms. The alkynyl group may be substituted or non-substituted.

Substituted alkynyl may have one or more substituents, whereby each substituent group can independently be, for example, cycloalkyl, alkenyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

The term “alkylene” describes a saturated or unsaturated aliphatic hydrocarbon linking group, as this term is defined herein, which differs from an alkyl group (when saturated) or an alkenyl or alkynyl group (when unsaturated), as defined herein, only in that alkylene is a linking group rather than an end group.

A “cycloalkyl” group refers to a saturated on unsaturated all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, cyclohexadiene, cycloheptane, cycloheptatriene, and adamantane. A cycloalkyl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. When a cycloalkyl group is unsaturated, it may comprise at least one carbon-carbon double bond and/or at least one carbon-carbon triple bond. The cycloalkyl group can be an end group, as this phrase is defined herein, wherein it is attached to a single adjacent atom, or a linking group, as this phrase is defined herein, connecting two or more moieties.

An “aryl” group refers to an all-carbon monocyclic or fused-ring polycyclic (i.e., rings which share adjacent pairs of carbon atoms) end groups having a completely conjugated pi-electron system. Examples, without limitation, of aryl groups are phenyl, naphthalenyl and anthracenyl. The aryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. The aryl group can be an end group, as this phrase is defined herein, wherein it is attached to a single adjacent atom, or a linking group, as this phrase is defined herein, connecting two or more moieties.

A “heteroaryl” group refers to a monocyclic or fused ring (i.e., rings which share an adjacent pair of atoms) end group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or non-substituted. When substituted, the substituent group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein.

The term “arylene” describes a monocyclic or fused-ring polycyclic linking group, as this term is defined herein, and encompasses linking groups which differ from an aryl or heteroaryl group, as these groups are defined herein, only in that arylene is a linking group rather than an end group.

A “heteroalicyclic” group refers to a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or non-substituted. When substituted, the substituted group can be, for example, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, oxo, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, as these terms are defined herein. Representative examples are piperidine, piperazine, tetrahydrofuran, tetrahydropyran, morpholine and the like. The heteroalicyclic group can be an end group, as this phrase is defined herein, wherein it is attached to a single adjacent atom, or a linking group, as this phrase is defined herein, connecting two or more moieties.

Herein, the terms “amine” and “amino” each refer to either a —NR′R″ end group, a —N⁺R′R″R′″ end group, a —NR′— linking group, or a —N⁺R′R″— linking group, wherein R′, R″ and R′″ are each hydrogen or a substituted or non-substituted alkyl, alkenyl, alkynyl, cycloalkyl, heteroalicyclic (linked to amine nitrogen via a ring carbon thereof), aryl, or heteroaryl (linked to amine nitrogen via a ring carbon thereof), as defined herein. Optionally, R′, R″ and R′″ are hydrogen or alkyl comprising 1 to 4 carbon atoms. Optionally, R′ and R″ (and R′″, if present) are hydrogen. When substituted, the carbon atom of an R′, R″ or R′″ hydrocarbon moiety which is bound to the nitrogen atom of the amine is preferably not substituted by oxo, such that R′, R″ and R′″ are not (for example) carbonyl, C-carboxy or amide, as these groups are defined herein, unless indicated otherwise.

An “azide” group refers to a —N═N⁺═N⁻ group.

An “alkoxy” group refers to both an —O-alkyl and an —O-cycloalkyl end group, as defined herein, or to an —O-alkylene- or —O-cycloalkyl- linking group, as defined herein.

An “aryloxy” group refers to both an —O-aryl and an —O-heteroaryl end group, as defined herein, or to an —O-arylene- linking group, as defined herein.

A “hydroxy” group refers to a —OH group.

A “thiohydroxy” or “thiol” group refers to a —SH group.

A “thioalkoxy” group refers to both an —S-alkyl end group and an —S-cycloalkyl end group, as defined herein, or to an —S-alkylene- or —S-cycloalkyl- linking group, as defined herein.

A “thioaryloxy” group refers to both an —S-aryl and an —S-heteroaryl end group, as defined herein, or to an —S-arylene- linking group, as defined herein.

A “carbonyl” group refers to a —C(═O)—R′ end group, where R′ is defined as hereinabove, or to a —C(═O)— linking group.

A “thiocarbonyl” group refers to a —C(═S)—R′ end group, where R′ is as defined herein, or to a —C(═S)— linking group.

A “carboxyl”, “carboxylic” or “carboxylate” refers to both “C-carboxy” and O-carboxy” end groups, as well as to a —C(═O)—O—linking group.

A “C-carboxy” group refers to a —C(═O)—O—R′ group, where R′ is as defined herein.

An “O-carboxy” group refers to an R′C(═O)—O—group, where R′ is as defined herein.

A “carboxylic acid” refers to a —C(═O)OH group, including the deprotonated ionic form and salts thereof.

An “ester” refers to a —C(═O)OR′ group, wherein R′ is not hydrogen.

An “oxo” group refers to a ═O group.

A “thiocarboxy” or “thiocarboxylate” group refers to both —C(═S)—O—R′ and —O—C(═S)R′ end groups, or to a —C(═S)—O—linking group.

A “halo” group refers to fluorine, chlorine, bromine or iodine.

A “haloalkyl” group refers to an alkyl group substituted by one or more halo groups, as defined herein.

A “sulfinyl” group refers to an —S(═O)—R′ end group, where R′ is as defined herein, or to a —S(═O)— linking group.

A “sulfonyl” group refers to an —S(═O)₂—R′ end group, where R′ is as defined herein, or to a —S(═O)₂— linking group.

A “sulfonate” group refers to an —S(═O)₂—O—R′ end group, where R′ is as defined herein, or to a S(═O)₂—O— linking group.

A “sulfate” group refers to an —O—S(═O)₂—O—R′ end group, where R′ is as defined as herein, or to a —O—S(═O)₂—O— linking group.

A “sulfonamide” or “sulfonamido” group encompasses both S-sulfonamido and N-sulfonamido end groups, as defined herein, and a —S(═O)₂—NR′— linking group.

An “S-sulfonamido” group refers to a —S(═O)₂—NR′R″ group, with each of R′ and R″ as defined herein.

An “N-sulfonamido” group refers to an R'S(═O)₂—NR″ group, where each of R′ and R″ is as defined herein.

A “carbamyl” or “carbamate” group encompasses O-carbamyl and N-carbamyl end groups, and to a —OC(═O)—NR′—linking group.

An “O-carbamyl” group refers to an —OC(═O)—NR′R″ group, where each of R′ and R″ is as defined herein.

An “N-carbamyl” group refers to an R′OC(═O)—NR″—group, where each of R′ and R″ is as defined herein.

A “thiocarbamyl” or “thiocarbamate” group encompasses O-thiocarbamyl and N-thiocarbamyl end groups, and to a —OC(═S)—NR′—linking group.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

An “amide” or “amido” group encompasses C-amido and N-amido end groups, as defined herein, and to a —C(═O)—NR′—linking group.

A “C-amido” group refers to a —C(═O)—NR′R″ group, where each of R′ and R″ is as defined herein.

An “N-amido” group refers to an R′C(═O)—NR″—group, where each of R′ and R″ is as defined herein.

A “urea group” refers to an —N(R′)—C(═O)—NR″R′″ end group, or to a —N(R′)—C(═O)—NR″—linking group, where each of R′, R″ and R″ is as defined herein.

A “thiourea group” refers to a —N(R′)—C(═S)—NR″R′″ end group, or to a —N(R′)—C(═S)—NR″—linking group where each of R′, R″ and R″ is as defined herein.

A “nitro” group refers to an —NO₂ group.

A “cyano” group refers to a —C≡N group.

The term “phosphonyl” or “phosphonate” describes a —P(═O)(OR′)(OR″) end group, or a —P(═O)(OR′)—O— linking group, with R′ and R″ as defined hereinabove.

The term “phosphate” describes an —O—P(═O)(OR′)(OR″) end group, or a —O—P(═O)(OR′)—O— linking group with each of R′ and R″ as defined hereinabove.

The term “phosphinyl” describes a —PR′R″ end group, or —PRR′— linking group, with each of R′ and R″ as defined hereinabove.

The term “hydrazine” describes a —NR′—NR″R′″ end group, or —NR′—NR″— linking group, with R′, R″, and R′″ as defined herein.

As used herein, the term “hydrazide” describes a —C(═O)—NR′—NR″R′″ end group, or —C(═O)—NR′—NR″— linking group, where R′, R″ and R′″ are as defined herein.

As used herein, the term “thiohydrazide” describes a —C(═S)—NR′—NR″R′″ end group, or —C(═S)—NR′—NR″— linking group, where R′, R″ and R′″ are as defined herein.

A “guanidinyl” group refers to an —RaNC(═NRd)-NRbRc end group, or —RaNC(═NRd)-NRb-linking group where each of Ra, Rb, Rc and Rd can be as defined herein for R′ and R″.

A “guanyl” or “guanine” group refers to an RaRbNC(═NRd)-end group, or a —RaNC(═NRd)-linking group, where Ra, Rb and Rd are as defined herein.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Methods

Materials:

All solvents and reagents used for organic synthesis were obtained from Sigma-Aldrich, Merck, Baker and/or Acros and used without further purification.

Building blocks for synthesis were obtained from Enamine and MolPort.

Purification of precursors was performed using an automated Flash chromatography system (CombiFlash® Systems, Teledyne Isco, USA) with RediSep® Rf Normal-phase Flash Columns. Final compounds were purified by semi-preparative HPLC on a Waters Prep 2545 Preparative Chromatography System, with UV/Vis detector 2489, using XBridge® Prep C1810 μm 10×250 mm Column (PN: 186003891, SN:16113608512502). LC-MS-ESI spectra of products and reaction progress were monitored using a Waters UPLC®-MS system: Acquity™ UPLC® H class with PDA detector, Acquity™ UPLC® BEH C181.7 μm 2.1×50 mm Column (PN:186002350, SN 02703533825836), Waters SQ detector 2.

Electrophile Library Screening:

993 compounds were transferred to a 384-well plate working copy by combining 0.5 μl of 20 mM stock solution of four or five compounds per well. The catalytic domain of Pin1 (2 μM) in 20 mM Tris, 75 mM NaCl, pH 7.5 was incubated with 200 μM for each compound and moderately shaken for 24 hours at 4° C. The reaction was stopped by the addition of formic acid to 0.4% (v/v) final concentration.

Liquid chromatography/mass spectroscopy runs were performed on an Acquity™ UPLC® H-class system (Waters) in positive ion mode using electrospray ionization (ESI). UPLC separation was performed on a C4 column (300 Å, 1.7 μM, 21 mm×100 mm). The column was held at 40° C., and the autosampler at 10° C. Mobile solution A was 0.1% formic acid in water and mobile phase B was 0.1% formic acid in acetonitrile. Run flow was 0.4 ml/minute; and a gradient of 20% B for 2 minutes, increasing linearly to 60% B for 3 minutes, holding at 60% B for 1.5 minutes, changing to 0% B in 0.1 minute and holding at 0% for 1.4 minutes, was used. Desolvation temperature was 500° C. with a flow rate of 1000 liters/hour. The capillary voltage was 0.69 kV and cone voltage 46 V. Raw data was processed using OpenLynx™ software and deconvoluted using the MaxEnt tool. Labeling assignment was performed as described in Resnick et al. [J Am Chem Soc 2019, 141:8951-8968].

Covalent Docking:

Covalent docking was performed using DOCKovalent 3.7 [London et al., Nat Chem Biol 2014, 10:1066-1072] against 16 structures of Pin1. PDB codes: 1PIN, 2ITK, 2Q5A, 2XP3, 2ZQV, 2ZR4, 3IK8, 3KAB, 3KCE, 3NTP, 3ODK, 3OOB, 3TC5, 3TCZ, 3TDB, 3WHO. The docked compounds included seven sulfolane hits from the electrophilic library with the following IDs: PCM-0102138, PCM-0102178, PCM-0102105, PCM-0102832, PCM-0102313, PCM-0102760, PCM-0102755. The covalent bond length was set to 1.8 Å and the two newly formed bond angles to Cβ-Sγ-C=109.5±5° and Sγ-C-Ligatom=109.5±5°.

### Preparation of 448 Triazole Analog Library for In Situ Mass Spectroscopic (MS) Screening

Click reactions were conducted on a 0.2 μmol scale in 384-well plates (Greiner). In each well, azide in DMSO (28.57 mM, 8.75 μl, 1.25 equivalent), Pin1-4 in DMSO (100 mM, 2 μl, 1 equivalent), tert-butanol (10.15 μl), aqueous sodium ascorbate solution (1.5 mM, 26.7 μl, 0.2 equivalent), 1:1 CuSO₄/THBTA (tris(3-hydroxypropyltriazolylmethyl)amine) in 1:1 DMSO/H₂O (2.5 mM, 2.4 μl, 0.03 equivalent) were dispensed using a multi-channel pipette. Each well contained 50 μl reaction mixture with a final product concentration of 4 mM, provided complete reaction. The plate was sealed and incubated overnight on a shaker at room temperature. A working plate was prepared by diluting the products in DMSO to reach a final concentration of 50 μM.

In Situ Mass Spectroscopic (MS) Screening of Triazole Analog Library:

For the screening 2 μl of each of the 448 click products as 50 μM DMSO stocks were transferred into a 384-well plate working plate. 48 μl of catalytic domain of Pin1 (2 μM) in 20 mM Tris (pH 7.5) with 75 mM NaCl were added and moderately shaken and incubated for 15 minutes at room temperature. The reaction was stopped by the addition of formic acid to 0.4% (v/v) final concentration (20 μl). The mixtures were analyzed by liquid chromatography/mass spectroscopy analogously to the electrophile library incubations described herein. Hits were retrospectively analyzed by liquid chromatography/mass spectroscopy (LC-MS) to ensure reaction completion.

Labeling Assignment and Processing of Mass Spectrometry Data:

For each measured well, processed peaks were searched to match the mass of unlabeled protein or common small adducts of the unlabeled protein, which were found in the control sample or labeled protein. Labeling percentage for a compound was determined as the labeling of a specific compound divided by the overall detected protein species. Peaks whose mass could not be assigned were discarded from the overall labeling calculation. Data was analyzed using a python script for processing the MaxEnt-deconvoluted spectra. Peaks were normalized from ion counts to percentages, where the highest peak is defined as 100%. The unlabeled protein mass is deduced from a reference well that contains just the protein.

Thiol Reactivity Assay:

50 μM DTNB (dithionitrobenzoic acid) was incubated with 200 μM TCEP (tris(2-carboxytehyl)phosphine) in 20 mM sodium phosphate buffer (pH 7.4) with 150 mM NaCl, for 5 minutes at room temperature, in order to obtain TNB²⁻ (thionitrobenzoate dianion). 200 μM compounds were subsequently added to the TNB²⁻, followed by immediate UV absorbance measurement at 412 nm (at 37° C.). The UV absorbance was acquired every 15 minutes for 7 hours. The assay was performed in a 384-well plate using a Spark™ 10M plate reader (Tecan). Background absorbance of compounds was subtracted by measuring the absorbance at 412 nm of each compound under the same conditions without DTNB. Compounds were measured in triplicates. The data was fitted to a second order reaction equation such that the rate constant k is the slope of ln([A][B₀]/[B][A₀]), where [A₀] and [B₀] are the initial concentrations of the compound (200 μM) and TNB²⁻ (100 μM) respectively, and [A] and [B] are the remaining concentrations as a function of time, as determined from the spectrometric measurement. Linear regression using Prism® software was performed to fit the rate against the first four hours of measurements.

Cell Viability Assay:

MDA-MB-231 cells grew in DMEM medium supplemented with 10% FCS (fetal calf serum), 1% PS (penicillin-streptomycin) and 1% L-glutamine (all from Biological Industries). Exclusion of mycoplasma contamination was monitored and conducted by test with MycoAlert™ kit (Lonza). Cells were trypsinized and counted, and 1000 cells/well were plated in 50 μl of growth medium into 384-well white TC plates (Greiner) using Multidrop™ 384 (Thermo Scientific) Washer Dispenser II. The number of viable cells was monitored using CellTiter-Glo® Luminescent kit (Promega) in accordance with the manufacturer's protocol. Luminescence was measured using luminescence module of PHERAstar™ FS plate reader (BMG Labtech). Data analysis was performed using GeneData 12 analytic software. Assay ready plate preparation: Compounds transferred into black microplates (Greiner 784900) using Labcyte Echo® acoustic dispensing technology. Assay ready plates were then sealed with heat seals. If not used immediately, plates were frozen at −20° C. and held in polypropylene boxes with silica-gel desiccant.

Fluorescence Polarization (FP) Assay:

Binding affinity to Pin1 was determined using a fluorescence polarization assay to assess competition with an N-terminal fluorescein-labeled peptide (Bth-D-phosThr-Pip-Nal), which was obtained from JPT Peptide Technologies and Proteintech Group. The indicated concentrations of candidate compound were pre-incubated for 12 hours at 4° C. with a solution containing 250 nM glutathione S-transferase (GST)-Pin1, 5 nM of fluorescein-labeled peptide probe, 10 μg/ml bovine serum albumin, 0.01% Tween-20 and 1 mM DTT (dithiothreitol) in a buffer of 10 mM HEPES, 10 mM NaCl and 1% glycerol (pH 7.4). Measurements of FP were performed in black 384-well plates (Corning) using an EnVision™ reader. Apparent Kᵢ values (under the tested conditions) obtained from the FP assay results were derived from the Kenakin Kᵢ equation:

Kenakin Kᵢ=(Lb)(EC₅₀)(K_{d})/(Lo)(Ro)+Lb(Ro−Lo+Lb−K_{d})

wherein K_{d} [M]: K_{d} of the probe, EC₅₀ [M]: obtained from FP assay, total tracer Lo [M]: probe concentration in FP, bound tracer Lb [M]: 85% of probe concentration binds to target protein, total receptor Ro [M]: Pin1 concentration in the FP assay, as described [Auld D. S. et al., Receptor binding assays for HTS and drug discovery. in Assay Guidance Manual eds. Sittampalam G. S. et al., Eli Lilly & Company and the National Center for Advancing Translational Sciences, 2004].

Pin1 Substrate Activity Assay:

Inhibition of Pin1 isomerase activity was determined using the chymotrypsin-coupled PPIase assay, using GST-Pin1 and Suc-Ala-pSer-Pro-Phe-pNA (SEQ ID NO: 2) peptide substrate (50 mM), according to procedures described by Yaffe [Science 1997, 278:1957-1960]. GST-Pin1 was pre-incubated with the indicated concentrations of compound for 12 hours at 4° C. in buffer containing 35 mM HEPES (pH 7.8), 0.2 mM DTT, and 0.1 mg/ml BSA (bovine serum albumin). Immediately before the assay was started, chymotrypsin (final concentration of 6 mg/ml), followed by the peptide substrate (Suc-Ala-pSer-Pro-Phe-pNA (SEQ ID NO: 2) peptide substrate, final concentration 50 mM) was added. The apparent Kᵢ value (under the tested conditions) obtained from the PPIase assay was derived from the Cheng-Prusoff equation:

Kᵢ=IC₅₀/(1+S/Kₘ)

wherein Kₘ is the Michaelis constant for the used substrate, S is the initial concentration of the substrate in the assay, and IC₅₀ is the half-minimal inhibitory concentration of the inhibitor.

Immunoblotting:

Whole cell lysates for immunoblotting were prepared by pelleting cells from each cell line at 4° C. (at 300 g) for 5 minutes. The resulting cell pellets were washed 1× with ice-cold 1×PBS and then re-suspended in the indicated cell lysis buffer. Lysates were clarified at 14,000 rotations per minute for 15 minutes at 4° C. prior to quantification using a BCA assay kit (Pierce, cat. #23225). Whole cell lysates were loaded into Bolt™ 4-12% Bis-Tris Gels (Thermo Fisher, cat. #NW04120BOX) and separated by electrophoreses at 95 V for 1.5 hour. The gels were transferred to a nitrocellulose membrane using the iBlot® Gel Transfer device (Thermo Fisher, cat. #IB23001) at P3 for 6 minutes and then blocked for 1 hour at room temperature in Odyssey® blocking buffer (LI-COR Biosciences, cat. #927-50010). Membranes were probed using antibodies against the relevant proteins at 4° C. overnight in 20% Odyssey® blocking buffer in 1×TBST (Tris buffered saline with Tween™ 20). Membranes were then washed three times with 1×TBST (at least 5 minutes per wash) followed by incubation with the IRDye® goat anti-mouse (LI-COR Biosciences, cat. #926-32210) or goat anti-rabbit (LI-COR Biosciences, cat. #926-32211) secondary antibody (diluted 1:10,000) in 20% Odyssey® blocking buffer in 1×TBST for 1 hour at room temperature. After three washes with 1×TBST (at least 5 minutes per wash), the immunoblots were visualized using the Odyssey® Infrared Imaging System (LI-COR Biosciences).

Lysate Pull-Down Assays:

The indicated cells were treated with increasing concentrations of either DMSO, Pin1-3, or Pin1-3-AcA for 5 hours. Cells were harvested by scraping and washed twice with PBS before lysis with 50 mM HEPES (pH 7.4), 1 mM EDTA, 10% glycerol, 1 mM TCEP, 150 mM NaCl, 1 mM EDTA, 0.5% NP-40, and protease inhibitor tablet (Roche cat. #4693159001). After clarifying (14,000 rpm for 15 minutes), samples were treated with the indicated concentrations of Pin1-3-DTB at 4° C. for 1 hour. Lysates were then incubated with streptavidin agarose resin (Thermo Scientific, cat. #20349) for 1.5 hour at 4° C. Beads were washed four times with 500 μl of washing buffer (50 mM HEPES (pH 7.5), 10 mM NaCl, 1 mM EDTA, 10% glycerol), then pelleted by centrifugation and dried. The beads were boiled for 5 minutes at 95° C. in 2×LDS+10% β-mercaptoethanol. Proteins of interest were then assessed via Western blotting using the bolt system (Life Technologies).

Cellular Target Engagement—Live Cell Competition Assays:

The indicated cells were plated in 10 cm plates with 2.5 million cells per plate in 6 ml of medium. The day after plating, cells were treated with the indicated concentrations of candidate inhibitor for the indicated time points. The cells were then washed two times with cold phosphate buffer saline (1 ml per 10 cm plate) and collected by scraping with a cell scraper. Cells were lysed in 50 mM HEPES (pH 7.4), 1 mM EDTA, 10% glycerol, 1 mM TCEP, 150 mM NaCl, 1 mM EDTA, 0.5% NP-40, and protease inhibitor tablet (Roche)-using 210 μl of cell lysis buffer per 10 cm plate of cells. After clarifying (14,000 rpm for 15 minutes), 9 μl of each lysate sample was combined with 5 μl of 4×LDS+10% β-mercaptoethanol (in a ratio of 3:1), boiled for 5 minutes, and set aside for the input loading control. Then, 200 μl of each lysate sample was incubated with 1 μM of Pin1-3-DTB for 1 hour at 4° C. and processed as described hereinabove for the lysate pull-down assays.

RNA Sequencing:

Mino cells (acquired from the ATCC) were grown at 37° C. in a 5% CO₂ humidified incubator and cultured in RPMI-1640 (Biological Industries), supplemented with 15% fetal bovine serum (Biological Industries) and 1% pen-strep solution (Biological Industries). 11×10⁶ cells were incubated with 1 μM Pin1-3 (0.02% DMSO) or with 0.02% DMSO in triplicates for 6 hours. Total RNA was isolated with RNeasy™ kit (Qiagen). RNA libraries were prepared from 2 μg total RNA using SENSE™ mRNA-Seq library prep kit V2 (Lexogen). Total RNA and library quality was analyzed using Qubit™ fluorometric and TapeStation™ analysis (Agilent). Samples were sequenced using NextSeg™ 500/550 High Output Kit v2.5 (Illumina) on NextSeg™ 550.

RNA-seq reads were aligned to the human genome (hg19 assembly) using STAR [Dobin et al., Bioinformatics 2013, 29:15-21] and gene expression was determined using RSEM [L₁ & Dewey, BMC Bioinformatics 2011, 12:323] and RefSeq annotations. Differential expression was computed using DESeq2 [Love et al., Genome Biol 2014, 15:550] with default parameters. Genes with baseMean >50 that were downregulated with P<0.05 were further analyzed using Enrichr [Kuleshov et al., Nucleic Acids Res 2016, 44:W90-W97].

Profiling of Pin1-3 Reactive Cysteines by rdTOP-ABPP:

MDA-MB-231 cells were cultured at 37° C. under a 5% CO₂ atmosphere in DMEM culture medium supplemented with 10% FBS and 1% PS. Cells were grown to 70% confluence and incubated with DMSO or 5 μM Pin1-3 for 2 hours with serum-free medium. Cells were harvested, lysed by sonication in ice-cold PBS containing 0.1% Triton™ X-100 and centrifuged at 100,000 g for 30 minutes to remove cell debris. Then protein concentrations were determined by BCA protein assay. Proteomes were normalized to 2 mg/ml in 1 ml for each sample. Each of the DMSO and Pin1-3 incubated proteomes was treated with 100 μM iodoacetamide alkyne for 1 hour at room temperature. The proteomes were then reacted with 1 mM CuSO₄, 100 μM TBTA (tris((1-benzyl-4-triazolyl)methyl)amine) ligand, 100 μM biotin-acid-N₃ tag and 1 mM TCEP (tris(2-carboxyethyl)phosphine) for 1 hour. After a click reaction, the proteomes were centrifuged at 8000 g for 5 minutes and then the precipitated proteins were washed for two times using cold methanol. The proteomes were re-suspended in 1.2% SDS/PBS and diluted to 0.2% SDS/PBS. Finally, the samples were prepared, analyzed on LC-MS/MS and quantified according to procedures described in Yang et al. [Anal Chem 2018, 90:9576-9582]. Briefly, the beads from trypsin digestion were washed and re-suspended in 100 μl of TEAB buffer. 8 μl of 4% D¹³CDO or HCHO was added to the Pin1-3 or DMSO sample respectively. At the same time, 8 μl of 0.6 M NaBH₃CN was added and the reaction was lasted for 2 hours at room temperature. The beads were then washed again and the modified peptides were cleaved by 2% formic acid. LC-MS/MS data was analyzed by ProLuCID™ algorithm (as described by Xu et al. [J Proteomics 2015, 129:16-24]) with static modification of cysteine (+57.0215 Da) and variable oxidation of methionine (+15.9949 Da). The isotopic modifications (+28.0313 and +34.0631 Da for light and heavy labeling respectively) are set as static modifications on the N-terminal of a peptide and lysines. Variable modification on cysteines is set at +322.23688 Da. The ratios were quantified by CImage™ software [Weerapana et al., Nature 2010, 468, 790-795].

Zebrafish Model of Neuroblastoma:

Zebrafish were used for a model of childhood neuroblastoma, in which the tissue-specific overexpression of the human MYCN transgene using the dopamine β hydroxylase (dβh) promoter in the zebrafish peripheral sympathetic nerve system (PSNS) drives neuroblastoma tumorigenesis in zebrafish [Zhu et al., Cancer Cell 2012, 21:362-373]. The fish are also transgenic for a PSNS-specific dβh:EGFP reporter line, so that the tumors can be visualized by EGFP. In this model, hyperproliferation of sympathetic neuroblasts is evident in the intrarenal gland (counterpart of the adrenal medulla) starting at 4 days post-fertilization (dpf).

Zebrafish embryos at 3 dpf were treated with different concentrations of the test compound in the egg water (reverse osmosis or RO water with 0.6 gm/liter instant ocean salts) for 4 days. The embryos were transferred to egg water containing freshly diluted drug after 2 days (5 dpf). The embryos were then imaged at 7 dpf, and the relative EGFP+MYCN-overexpressing neuroblast cross-sectional area for each experimental group was quantified.

Pin1 Expression and Purification:

A construct of full-length human Pin1 in a pET28 vector was overexpressed in E. coli BL21 (DE3) in LB medium in the presence of 50 mg/ml of kanamycin. Cells were grown at 37° C. to an optical density (OD) of 0.8, cooled to 17° C., induced with 500 μM isopropyl-1-thio-D-galactopyranoside, incubated overnight at 17° C., collected by centrifugation, and stored at −80° C. Cell pellets were sonicated in buffer A (50 mM HEPES, pH 7.5, 500 mM NaCl, 10% glycerol, 20 mM Imidazole, and 7 mM BME) and the resulting lysate was centrifuged at 30,000×g for 40 minutes. Ni-NTA beads (Qiagen) were mixed with lysate supernatant for 30 min and washed with buffer A. Beads were transferred to an FPLC-compatible column and the bound protein was washed with 15% buffer B (50 mM HEPES, pH 7.5, 500 mM NaCl, 10% glycerol, 250 mM imidazole, and 3 mM BME) and eluted with 100% buffer B. Thrombin was added to the eluted protein and incubated at 4° C. overnight. The sample was concentrated and passed through a Superdex™ 20010/300 column (GE Healthcare) in a buffer containing 20 mM HEPES, pH 7.5, 150 mM NaCl, 5% glycerol, and 1 mM TCEP. Fractions were pooled, concentrated to approximately 37 mg/ml and frozen at −80° C.

Pin1 Crystallization and Soaking:

Apo protein at a final concentration of 1 mM was crystallized by sitting-drop (200 nL+200 nL) vapor diffusion at 20° C. in the following crystallization buffer: 3 M NH₄SO₄, 100 mM HEPES, pH 7.5, 150 mM NaCl, 1% PEG400, and 10 mM DTT. A volume of 200 nL of 1 mM Pin1-3 was added directly to crystals for soaking at 20° C. for 16 hours. Crystals were transferred briefly into crystallization buffer containing 25% glycerol prior to flash-freezing in liquid nitrogen.

Crystallization Data Collection and Structure Determination:

Diffraction data from complex crystals were collected at beamline 24ID-C of the NE-CAT at the Advanced Photon Source at the Argonne National Laboratory. Data sets were integrated and scaled using XDS, as described by Kabsch [Acta Crystallogr D Biol Crystallogr 2010, 66:125-132]. Structures were solved by molecular replacement using the Phaser™ program, as described by McCoy et al. [J Appl Crystallogr 2007, 40:658-674], and the search model PDB entry 1PIN. Iterative manual model building and refinement using Phenix [Acta Crystallogr D Biol Crystallogr 2010, 66:213-221] and Coot [Emsley & Cowtan, Acta Crystallogr D Biol Crystallogr 2004, 60:2126-2132] led to models with excellent statistics.

Crystallization conditions and data collection and refinement statistics for crystal structures were as follows:

RCSB accession code: 6VAJ

Data collection (a single crystal was used to collect data for each reported structure):

Space group—P 4₃ 2₁ 2

Cell dimensions—a, b, c (Å) 48.9648.96137.04
a, b, g (°) 90.0090.0090.00

Resolution (Å)—39.84-1.42 (1.471-1.42) (Values in parentheses are for highest-resolution shell)

Rₚᵢₙ—0.01849 (0.5658)

Redundancy—6.2 (6.3)

Completeness (%)—99.38 (99.72)

I/σI—17.67 (1.54)

Structure Solution:

PDB entries used for molecular replacement—1PIN

Refinement:

No. reflections—32262 (3163)

R_{work}—0.1923 (0.3278)

R_{free}—0.2144 (0.3227)

No. atoms—1384
Macromolecules—1229
Ligand/ion—23
Water—132

B-factors—31.41
Macromolecules—30.11
Ligand/ion—50.67
Water—40.23

R.m.s. deviations
Bond lengths (Å)—0.006
Bond angles (°)—1.19

Ramachandran:

Preferred—100.0%

Allowed—0.0%

Not allowed 0.0%

NMR Spectroscopy:

Spectral analysis by ¹H- and ¹³C-NMR was obtained on a Bruker Avance™ 300 MHz and 400 MHz spectrometer, equipped with a QNP probe. Chemical shifts (δ_{H} & δ_{C}) are quoted in ppm to the nearest 0.1 ppm, and referenced to trimethylsilane (TMS). Coupling constants (J) are reported in Hertz (Hz) to the nearest 0.1 Hz.

### Example 1

### Identification of Pin1-Binding Compounds by Covalent Fragment Screening

A library of 993 electrophilic fragments containing 752 chloroacetamides and 241 acrylamides, as described in Resnick et al. [J Am Chem Soc 2019, 141:8951-8968], was screened against Pin1 in order to identify electrophilic scaffolds suitable for developing potent and selective Pin1 inhibitors. The electrophilic fragments serve as mildly reactive “warheads” capable of irreversibly binding cysteines in target proteins.

The purified catalytic domain of Pin1 was incubated with the fragment library (2 μM protein, 200 μM compound; 24 hours at 4° C.), followed by intact protein liquid chromatography/mass-spectrometry (LC/MS) to identify and quantify compound labeling. FIG. 1 depicts an example of a compound identified in this manner.

As shown in FIG. 2, 111 fragments irreversibly labeled Pin1 under the assay conditions by >50% (an 11.2% hit rate).

As shown in FIG. 2, FIG. 3 and Table 1 below, the 48 most potent hits (labeling >75%) included 9 chloroacetamides that shared a common cyclic sulfone moiety, indicative of a structure activity relationship (SAR).

As the identified sulfone-containing hits were non-promiscuous in previous fragment screens against a diverse panel of ten proteins [Resnick et al., J Am Chem Soc 2019, 141:8951-8968], these compounds were selected for further study. In order to avoid undesired reactivity arising from the presence of an additional Michael acceptor in the 2-sulfolene fragments, sulfolane analogs were used exclusively at this stage.

TABLE 1
Pin1-binding compounds uncovered by screening which comprise
a cyclic sulfone moiety (structures depicted in FIG. 3) - labeling
percentage determined via intact protein LC/MS after incubation
of 2 μM Pin1 with 200 μM test compound for 24 hours at 4° C.

| Compound | Labeling [%] |
|---|---|
| PCM-0102372 | 100 |
| PCM-0102760 | 100 |
| PCM-0102539 | 100 |
| PCM-0102579 | 100 |
| PCM-0102868 | 100 |
| PCM-0102230 | 87 |
| PCM-0102105 | 85 |
| PCM-0102755 | 83 |
| PCM-0102313 | 83 |
| PCM-0102178 | 72 |
| PCM-0102832 | 72 |
| PCM-0103082 | 69 |
| PCM-0102138 | 56 |
| PCM-0102896 | 42 |

### Example 2

### Selective Pin1-Binding Compounds

DOCKovalent [London et al., Nat Chem Biol 2014, 10:1066-1072] was used to generate docking predictions in order to visualize possible binding modes to Cys113 in the active site of Pin1. All sulfolane hits identified according to Example 1 were docked into various Pin1 structures and highly ranked poses were inspected.

As shown in FIG. 4, two plausible binding modes were predicted by docking of exemplary compounds to Pin1. In both poses, either the sulfolane moiety or the lipophilic moiety (R in formulas of FIG. 2): (i) protruded into the hydrophobic proline-binding pocket that is mainly formed by Met130, Gln131 and Phe134, or (ii) interacted with a hydrophobic patch adjacent to Cys113, formed by Ser115, Leu122 and Met130.

These results suggested that non-covalent binding affinity can be optimized by diversification of the lipophilic residue.

Based on the docking predictions, a total of 26 compounds that featured a range of small or bulky aliphatic, arylic, biphenylic or heterocyclic side-chains (structures depicted in FIG. 5), were synthesized or purchased. In order to identify potent binders, the irreversible labeling efficiency of these second-generation compounds was assessed alongside the original screening hits under more stringent conditions, with a 1:1 ratio of protein to compound (2 μM compound; 1 hour at room temperature).

As shown in Table 2, 25 of the 26 tested second-generation compounds exhibited better labeling than the original hits, which exhibited no labeling under these new conditions. The cyclohexyl residue-bearing Pin1-2-3 displayed the highest degree of labeling (65%). In addition, a wide range of lipophilic moieties were tolerated.

TABLE 2
Exemplary Pin1-binding compounds (structures depicted in FIGS. 3
and 5) - labeling percentage determined via intact protein LC/MS
after incubation of 2 μM Pin1 with 2 μM test compound for 1 hour
at room temperature

| | Labeling | Reactivity k | Reactivity |
|---|---|---|---|
| Compound | [%] | [M⁻¹ * second⁻¹] | Log k |
| Pin1-18 | n.d. | 1.69E−08 | −7.77 |
| Pin1-2-3 | 65 | 1.53E−07 | −6.82 |
| Pin1-2-8 | 52 | 2.19E−07 | −6.66 |
| Pin1-2-1 | 50 | 1.09E−07 | −6.96 |
| Pin1-3 | 48 | 3.73E−08 | −7.43 |
| Pin1-3-13 | 46 | 1.50E−07 | −6.82 |
| Pin1-3-9 | 46 | 3.42E−07 | −6.47 |
| Pin1-433 | 45 | 2.13E−07 | −6.67 |
| Pin1-2-9 | 43 | 7.68E−08 | −7.11 |
| Pin1-2-7 | 37 | 1.02E−07 | −6.99 |
| Pin1-3-7 | 36 | 1.12E−07 | −6.95 |
| Pin1-2-6 | 30 | 1.58E−07 | −6.80 |
| Pin1-053 | 28 | 1.24E−07 | −6.91 |
| Pin1-2-2 | 27 | 8.06E−08 | −7.09 |
| Pin1-3-14 | 27 | 7.03E−08 | −7.15 |
| Pin1-437 | 27 | 1.51E−07 | −6.82 |
| Pin1-128 | 25 | 1.47E−07 | −6.83 |
| Pin1-2-10 | 25 | 1.30E−07 | −6.89 |
| Pin1-2-5 | 24 | 1.31E−07 | −6.88 |
| Pin1-3-8 | 23 | 8.22E−08 | −7.09 |
| Pin1-3-15 | 21 | 7.77E−08 | −7.11 |
| Pin1-2-11 | 19 | 1.15E−07 | −6.94 |
| Pin1-838 | 16 | 1.41E−07 | −6.85 |
| Pin1-028 | 16 | 1.59E−07 | −6.80 |
| Pin1-324 | 12 | 1.59E−07 | −6.80 |
| Pin1-707 | 0 | 1.17E−09 | −8.93 |
| PCM-0102138 | 0 | 1.20E−07 | −6.92 |
| PCM-0102178 | 0 | 1.30E−07 | −6.89 |
| PCM-0102105 | 0 | 1.10E−07 | −6.96 |
| PCM-0102832 | 0 | 6.02E−08 | −7.22 |
| PCM-0102313 | 0 | 1.07E−07 | −6.97 |
| PCM-0102760 | 0 | 1.00E−07 | −7.00 |
| PCM-0102755 | 0 | 1.54E−07 | −6.81 |
| PCM-0102230 | 0 | 8.87E−08 | −7.05 |

As shown in FIG. 7 and Table 2, the compounds PCM-0102832, PCM-0102313, PCM-0102760 and PCM-0102755 correspond to Pin1-3-13, Pin1-3-14, Pin1-2-3 and Pin1-437, respectively, without a methylene group adjacent to the nitrogen of the amide group; and exhibited no labeling under the tested conditions, whereas Pin1-3-13, Pin1-3-14, Pin1-2-3 and Pin1-437 each exhibited significant labeling under such conditions.

These results indicate that an additional methylene group between the amide and the lipophilic side-chain was strongly associated with increased labeling efficiency, as four matched molecular pairs lacking this group exhibited no labeling.

TABLE 3
Exemplary Pin1-binding compounds (structures depicted in FIGS. 5
and 8) - labeling percentage determined via intact protein LC/MS
after incubation of 2 μM Pin1 with 2 μM test compound for 15
minutes at room temperature

| | Labeling | Reactivity k | Reactivity |
|---|---|---|---|
| Compound | [%] | [M⁻¹ * second⁻¹] | Log k |
| P1-01-B11 | 89 | 1.37E−07 | −6.86 |
| P1-03-G07 | 73 | 1.37E−06 | −5.86 |
| P1-02-H08 | 73 | 1.32E−06 | −5.88 |
| P1-03-C04 | 72 | 3.78E−07 | −6.42 |
| P1-02-E11 | 70 | 1.04E−06 | −5.98 |
| P1-04-B02 | 69 | 1.73E−06 | −5.76 |
| P1-01-G10 | 67 | 1.20E−07 | −6.92 |
| P1-01-F08 | 64 | 1.32E−06 | −5.88 |
| P1-02-B04 | 62 | 1.26E−06 | −5.90 |
| P1-03-D08 | 54 | 1.20E−06 | −5.92 |
| P1-01-B05 | 51 | 1.51E−06 | −5.82 |
| P1-02-B12 | 47 | 1.34E−06 | −5.87 |
| P1-03-A12 | 44 | 1.48E−06 | −5.83 |
| Pin1-2-3 | 42 | 1.53E−07 | −6.82 |
| P1-01-F11 | 39 | 6.81E−07 | −6.17 |
| P1-03-B04 | 34 | 1.66E−06 | −5.78 |
| Pin1-3 | 10 | 3.73E−08 | −7.43 |

For further optimization of the lipophilic moiety, an alkyne side chain-bearing analog was prepared, which was derivatized with 448 different azides using copper-catalyzed azide-alkyne cycloaddition (CuAAC). This library of 448 analogs was tested in the MS-labeling assay under stringent assay conditions (2 μM compound for 15 minutes at room temperature) to filter for high affinity binders.

37 of the tested compounds labeled Pin1 significantly faster than second generation binders. The structures of the 10 most potent Pin1-binding compounds from among the 37 tested compounds are depicted in FIG. 8.

As shown in Table 3, P1-01-B11 was the fastest binding compound, labeling 89% of Pin1 in 15 minutes.

In order to estimate the influence of the various lipophilic moieties on “warhead” reactivity [Flanagan et al., J Med Chem 2014, 57:10072-10079; Lonsdale et al., J Chem Inf Model 2017, 57:3124-3137; Dahal et al., Medchemcomm 2016, 7:864-872], the thiol reactivity of the top ten binders of the second and third generation was assessed using a high-throughput assay previously applied to the entire fragment library, as described in Resnick et al. [J Am Chem Soc 2019, 141:8951-8968]. In brief, the second-order rate constant was evaluated for a model thiol, which reflects trends in general reactivity towards thiol groups.

As shown in FIG. 9, there was no correlation between labeling efficiency and reactivity (Pearson R=0.003). This was particularly evident when comparing Pin1-3, which features a tert-butyl residue, and the structurally similar cyclopropyl residue-bearing Pin1-3-13. Furthermore, the compound with the highest degree of binding, Pin1-2-3, exhibited only median reactivity relative to the other compounds.

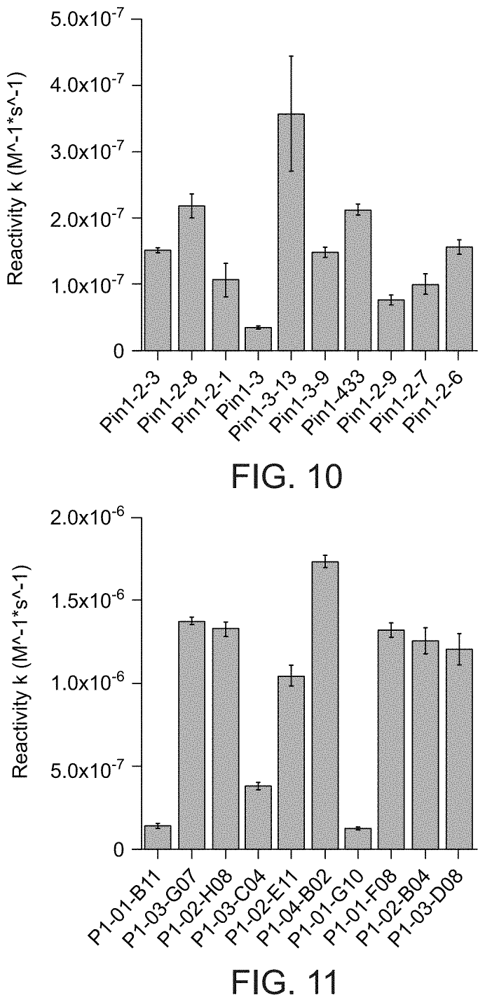

Similarly, as shown in FIG. 10, both Pin1-3 and Pin1-3-13 labeled Pin1 to essentially the same extent (48% and 46%), but their general reactivity varied by an order of magnitude.

Similarly, as shown in FIG. 11, the reactivities of the top ten third generation binders also vary significantly.

These results indicate that the binding of identified compounds represents specific interactions with Pin1, rather than non-specific reactivity.

### Example 3

### Non-Cytotoxic Pin1 Inhibition

Covalent labeling of Pin1 was confirmed to translate into enzyme inhibition via a fluorescence polarization (FP) competition assay using a FITC-labeled substrate mimetic peptide inhibitor, as well as a chymotrypsin-coupled PPIase assay, using procedures described in Wei et al. [Nat Med 2015, 21:457-466].

As shown in FIG. 12, FIG. 13 and Table 4, the compounds Pin1-3 and Pin1-3-13 showed comparable inhibition of Pin1 (substrate assay: 103 nM; fluorescence polarization assay: 110 nM vs. 121 nM).

As further shown in FIG. 13, all tested Pin1-binding compounds competed in the FP assay at least about as well as juglone, a known Pin1 inhibitor.

TABLE 4
Exemplary Pin1-binding compounds (structures depicted in FIG. 3) and their labeling percentage
(as determined by LC/MS), apparent Ki (as determined by FP assay), IC₅₀, EC₅₀ (as determined by
cell viability assay with MDA-MB-231 cells), and reactivity (as determined by DTNB assay)-
Pin1-3-AcA and juglone serve as non-reactive and reactive controls, respectively

| | | Ki | | | | |
|---|---|---|---|---|---|---|
| | Labeling | (apparent) | IC₅₀ | Reactivity k | | EC₅₀ |
| Compound | [%] | [nM] | [nM] | [M⁻¹*second⁻¹] | Log k | [μM] |
| Pin1-2-3 | 65 | 46 | n.d. | 1.53E−07 | −6.82 | 7.5 |
| Pin1-2-8 | 52 | 133 | n.d. | 2.19E−07 | −6.66 | 5.1 |
| Pin1-2-1 | 50 | 58 | n.d. | 1.09E−07 | −6.96 | 2.8 |
| Pin1-3 | 48 | 110 | 103 | 3.73E−08 | −7.43 | >25 |
| Pin1-3-13 | 46 | 121 | n.d. | 1.50E−07 | −6.82 | n.d. |
| Pin1-3-9 | 46 | 411 | n.d. | 3.42E−07 | −6.47 | n.d. |
| Pin1-433 | 45 | 40/194 | n.d. | 2.13E−07 | −6.67 | 8.9 |
| Pin1-2-9 | 43 | 83 | n.d. | 7.68E−08 | −7.11 | 11.3 |
| Pin1-2-7 | 37 | 39 | n.d. | 1.02E−07 | −6.99 | 6.1 |
| Pin1-2-6 | 30 | 194 | n.d. | 1.58E−07 | −6.80 | 5.6 |
| Pin1-3-AcA | n.d. | >100000 | n.d. | n.d. | n.d. | n.d. |
| Juglone | n.d. | 1750 | n.d. | n.d. | n.d. | n.d. |

The fluorescent polarization assay was performed in a dose-dependent and time-dependent manner, in order to further characterize the kinetic parameters of Pin1-3 binding to Pin1.

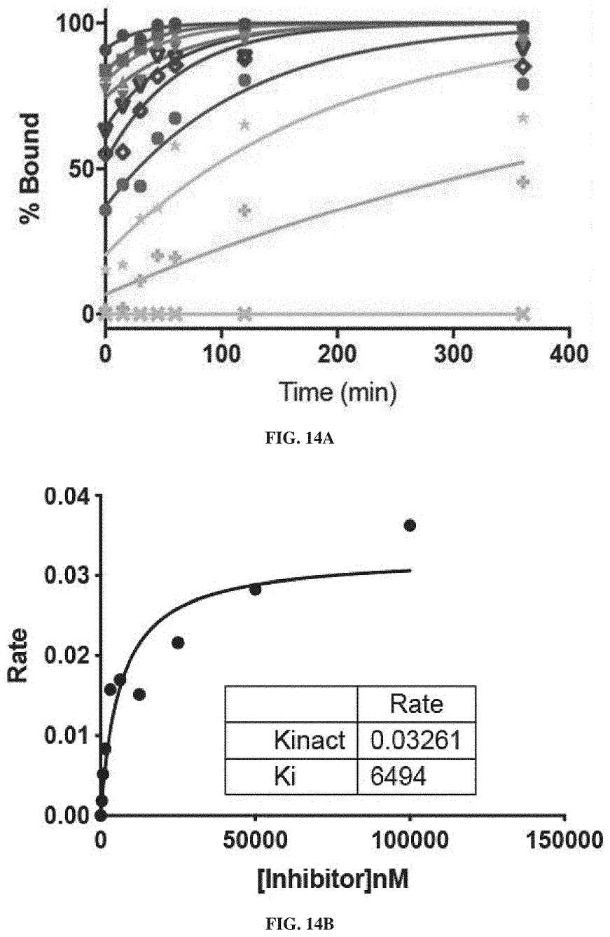

As shown in FIGS. 14A and 14B, the K_{inact} of Pin1-3 was determined by fluorescent polarization assay to be 0.03 minute⁻¹ and the ratio K_{inact}/Kᵢ (apparent) was an impressive 29,000 M⁻¹ second⁻¹.

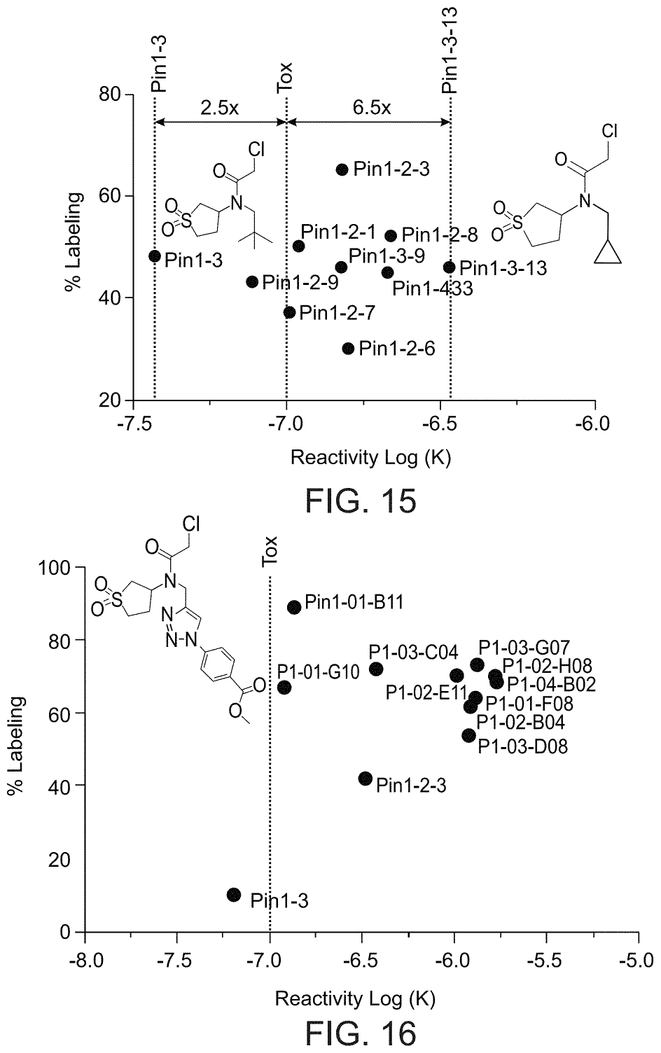

As shown in FIG. 15, Pin1-3 exhibits a combination of labeling efficiency and low reactivity.

Similarly, as shown in FIG. 16, P1-01-B11 also exhibits a combination of labeling efficiency and low reactivity.

These suggest indicate that Pin1-3 (second generation) and P1-01-B11 (third generation) would be particularly less likely to result in off-target activity. Pin1-3 and P1-01-B11 were therefore selected as a lead inhibitor, as previous studies suggest that high warhead reactivity can lead to nonspecific binding, resulting in off-target cytotoxicity [Ward et al., J Med Chem 2013, 56:7025-7048; Planken et al., J Med Chem 2017, 60:3002-3019; Cheng et al., J Med Chem 2016, 59:2005-2024].

Exemplary Pin1-binding compounds were also tested for non-selective cytotoxicity in a viability assay against IMR90 lung fibroblasts.

As further shown in Table 4, the cell viability assay confirmed that Pin1-3 was the least toxic compound with EC₅₀ values above 25 whereas other tested compounds exhibited cytotoxic effects with EC₅₀ values ranging from 2.8 μM to 11.3

These data suggest that Pin1-3 has the lowest inherent reactivity of the tested top Pin1-binding compounds, and does not exhibit non-selective cytotoxicity, therefore showing a particularly good balance of potency and selectivity.

### Example 4

### Crystal Structure of Pin1 with Exemplary Pin1-Binding Compound

The co-crystal structure of Pin1 in complex with Pin1-3 at 1.4 Å resolution was determined, in order to confirm Cys113 as the covalent target of Pin1-3 and gain insights into its binding mode.

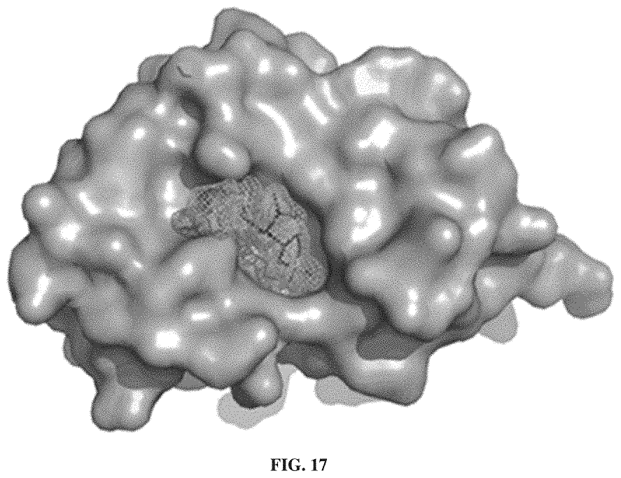

As shown in FIG. 17, Pin1-3 bound to the active site formed a covalent bond with the catalytic Cys113, which was clearly visible as continuous electron density in the 2F_{O}-F_{C} omit map.

As shown in FIGS. 18 and 19, the sulfolane ring occupies the hydrophobic Pro-binding pocket that is formed by Met130, Gln131, Phe134, Thr152 and His157, and the sulfonyl oxygens mediate hydrogen bonds with the backbone amide of Q131 and the imidazole NH of His157.

As shown in FIG. 19, the abovementioned hydrogen bonds are analogous to those featured in the binding of arsenic trioxide to Pin1, as described by Kozono et al. [Nat Commun 2018, 9:3069].

As further shown in FIGS. 18 and 19, the tert-butyl group of Pin1-3 covers a hydrophobic patch formed by Ser115, Leu122 and Met130. This shallow hydrophobic interface leaves the tert-butyl group mostly solvent-exposed and explains the broad range of hydrophobic moieties that were accepted at this position during the optimization efforts.

Overall, the above results indicate that Pin1-3, despite being a small ligand (heavy atom count: 17, c Log P: 0.36, LLE [Leeson & Springthorpe, Nat Rev Drug Discov 2007, 6:881-890]=7.34), efficiently exploits the active site of Pin1 even in the absence of a negatively charged moiety to interact with the phosphate binding pocket [Zhang et al., ACS Chem Biol 2007, 2:320-328]. Pin1-3 therefore overcomes the cell-permeability issues of previously developed Pin1 inhibitors, which are often highly anionic [Guo et al., Bioorganic Med Chem Lett 2009, 19:5613-5616; Dong et al., Bioorganic Med Chem Lett 2010, 20:2210-2214; Guo et al., Bioorganic Med Chem Lett 2014, 24:4187-4191].

### Example 5

### Selective Inhibition of Pin1 in Cells

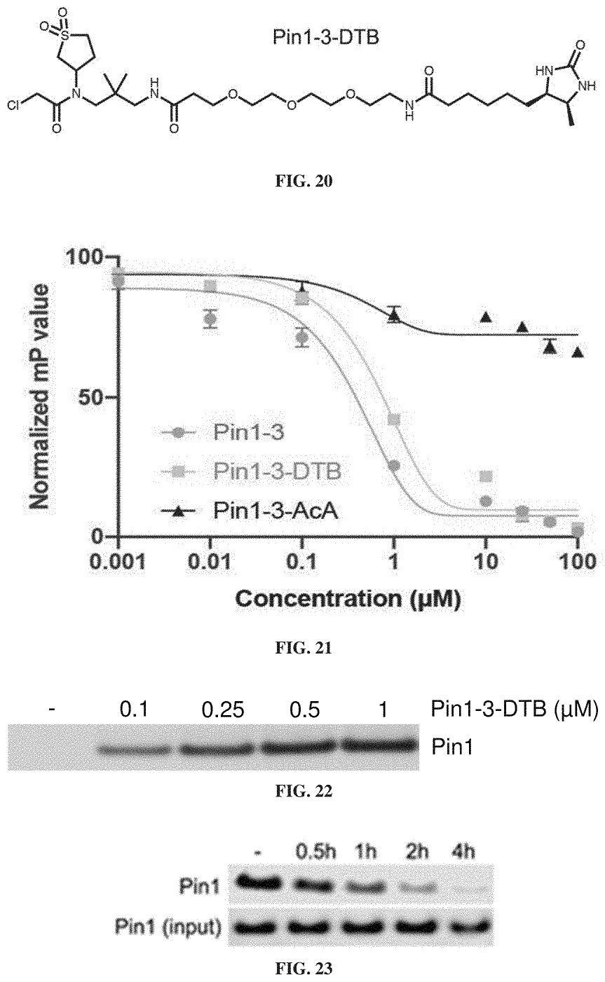

In order to assess the target engagement of Pin1-3 in cells, a desthiobiotin probe was developed for live-cell competition and pull-down experiments. Based on the co-crystal structure of Pin1-3 discussed in Example 4, the mostly solvent-exposed tert-butyl group was identified as the most suitable site for a PEG-linked desthiobiotin moiety in a labeled analog of Pin1-3, named Pin1-3-DTB (as depicted in FIG. 20). Importantly, this modification would not decrease the bulkiness of the tert-butyl moiety and hence the probe should retain a low reactivity profile.

As shown in FIG. 21, Pin1-3-DTB exhibited similar potency (apparent Ki=38 nM (under the tested conditions), as determined by fluorescence polarization assay) to that of Pin1-3.

In order to assess cell permeability of Pin1-3 as well as its ability to engage cellular Pin1, PATU-8988T cells were treated with Pin1-3 (0.25 to 1 μM) for 5 hours. After cell lysis, the lysates were incubated with Pin1-3-DTB (1 μM, 1 hour at 4° C.) and probe-labeled targets were pulled down with streptavidin beads.

As shown in FIG. 22, complete pull-down of 1 μM Pin1-3-DTB was observed after only 1 hour incubation.

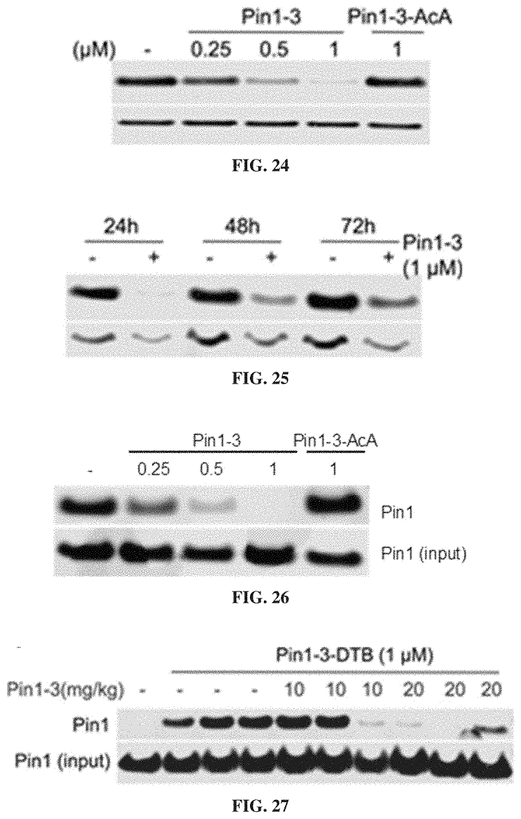

As shown in FIG. 24, Pin1-3 exhibited dose-dependent inhibition of Pin1-3-DTB pull-down, as determined by Western blotting of eluted proteins, with maximal competition observed at a concentration of 1 μM. In contrast, the negative control Pin1-3-AcA exhibited no competition.

As shown in FIG. 23, further incubations with a fixed Pin1-3 concentration (1 μM) but at varying incubations times (30 minutes to 4 hours) indicated that Pin1 binding occurs rapidly in cells (complete engagement within 4 hours, with about 50% engagement after 2 hours).

As shown in FIG. 25, the Pin1-3 maintained significant engagement to Pin1 for up to 72 hours in PATU-8988T cells.

As shown in FIG. 26, the Pin1-3 exhibited similar engagement to Pin1 in IMR32 cells.

Similar engagement of Pin1-3 to Pin1 was also observed in HCT116 and MDA-MB-231 cells (data not shown).

The in vivo engagement of Pin1 by Pin1-3 was then assessed using Pin1-3-DTB. Mice were treated with either vehicle, 10 mg/kg or 20 mg/kg Pin1-3 by oral gavage (QD) for 3 days, followed by lysis of the spleens for a competition pull-down experiment.

As shown in FIG. 27, effective Pin1 engagement by Pin1-3 was observed in 1 of the 3 mice treated with 10 mg/kg Pin1-3, and in all 3 mice treated with 20 mg/kg Pin1-3, with target engagement monitored by loss of Pin1-3-DTB-mediated pull-down.

Based on these results, a 40 mg/kg dose was chosen for further mice experiments to ensure complete Pin1 engagement.

These results indicate that Pin1-3 potently engages Pin1 in cells, both in vitro and in vivo.

In order to profile the selectivity of Pin1-3, a Covalent Inhibitor Target-site Identification (CITe-Id) experiment [Browne et al., J Chem Soc 2019, 141, 191-203] was performed, as depicted in FIG. 28.

This chemoproteomic platform enables the identification and quantification of the dose-dependent binding of covalent inhibitors to cysteine residues on a proteome-wide scale. In this competition experiment, live PATU-8988T cells were incubated with Pin1-3 (100, 500 or 1000 nM) for 5 hours, followed by cell lysis and co-incubation with Pin1-3-DTB (2 μM) for 18 hours. Following trypsin digest and avidin enrichment, the DTB-modified peptides were analyzed by shotgun LC-MS/MS.

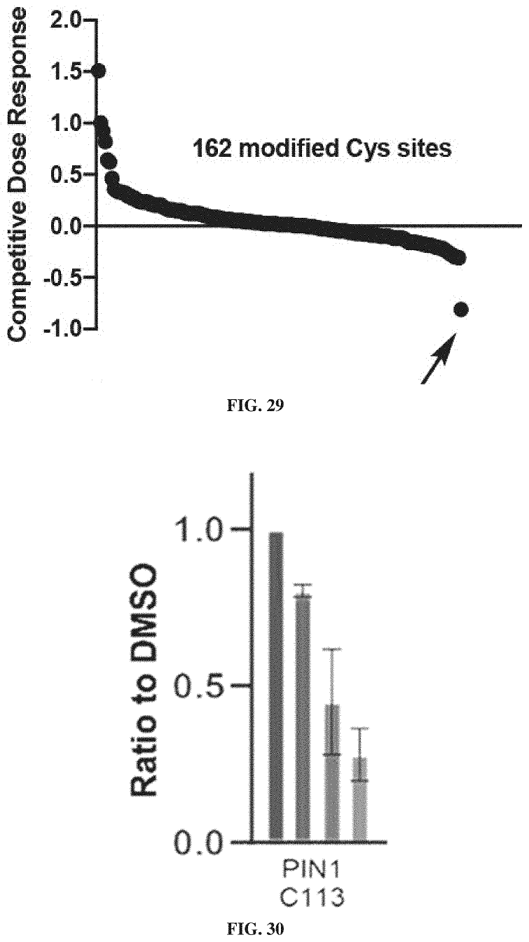

As shown in FIGS. 29 and 30, out of 162 cysteine residues labeled by Pin1-3-DTB in PATU-8988T cells, only Pin1 Cys113 exhibited dose-dependent competition (more than 2 standard deviations from the median) exhibited dose-dependent competition, indicating the pronounced selectivity of Pin1-3.

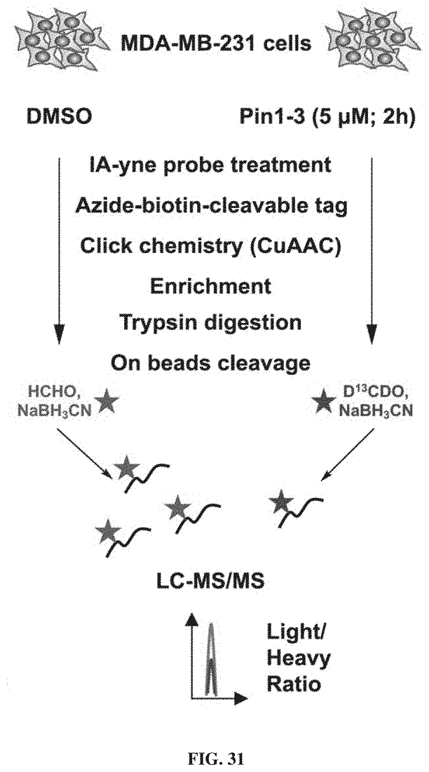

In order to further profile the selectivity of Pin1-3, an rdTOP-ABPP experiment was performed to profile its cysteine targets throughout the proteome, as depicted schematically in FIG. 31, using procedures described in Yang et al. [Anal Chem 2018, 90:9576-9582].

This variant of the isoTOP-ABPP technique enables the site-specific quantification of cysteine binding by label-free covalent inhibitors. In brief, MDA-MB-231 cells were treated with Pin1-3, lysed and labeled with a bioorthogonal iodoacetamide-alkyne probe that was then conjugated to a cleavable biotin tag by copper-catalyzed azide-alkyne cycloaddition (CuAAC). After enrichment on beads, the peptides were isotopically derivatized by triplex reductive dimethylation, cleaved and analyzed via LC-MS/MS analysis.

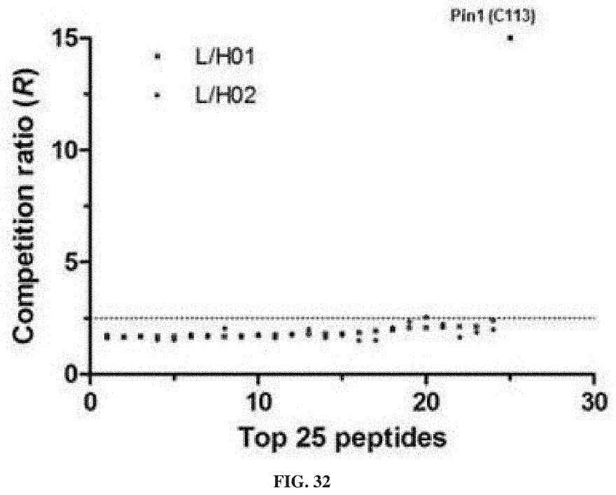

As shown in FIG. 32, Cys113 of Pin1 was identified as the top ranked cysteine labeled by Pin1-3 at a biologically relevant concentration (5 μM) in MDA-MB-231 cells, with a competition ratio R=15 across two biological replicates, whereas all other identified cysteines exhibited R values below 2.5. Out of 2134 identified cysteines in the experiment, only two cysteines showed light/heavy ration >2.5. Of these, one cysteine did not replicate, and only Pin1 C113 showed the maximal ratio of 15 in both replicates.

Taken together, the above results indicate that Pin1-3 has an exquisite selectivity profile, confirmed using independent chemoproteomic techniques in different cell lines, making it highly suitable for inhibition of Pin1 in cells and in vivo.

### Example 6

### Effect of Pin1-Binding Compound in Cancer Cells

In order to profile the anti-proliferative activity of Pin1-3, the compound was submitted to the PRISM platform (Broad Institute) to evaluate its potency against 300 suspension and hematopoietic human cancer cell lines. The PRISM method enables high-throughput, pooled screening of mixtures of cell lines, which are each labeled with a 24-nucleotide barcode [Yu et al., Nat Biotechnol 2016, 34:419-423]. In all 300 cancer cell lines profiled, Pin1-3 demonstrated limited to no anti-proliferative activity after a 5-day treatment, with IC₅₀ values >3 μM. This result aligns with the initial cytotoxicity screening, as well as data from the Cancer Dependency Map (Broad Institute), in which Pin1 was not identified as a significant genetic dependency in CRISPR-Cas9 and RNAi screens across hundreds of cancer cell lines (www[dot]depmap[dot]org/portal/). This suggests that the strong single-agent cytotoxicity of previously published Pin1 inhibitors, such as juglone, is likely attributable to off-targets.

The ability of Pin1-3 treatment to induce more pronounced anti-proliferative effects after prolonged treatment (6-8 days) was then assessed. In order to ensure that target engagement was maintained for the duration of the experiment, Pin1-3 was replenished in fresh media every 48 hours.

The effect of Pin1-binding compounds on 8988T pancreatic adenocarcinoma cells was assessed by incubating cells with 1 μM Pin1-3, and evaluating cell growth relative to cells incubated with vehicle (DMSO) alone. In order to confirm that the effect of Pin1-3 is mediated by Pin1, the experiment was repeated using Pin1 knockout cells.

As shown in FIG. 33, 1 μM of Pin1-3 reduced pancreatic cancer cell viability after 6-8 days in statistically significant manner.

As shown in FIG. 34, 1 μM Pin1-3 had no considerable effect on viability of Pin1 knockout cells (although on day 8, the small difference was statistically significant (p<0.01)), indicating that the inhibitory effect of Pin1-3 is mediated primarily by Pin1 modulation.

FIG. 35 confirms that the Pin1 knockout cells indeed lacked Pin1 expression.

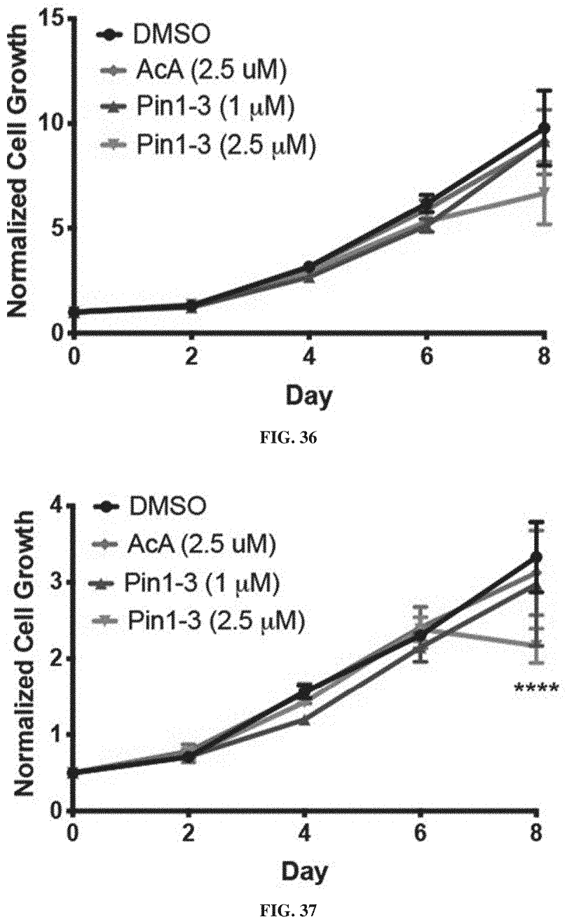

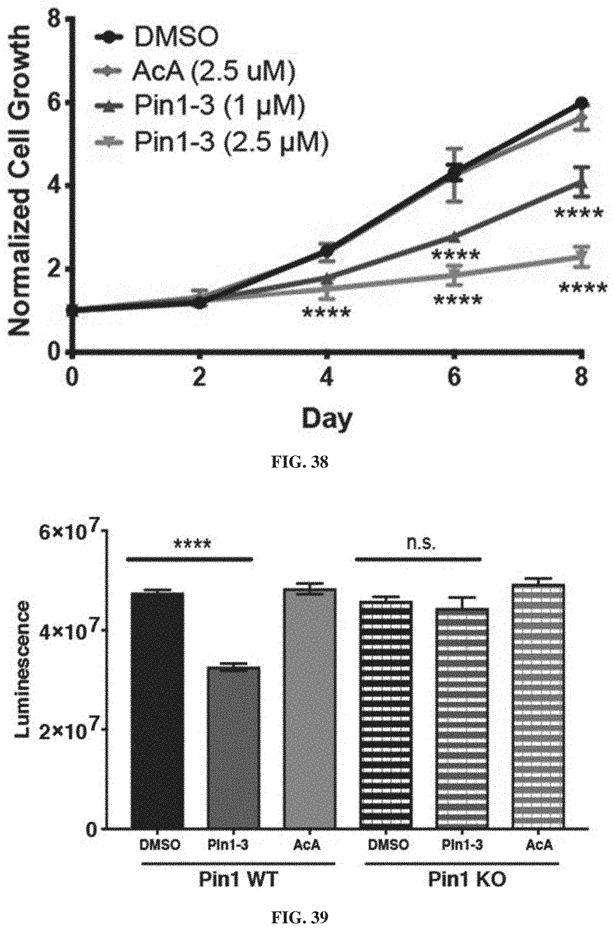

As shown in FIGS. 36-38, Pin1-3 exhibited long-term inhibition of PC3 prostate cancer cells (FIG. 36), Kuramochi ovarian carcinoma cells (FIG. 37) and MDA-MB-468 breast adenocarcinoma cells (FIG. 38), with the most pronounced effects being observed in MDA-MB-468 cells.

As three dimensional (3D) organoid models can reflect in vivo results better than monolayer cell culture [Baker et al., Trends Cancer Res 2016, 2:176-190], the anti-proliferative activity of Pin1-3 in PATU-8988T was further evaluated in wild-type or Pin1-knockout cells grown as organoids in Matrigel™ droplets. Cells were treated for 9 days with Pin1-3 (or Pin1-3-AcA or vehicle as a control), replenishing the compound in media every 3 days.

As shown in FIG. 39, Pin1-3 significantly retarded organoid growth in wild-type 8988T pancreatic cancer cells, but had no effect in Pin1-knockout pancreatic cancer cells, and the inactive Pin1-3-AcA control had no effect in either type of cell. The observed differences between wild-type and Pin1-knockout cells are indicative of an on-target phenotype.

The above results indicate that Pin1-binding compounds described herein can inhibit cancer cell growth in a wide variety of cancer cells, especially by affecting cell viability after prolonged treatment (e.g., as opposed to inducing proliferation defects at short time scales).

### Example 7

### Effect of Pin1-Binding Compound on Myc Transcription

In order to test whether Pin1-3 affects Myc transcriptional output, Mino B cells were treated with Pin1-3 (1 μM) for 6 hours (in triplicates) or vehicle (DMSO), followed by a global RNA sequencing analysis to detect differentially expressed genes as the result of this perturbation.

As shown in FIG. 40, 206 genes were found to be significantly down-regulated.

A gene set enrichment analysis of these genes was performed using Enrichr, as described in Kuleshov et al. [Nucleic Acids Res 2016, 44:W90-W97], against a dataset of genes identified by ChIP-seq (chromatin immunoprecipitation followed by sequencing) for various transcription factors.

As shown in FIG. 41, Myc target genes in K562 cells and HeLa-S3 cells appeared as the most enriched set and the 3rd most enriched set, respectively (adjusted p-value of 1.99×10⁻¹⁶ and 2.00×10⁻¹³ respectively) validating a significant downregulation of Myc's transcriptional signature by Pin1-3.

These results indicate that Pin1-binding compounds described herein can significantly downregulate Myc transcription.

### Example 8

### Effect of Pin1-Binding Compound in Neuroblastoma Model

The effect of Pin1-binding cells on neuroblastoma cells was assessed using a zebrafish embryo model of neuroblastoma, using procedures described in the Materials and Methods section hereinabove. Neuroblastoma is a pediatric malignancy derived from the peripheral sympathetic nervous system (PSNS). During the development of normal zebrafish embryos, neural crest-derived PSNS neuroblasts form the primordial superior cervical ganglia (SCG) and intrarenal gland (IRG) at the age of 3 to 7 days post fertilization (dpf), and can be visualized using the dβh:EGFP fluorescent reporter [He et al., Elife 2016, 5]. Overexpression of the MYCN oncogene, which is the oncogenic driver in approximately 20% of human high-risk neuroblastomas, in the PSNS of Tg(dβh:MYCN;dβh:EGFP) transgenic zebrafish, causes the fish to develop neuroblast hyperplasia (as shown, for example, in FIG. 42), which rapidly progress into fully transformed tumors that faithfully resemble human high-risk neuroblastoma [Zhu et al., Cancer Cell 2012, 21:362-373; He et al., Elife 2016, 5; Zimmerman et al., Cancer Discov 2016, 8:320-335].

As shown in FIGS. 42 and 43, Pin1-3 suppressed the hyperproliferation of MYCN-overexpressing PSNS neuroblasts over a 4 day period from 3 to 7 dpf, in a dose-dependent manner, at concentrations of 25 to 100 μM in the egg water. As further shown therein, after treatment with 100 μM concentration of the drug for 4 days, the cross-section of the EGFP-expressing PSNS cells is indistinguishable from that of controls without hyperproliferation.

In addition, no evidence of toxicity was observed in the embryos treated with Pin1-3 at the abovementioned concentrations, indicating further that Pin1-3 is well-tolerated by healthy tissues in vivo.

MYCN is one of very few genes that can initiate neuroblastoma when overexpressed in this zebrafish model. About 70-80% of MYCN-overexpressing fish with hyperproliferative PSNS neuroblasts at day 7 will go on to develop fully transformed neuroblastoma by 7 weeks of age.

The anti-tumor activity of Pin1-3 was then assessed on the maintenance of fully transformed neuroblastoma cells in vivo in primary tumor derived allograft (PDA) models constructed in transplanted zebrafish embryos. EGFP-labeled neuroblastoma cells were dissected from 4-month-old Tg(dβh:MYCN;dβh:EGFP) donor zebrafish, disaggregated, counted and 200-400 GFP-labeled tumor cells were injected intravenously into the Duct of Cuvier (common cardinal vein) of 2 dpf zebrafish embryos [He et al., J Pathol 2012, 227:431-445]. One day after injection, 100 μM Pin1-3 or the DMSO control was added to the fish water containing embryos bearing the transplanted EGFP-labeled neuroblastoma cells. Five days later, the area of the EGFP-labeled tumor mass in treated embryos was quantified.

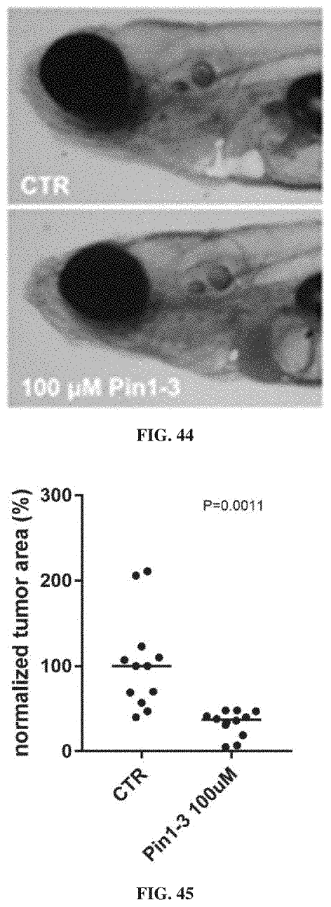

As shown in FIGS. 44 and 45, tumor masses in the DMSO-treated embryos grew larger over the five days of treatment, whereas the tumor masses decreased in size in the Pin1-3-treated embryos, indicating that Pin1-3 can not only suppress MYCN-driven neuroblastoma initiation, but also suppress the growth and survival in vivo of transplants of fully transformed primary neuroblastoma tumor cells.

The above results thus indicate that Pin1-binding compounds described herein can inhibit initiation of neuroblastomas (NB), particularly NB associated with MYCN expression.

### Example 9

### Pharmacokinetics and Pharmacodynamics of Exemplary Pin1-Binding Compound

The pharmacokinetics and pharmacodynamics of the exemplary compound Pin1-3 was assessed in a mouse model. Pin1-3 exhibited encouraging metabolic stability in mouse hepatic microsomes (T_{1/2}=41 minutes).

Male C57Bl/6J mice received Pin1-3 intravenously (as a 0.2 mg/ml solution in 5/5/90 NMP/Solutol/saline) or orally (as a 1 mg/ml solution in 5/5/90 NMP/Solutol/saline). The intravenous dosage was 2 mg/kg and the oral dosage was 10 mg/kg.

The results are summarized in Tables 5 and 6.

TABLE 5
Pharmacokinetic/pharmacodynamic parameters determined in 3 mice following
intravenous administration of 2 mg/kg Pin1-3 (obs. = observed, extrap. = extrapolated).

| | | | | | | | AUC_{INF} | | Cl | | |
|---|---|---|---|---|---|---|---|---|---|---|---|
| | | | | | AUCₗₐₛₜ | | obs. | AUC | obs. | MRT_{INF} | V_{SS} |
| | T_{1/2} | Tₘₐₓ | Cₘₐₓ | | min* | hr* | min* | % | ml/ | obs. | obs. |
| NO. | hr | hr | ng/ml | μM | ng/ml | μM | ng/ml | extrap. | min/kg | hr | L/kg |
| 1 | 0.72 | 0.50 | 2030 | 7.22 | 364891 | 21.6 | 365536 | 0.18 | 5.47 | 1.82 | 0.60 |
| 2 | 0.89 | 0.50 | 2610 | 9.28 | 431307 | 25.6 | 432853 | 0.36 | 4.62 | 1.70 | 0.47 |
| 3 | 0.68 | 0.50 | 1620 | 5.76 | 293517 | 17.4 | 294012 | 0.17 | 6.80 | 1.88 | 0.77 |
| Avg. | 0.76 | 0.50 | 2087 | 7.42 | 363238 | 21.5 | 364134 | 0.23 | 5.63 | 1.80 | 0.61 |

TABLE 6
Pharmacokinetic/pharmacodynamic parameters determined in 3 mice following oral
administration of 10 mg/kg Pin1-3 (obs. = observed, extrap. = extrapolated).

| | | | | | | | AUC_{INF} | | Cl | |
|---|---|---|---|---|---|---|---|---|---|---|
| | | | | | AUCₗₐₛₜ | | obs. | AUC | obs. | |
| | T_{1/2} | Tₘₐₓ | Cₘₐₓ | | min* | hr* | min* | % | ml/min/ | F % |
| No. | hr | hr | ng/ml | μM | ng/ml | μM | ng/ml | extrap. | kg | hr |
| 1 | 0.92 | 0.50 | 3200 | 11.38 | 585438 | 34.7 | 587646 | 0.38 | 17.02 | |
| 2 | 0.64 | 0.25 | 4050 | 14.41 | 575604 | 34.1 | 575764 | 0.03 | 17.37 | |
| 3 | 0.91 | 0.50 | 2420 | 8.61 | 496559 | 29.4 | 498172 | 0.32 | 20.07 | |
| Avg. | 0.82 | 0.42 | 3223 | 11.47 | 552534 | 32.8 | 553861 | 0.24 | 18.15 | 30.42 |

As shown in Table 6, oral administration of 10 mg/kg Pin1-3 resulted in an average Cₘₐₓ of 11.47 μM and oral bioavailability (F %) of 30.42, suggesting that Pin1-3 is suitable for oral in vivo dosing.

Toxicity of Pin1-3 was then evaluated in an acute toxic model. Mice were injected with 10, 20 or 40 mg/kg Pin1-3 intraperitoneally every day for two weeks. No adverse effects were recorded, weight was normal, and post-mortem examination found no pathologies.

These results indicate that Pin1-3 exhibits pharmacokinetics and nontoxicity suitable for in vivo use, including oral administration.

### Example 10

### Phenocopying of Pin1 Knockout Phenotypes

Phan et. al. [Nat Immunol 2007, 1132-1139] have reported that Pin1−/− mice display significantly larger germinal centers in response to immunization due to increased levels of BCL6. 12 wild-type mice were immunized with OVA coupled to the hapten 4-hydroxy-3-nitrophenylacetyl (NP-OVA) precipitated in alum. The mice were injected with two doses of Pin1-3 (IP; 40 mg/kg) or vehicle on days 7 and 9 post immunization, and on day 11 the mice were sacrificed and germinal centers size was assessed in lymph nodes by flow cytometry.

As shown in FIGS. 46A and 46B, Pin1-3 treated mice exhibited significantly larger germinal centers.

These results, in view of Phan et. al. [Nat Immunol 2007, 1132-1139], confirm the inhibition of Pin1 by Pin1-3.

### Example 11

### Effect of Exemplary Pin1-Binding Compound in Additional Cancer Models

Pancreatic ductal adenocarcinoma (PDAC) cells (derived from a human patient) were treated with Pin1-3 for 3 days. PDAC organoids were treated with Pin1-3 for 7 days (day 7 to day 14).

As shown in FIGS. 47 and 48, Pin1-3 inhibited tumor growth of PDAC cells in a dose-dependent manner.

As shown in FIG. 49, Pin1-3 reduced Pin1 in PDAC cells in a dose-dependent manner, indicating that Pin1 degradation was induced.

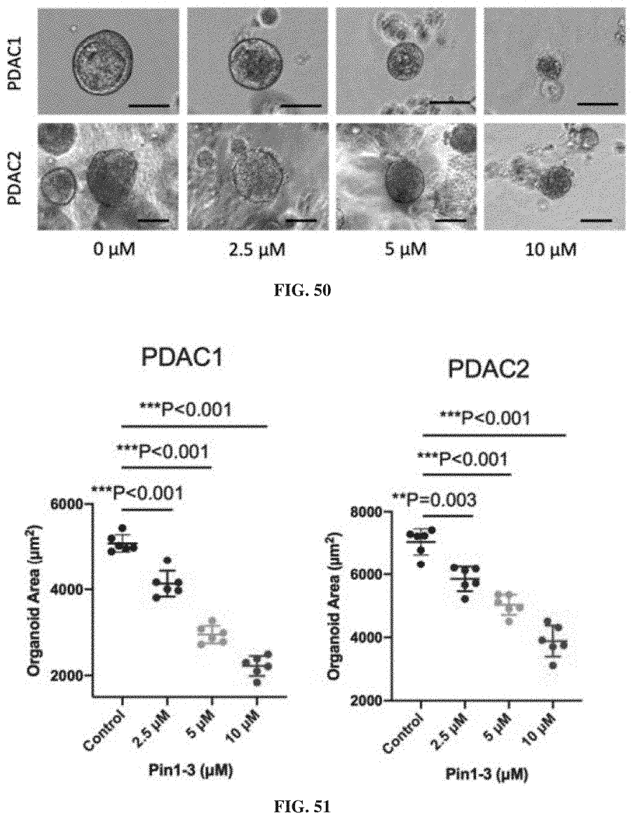

As shown in FIGS. 50 and 51, Pin1-3 inhibited PDAC organoid growth in a dose-dependent manner. 4×2 mm PDX (patient-derived xenograft) tumors were transplanted into NSC mouse pancreas (orthotopic xenografts). After 1 week of the transplantation, treatment of mice with Pin1-3 began. Mice were treated (IP) with Pin1-3 diluted solution (as a control), or 2 or 4 mg/kg Pin1-34 mg/kg every day. Tumor size were measured and mice were sacrificed after 6 weeks to collect tumor tissue (n=5).

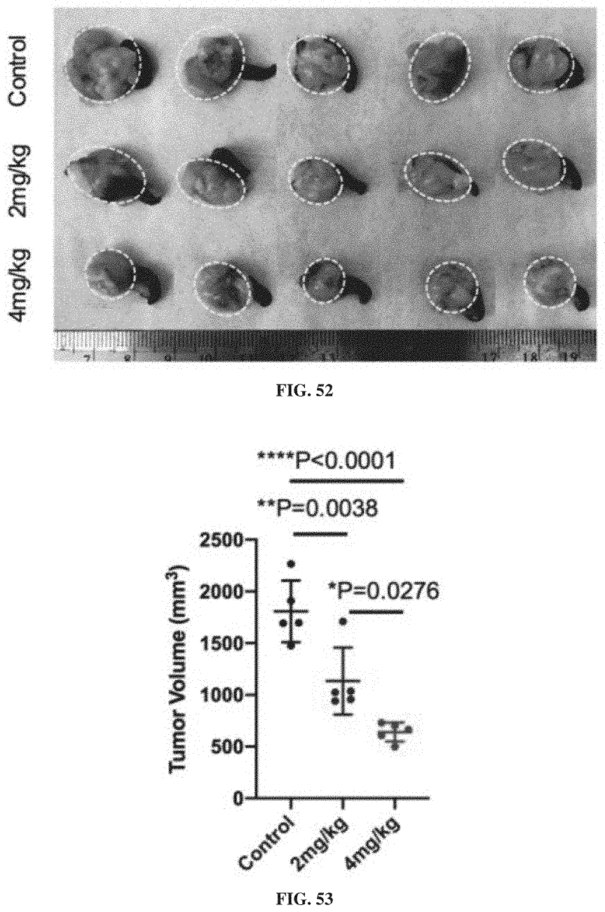

As shown in FIGS. 52-54, Pin1-3 inhibited PDX tumor growth in mice in a dose-dependent manner.

10⁶ KPC (KrasLSL.G12D/+; p53R₁₇₂H/+; PdxCretg/+) mouse derived tumor cells were transplanted into B6 mice pancreas (orthotopic transplantation). After 1 week of the transplantation, treatment of mice with Pin1-3 began. Mice were treated (IP) with Pin1-3 diluted solution (as a control), or 20 or 40 mg/kg every day. Tumor size was measured, and when the tumor size in control group reached 2 cm, mice were sacrificed to collect tumor tissue (n=4), and Kaplan-Meier survival analysis (n=8) was performed.

As shown in FIGS. 55-57, Pin1-3 inhibited KPC tumor growth and enhanced survival in mice.

These results further indicate that Pin1-binding compounds can effectively treat cancer.

### Example 12

### Chloroacetamide Preparation

General Procedure:

A general procedure for preparing sulfolane-containing chloroacetamides is depicted in Scheme 1:

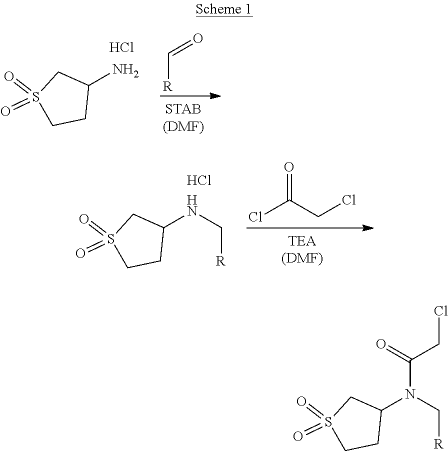

3-Aminosulfolane hydrochloride (1 eq.) is added to a solution of triethylamine (TEA) (0.9 eq.) in dry dimethylformamide (DMF) and stirred for 1 hour at room temperature. Afterwards, an aldehyde (1.1 eq.) and acetic acid (0.2 eq.) are added to the reaction mixture and stirred at room temperature for 1 hour. Sodium triacetoxyborohydride (STAB) (2.1 eq.) is then added at once to the mixture and stirred overnight at room temperature. After evaporation of the solvent, the residue is dissolved with saturated aqueous NaHCO₃, and the aqueous solution is extracted with ethyl acetate (2×). The organic layers are combined, dried over Na₂SO₄ and filtered. Evaporation of the solvent yields the secondary amine as hydrochloride, which is used without purification in the next step. Secondary amine hydrochloride (1 eq.) is dissolved in dry DMF and cooled to 0° C. Subsequently, 2-chloroacetyl chloride (1.2 eq.) and TEA (1.2 eq.) are added dropwise at 0° C. and stirred for 30 minutes. Afterwards, the reaction mixture is allowed to reach room temperature and stirred for 1 hour. The reaction is quenched at 0° C. by the addition of water.

Purification is effected by reverse phase high performance liquid chromatography (RP-HPLC)-linear gradient 5→95% ACN/H₂O+0.1% TFA in 30 minutes—and lyophilization yields the corresponding chloroacetamide.

### Preparation of 2-chloro-N-(sulfolan-3-yl)-N-neopentylacetamide (Pin1-3)

Using the above general procedure, the exemplary compound Pin1-3 (2-chloro-N-(sulfolan-3-yl)-N-neopentylacetamide) was prepared, as depicted in Scheme 2:

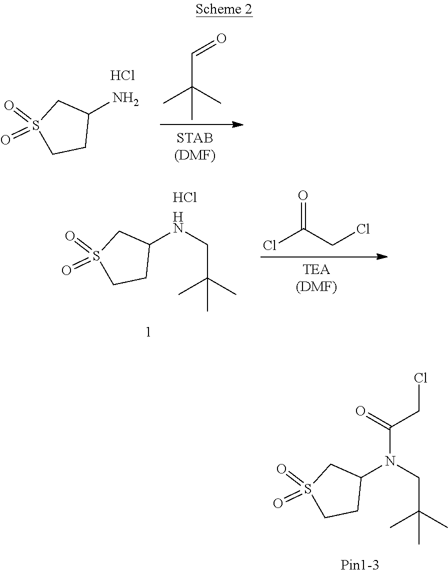

3-Aminosulfolane hydrochloride (100 mg, 0.583 mmol, 1 eq.) was added to a solution of triethylamine (TEA) (73.1 μl, 0.524 mmol, 0.9 eq.) in dry dimethylformamide (DMF) (1.4 ml) and stirred for 1 hour at room temperature. Afterwards, pivaldehyde (69.6 μl, 0.641 mmol, 1.1 eq.) and acetic acid (6.67 μl, 0.117 mmol, 0.2 eq.) were added to the reaction mixture and stirred at room temperature for 1 hour. Sodium triacetoxyborohydride (STAB) (259 mg, 1.223 mmol, 2.1 eq.) was then added at once to the mixture and stirred overnight at room temperature. After evaporation of the solvent, the residue was dissolved with saturated aqueous NaHCO₃ (0.5 ml) and the aqueous solution was extracted with ethyl acetate (2×1 ml). The organic layers were combined, dried over Na₂SO₄ and filtered. Evaporation of the solvent yielded the secondary amine Compound 1 as a white solid (86.2 mg, 0.42 mmol, 72% (crude product)), which was used without purification in the next step.

Compound 1 as hydrochloride (100 mg, 0.487 mmol, 1 eq.) was dissolved in dry DMF (1 ml) and cooled to 0° C. Subsequently, 2-chloroacetyl chloride (46.8 μl, 0.584 mmol, 1.2 eq.) and TEA (81 μl, 0.584 mmol, 1.2 eq.) were added dropwise at 0° C. and stirred for 30 minutes. Afterwards, the reaction mixture was allowed to reach room temperature and stirred for 2 hours. The reaction was quenched at 0° C. by the addition of water (2 ml).

Purification of Pin1-3 was effected by reverse phase high performance liquid chromatography (RP-HPLC)−t_{R}=16 minutes, linear gradient 5→95% ACN/H₂O+0.1% TFA in 30 minutes—and lyophilization yielded chloroacetamide Pin1-3 (59.83 mg, 0.212 mmol, 43.6% (last step)) as white powder.

¹H-NMR (500 MHz, CDCl₃): δ=4.11 (d, J=5.50 Hz, 2H), 3.89-4.00 (m, 1H), 3.66-3.78 (m, 2H), 3.25-3.34 (m, 1H), 3.10-3.20 (m, 2H), 3.00-3.09 (m, 1H), 2.47-2.61 (m, 2H), 1.03 (s, 9H) ppm.

¹³C (126 MHz, CDCl₃): δ=168.0, 62.4, 57.6, 50.3, 49.0, 42.1, 33.6, 28.0, 26.6 ppm.

MS (ESI): m/z calcd. for C₁₁H₂₁ClNO₃S⁺[M+H⁺]: 282.10; found 282.29.

### Preparation of 2-chloro-N-(sulfolan-3-yl)-N-isobutylacetamide (Pin1-3-15)

Using the above general procedure, the exemplary compound Pin1-3-15 (2-chloro-N-(sulfolan-3-yl)-N-isobutylacetamide) was prepared.

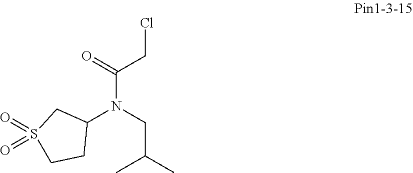 [SMILES: CC(C)CN(C(=O)CCl)C1CCS(=O)(=O)C1]

3-Aminosulfolane hydrochloride (90 mg, 0.524 mmol, 1 eq.) was added to a solution of triethylamine (TEA) (65.8 μl, 0.474 mmol, 0.9 eq.) in dry dimethylformamide (DMF) (1.3 ml) and stirred for 1 hour at room temperature. Afterwards, isobutyraldehyde (57.4 μl, 0.629 mmol, 1.2 eq.), acetic acid (6 μl, 0.105 mmol, 0.2 eq.) and sodium triacetoxyborohydride (STAB) (233 mg, 1.101 mmol, 2.1 eq.) were added to the reaction mixture and stirred overnight at room temperature. After workup and evaporation of the solvent, the secondary amine (78.18 mg, 0.343 mmol, 65.5% (crude product)) was used without purification in the next step.

2-Chloroacetyl chloride (33 μl, 0.412 mmol, 1.2 eq.) and triethylamine (57.4 μl, 0.412 mmol, 1.2 eq.) were added dropwise to cooled (0° C.) secondary amine hydrochloride (78.18 mg, 0.487 mmol, 1 eq.) in dry dimethylformamide (1 ml) and stirred for 30 minutes, and then quenched with water (2 ml).

Purification of Pin1-3-15 was effected by reverse phase high performance liquid chromatography (RP-HPLC)−t_{R}=14 minutes, linear gradient 5→95% ACN/H₂O+0.1% TFA in 30 minutes—yielding Pin1-3-15 (29.22 mg, 0.412 mmol, 31.8%) as white powder.

¹H-NMR (500 MHz, CDCl₃): δ=4.02-4.17 (m, 3H), 3.67-3.76 (m, 1H), 3.62 (dt, J=12.10, 8.80 Hz, 1H), 3.03-3.24 (m, 5H), 2.44-2.58 (m, 2H), 1.93 (dt, J=13.20, 6.60 Hz, 1H), 0.99 (t, J=6.60 Hz, 6H) ppm.

¹³C (126 MHz, CDCl₃): δ=167.0, 58.0, 55.2, 50.5, 49.7, 42.0, 28.4, 26.2, 19.9, 19.7 ppm.

MS (ESI): m/z calcd. for C₁₀H₁₉ClNO₃S⁺[M+H⁺]: 268.08; found 268.29.

### Preparation of 2-chloro-N-(sulfolan-3-yl)-N-(cyclopentylmethyl)acetamide (Pin1-3-14)

Using the above general procedure, the exemplary compound Pin1-3-14 (2-chloro-N-(sulfolan-3-yl)-N-(cyclopentylmethyl)acetamide) was prepared.

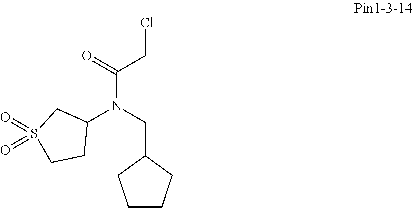 [SMILES: O=C(CCl)N(CC1CCCC1)C1CCS(=O)(=O)C1]

3-Aminosulfolane hydrochloride (100 mg, 0.583 mmol, 1 eq.) and triethylamine (73.1 μl, 0.524 mmol, 0.9 eq.) in dry dimethylformamide (DMF) (1.3 ml) were stirred for 1 hour at room temperature. Afterwards, cyclopentanecarboxaldehyde (68.4 μl, 0.641 mmol, 1.1 eq.), acetic acid (6.67 μl, 0.117 mmol, 0.2 eq.) and sodium triacetoxyborohydride (STAB) (259 mg, 1.223 mmol, 2.1 eq.) were added to the reaction mixture and stirred overnight at room temperature. After workup and evaporation, the secondary amine (95.68 mg, 0.377 mmol, 64.7% (crude product)) was used without purification in the next step.

2-Chloroacetyl chloride (36.2 μl, 0.452 mmol, 1.2 eq.) and triethylamine (63.1 μl, 0.452 mmol, 1.2 eq.) were added dropwise to cooled (0° C.) secondary amine hydrochloride (95.68 mg, 0.377 mmol, 1 eq.) in dry DMF (1 ml) and stirred for 30 minutes, and then quenched with water (2 ml).

Purification of Pin1-3-14 was effected by reverse phase high performance liquid chromatography (RP-HPLC)−t_{R}=17.5 minutes, linear gradient 5→95% ACN/H₂O+0.1% TFA in 30 minutes—yielding Pin1-3-14 (23.4 mg, 0.08 mmol, 21.13% (last step)) as white powder.

¹H-NMR (500 MHz, CDCl₃): δ=4.11 (m, 3H), 3.57-3.76 (m, 2H), 3.22-3.41 (m, 2H), 3.15 (dd, J=12.10, 8.80 Hz, 1H), 3.03-3.10 (m, 1H), 2.46-2.58 (m, 2H), 2.10-2.21 (m, 1H), 1.78-1.94 (m, 2H), 1.60-1.78 (m, 4H), 1.17-1.29 (m, 2H) ppm.

¹³C (126 MHz, CDCl₃): δ=166.8, 55.3, 55.1, 50.5, 49.7, 42.0, 40.2, 30.4, 30.4, 26.3, 24.9, 24.9 ppm.

MS (ESI): m/z calcd. for C₁₂H₂₁ClNO₃S⁺ [M+H⁺]: 294.10; found 294.31.

### Preparation of 2-chloro-N-(sulfolan-3-yl)-N-(cyclohexylmethyl)acetamide (Pin1-2-3)

Using the above general procedure, the exemplary compound Pin1-2-3 (2-chloro-N-(sulfolan-3-yl)-N-(cyclohexylmethyl)acetamide) was prepared.

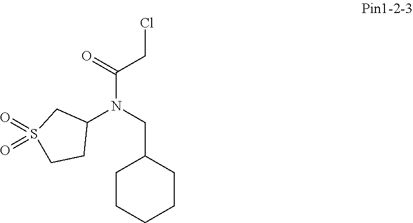 [SMILES: O=C(CCl)N(CC1CCCCC1)C1CCS(=O)(=O)C1]

3-Aminosulfolane hydrochloride (75 mg, 0.437 mmol, 1 eq.) in dry dimethylformamide (DMF) (1.3 ml) were stirred for 1 hour at room temperature. Afterwards, cyclohexanecarboxaldehyde (58.2 μl, 0.481 mmol, 1.1 eq.) and sodium triacetoxyborohydride (STAB) (139 mg, 0.655 mmol, 1.5 eq.) were added to the reaction mixture and stirred overnight at room temperature. After workup and evaporation, the secondary amine (72.11 mg, 0.269 mmol, 62% (crude product)) was used without purification in the next step.

2-Chloroacetyl chloride (24.8 μl, 0.323 mmol, 1.2 eq.) and triethylamine (45 μl, 0.323 mmol, 1.2 eq.) were added dropwise to cooled (0° C.) secondary amine hydrochloride (72 mg, 0.269 mmol, 1 eq.) in dry DMF (0.5 ml) and stirred for 30 minutes, and then quenched with water (2 ml).

Purification of Pin1-2-3 was effected by reverse phase high performance liquid chromatography (RP-HPLC)−t_{R}=18.5 minutes, linear gradient 5→95% ACN/H₂O+0.1% TFA in 30 minutes—yielding Pin1-2-3 (9.1 mg, 0.030 mmol, 11% (last step)) as white powder.

¹H-NMR (500 MHz, CDCl₃): δ=4.01-4.15 (m, 2H), 3.68-3.75 (m, 1H), 3.62 (dt, J=13.20, 8.80 Hz, 1H), 3.04-3.25 (m, 4H), 2.43-2.58 (m, 2H), 1.66-1.85 (m, 5H), 1.57 (m, 1H), 1.14-1.33 (m, 3H), 0.90-1.03 (m, 2H) ppm.

¹³C (126 MHz, CDCl₃): δ=167.0, 57.1, 55.3, 50.5, 49.7, 42.0, 38.0, 30.9, 30.8, 26.2, 26.1, 25.8 ppm.

MS (ESI): m/z calcd. for C₁₃H₂₃ClNO₃S⁺ [M+H⁺]: 308.11; found 308.28.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

In addition, any priority document(s) of this application is/are hereby incorporated herein by reference in its/their entirety.

What is claimed is:

1. A method of modulating an activity of Pin1, the method comprising contacting the Pin1 with a compound comprising an electrophilic moiety and rigid moiety that comprises at least one functional group that is capable of forming hydrogen bonds with hydrogen atoms, wherein said electrophilic moiety comprises a haloalkyl, and wherein said electrophilic moiety and said rigid moiety are arranged such that said electrophilic moiety is capable of covalently binding to the Cys113 residue of said Pin1, and said rigid moiety is capable of forming hydrogen bonds with the Gln131 and His 157 residues of said Pin1.

2. The method of claim 1, wherein said electrophilic moiety comprises a haloacetamide.

3. The method of claim 1, wherein said functional group is capable of forming a hydrogen bond with a backbone amide hydrogen of said Gln131 and/or with an imidazole NH of said His157.

4. The method of claim 1, wherein said functional group is an oxygen atom.

5. The method of claim 1, wherein said rigid moiety comprises a sulfone group.

6. The method of claim 1, wherein said compound further comprises a hydrophobic moiety.

7. The method of claim 1, wherein said compound has a molecular weight lower than 500 Da.

8. The method of claim 1, wherein said compound is represented by Formula I:
E-L₁-G(F)m Formula I
wherein:
E is said electrophilic moiety;
L₁ is a bond or a linking moiety;
G is said rigid moiety;
F are each said functional moiety forming said hydrogen bonds; and
m is 2, 3 or 4.

9. The method of claim 8, wherein said compound is represented by Formula Ia: 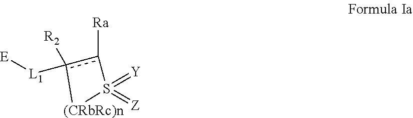
wherein:
the dashed line represents a saturated or non-saturated bond;
Y and Z are each independently selected from the group consisting of O, S and NH;
R₂ and Ra-Rc are each independently selected from the group consisting of hydrogen, alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino, or alternatively, R₂ is absent when the dashed line represents an unsaturated bond; and
n is 1, 2, 3 or 4.

10. The method of claim 9, wherein said compound is represented by Formula Ib: 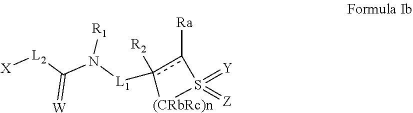
wherein:
W is selected from the group consisting of O, S and NR₃;
X is halo;
Ra-Rc are each hydrogen;
L₁ is a bond or alkylene;
L₂ is alkylene; and
R₁ and R₃ are each independently selected from the group consisting of hydrogen, alkyl, alkenyl, alkynyl, cycloalkyl, heteroalicyclic, aryl and heteroaryl.

11. The method of claim 8, wherein said compound is represented by Formula Ic: 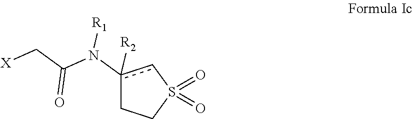
wherein:
the dashed line represents a saturated or non-saturated bond;
X is halo;
R₁ is selected from the group consisting of alkyl, alkenyl, alkynyl, cycloalkyl, heteroalicyclic, aryl and heteroaryl; and
R₂ is selected from the group consisting of hydrogen and alkyl when the dashed line represents a saturated bond, and R₂ is absent when the dashed line represents an unsaturated bond.

12. The method of claim 10, wherein R₁ has Formula II:
—CH₂—R′₁ Formula II
wherein R′₁ is selected from the group consisting of alkyl, alkenyl, alkynyl, cycloalkyl, aryl, heteroaryl, heteroalicyclic, halo, hydroxy, alkoxy, aryloxy, thiohydroxy, thioalkoxy, thioaryloxy, sulfinyl, sulfonyl, sulfonate, sulfate, cyano, nitro, azide, phosphonyl, phosphinyl, carbonyl, thiocarbonyl, a urea group, a thiourea group, O-carbamyl, N-carbamyl, O-thiocarbamyl, N-thiocarbamyl, C-amido, N-amido, C-carboxy, O-carboxy, sulfonamido, guanyl, guanidinyl, hydrazine, hydrazide, thiohydrazide, and amino.

13. The method of claim 12, wherein R′₁ is a tertiary alkyl, alkenyl, alkynyl, cycloalkyl or heteroalicyclic.

14. The method of claim 13, wherein R′₁ is a substituted or unsubstituted t-butyl.

15. The method of claim 1, being for treating a condition in which modulating an activity of Pin1 is beneficial, the method comprising administering said compound to a subject in need thereof.

16. The method according to claim 15, wherein said condition is a proliferative disease or disorder and/or an immune disease or disorder.

17. A compound having Formula Id: 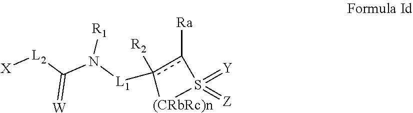
wherein:
the dashed line represents a saturated or non-saturated bond;
W is selected from the group consisting of O, S and NR₃;
X is halo;
Y and Z are each independently selected from the group consisting of O, S and NH;
Ra-Rc are each hydrogen;
L₁ is a bond or alkylene;
L₂ is alkylene;
n is 1, 2, 3 or 4;
R₁ is selected from the group consisting of —CH₂—C(CH₃)₃, a triazole, and alkyl substituted by a triazole and/or by a 5- or 6-membered cycloalkyl;
R₂ is selected from the group consisting of hydrogen and alkyl when the dashed line represents a saturated bond, and R₂ is absent when the dashed line represents an unsaturated bond; and
R₃ is selected from the group consisting of hydrogen, alkyl, alkenyl, alkynyl, cycloalkyl, heteroalicyclic, aryl and heteroaryl,
wherein said triazole has Formula III: 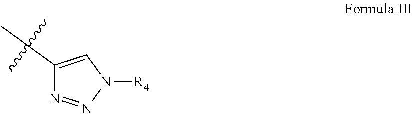
wherein R₄ is selected from the group consisting of alkyl, alkenyl, alkynyl, cycloalkyl, heteroalicyclic, aryl and heteroaryl.

18. The compound of claim 17, wherein n is 2.

19. The compound of claim 17, wherein Y and Z are each O.

20. The compound of claim 17, wherein L₁ is a bond.

21. The compound of claim 17, wherein the dashed line represents a saturated bond.

22. The compound of claim 17, wherein X is chloro.

23. The compound of claim 17 wherein R₄ is a substituted or unsubstituted phenyl.

24. A screening library comprising at least 30 compounds according to claim 17.

25. A method of modulating an activity of Pin1, the method comprising contacting the Pin1 with the compound of claim 17.

26. A method of identifying a compound capable of modulating an activity of Pin1, the method comprising screening a library comprising at least 30 compounds having Formula IV:
E′-L′₁-V Formula IV
wherein:
E′ is an electrophilic moiety as defined in claim 1, capable of forming a covalent bond when reacted with a thiol;
L′₁ is a linking moiety;
V is a moiety featuring at least two functional groups that are capable of forming hydrogen bonds, and optionally further features at least one lipophilic group,
for compounds that are capable of interacting with a Cys113 residue of said Pin1 via said electrophilic moiety, of interacting at least with the Gln131 and His 157 residues of said Pin1 via said functional groups, and optionally of interacting with at least one amino acid residue in a hydrophobic patch of said Pin1 via said at least one lipophilic group,
wherein a compound identified as capable of said interacting at least with said Cys113 residue and said Gln131 and His 157 residues of said Pin1 is identified as capable of modifying an activity of said Pin1.

27. A screening library comprising at least 30 compounds represented by Formula Ic: 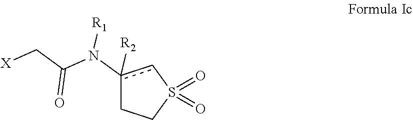
wherein:
the dashed line represents a saturated or non-saturated bond;
X is halo;
R₁ is selected from the group consisting of alkyl, alkenyl, alkynyl, cycloalkyl, heteroalicyclic, aryl and heteroaryl; and
R₂ is selected from the group consisting of hydrogen and alkyl when the dashed line represents a saturated bond, and R₂ is absent when the dashed line represents an unsaturated bond.

28. A method of identifying a compound capable of modulating an activity of Pin1, the method comprising:
a) contacting the library of claim 27 with Pin1 under conditions that allow nucleophilic substitution of said X by a Cys113 residue of Pin1; and
b) determining which compounds covalently bound Pin1, wherein a compound which covalently binds to Pin1 is identified as being capable of modulating an activity of Pin1.

29. The method of claim 28, further comprising screening said library for low reactivity with a thiol other than Cys113 of Pin1.


SEQUENCE LISTING (2 sequences)

SEQ ID NO: 1 - protein, 163 aa, Artificial Sequence
  amino acid residues of Pin1
MADEEKLPPGWEKRMSRSSGRVYYFNHITNASQWERPSGNSSSGGKNGQGEPARVRCSHLLVKHSQSRRPSSWRQEKITRTKEEALELINGYIQKIKSGEEDFESLASQFSDCSSAKARGDLGAFSRGQMQKPFEDASFALRTGEMSGPVFTDSGIHIILRTE

SEQ ID NO: 2 - protein, 4 aa, Artificial Sequence
  synthetic peptide
  MISC_FEATURE (1)..(1): N-Succinyl conjugated
  MISC_FEATURE (2)..(2): Phosphorylated
  MISC_FEATURE (4)..(4): 3′ p-nitroanilide conjugated
ASPF